SEQUENCE LISTING (32 sequences)

SEQ ID NO: 1 - protein, 51 aa, Artificial Sequence
  Description of Artificial Sequence Synthetic polypeptide
  MISC_FEATURE (1)..(20): This region may encompass 0-20 residues
  MISC_FEATURE (32)..(51): This region may encompass 0-20 residues
  See specification as filed for detailed description of substitutions and preferred embodiments
GGGGGGGGGGGGGGGGGGGGRGRDDRRQRRRGGGGGGGGGGGGGGGGGGGG

SEQ ID NO: 2 - protein, 51 aa, Artificial Sequence
  Description of Artificial Sequence Synthetic polypeptide
  MISC_FEATURE (1)..(20): This region may encompass 0-20 residues
  MISC_FEATURE (32)..(51): This region may encompass 0-20 residues
  See specification as filed for detailed description of substitutions and preferred embodiments
GGGGGGGGGGGGGGGGGGGGYGRKKRRQRRRGGGGGGGGGGGGGGGGGGGG

SEQ ID NO: 3 - protein, 49 aa, Artificial Sequence
  Description of Artificial Sequence Synthetic polypeptide
  MISC_FEATURE (1)..(20): This region may encompass 0-20 residues
  MISC_FEATURE (30)..(49): This region may encompass 0-20 residues
  See specification as filed for detailed description of substitutions and preferred embodiments
GGGGGGGGGGGGGGGGGGGGRKKRRQRRRGGGGGGGGGGGGGGGGGGGG

SEQ ID NO: 4 - protein, 35 aa, Artificial Sequence
  Description of Artificial Sequence Synthetic polypeptide
RKKRRQRRRGKKKKKKKKKKKKKKKGRKKRRQRRR

SEQ ID NO: 5 - protein, 45 aa, Artificial Sequence
  Description of Artificial Sequence Synthetic polypeptide
  MISC_FEATURE (1)..(20): This region may encompass 0-20 residues
  MISC_FEATURE (21)..(45): This region may encompass 5, 7, 9, 11, 13, 15, 17, 19, 21, 23, or 25 residues
  See specification as filed for detailed description of substitutions and preferred embodiments
GGGGGGGGGGGGGGGGGGGGRRRRRRRRRRRRRRRRRRRRRRRRR

SEQ ID NO: 6 - protein, 19 aa, Drosophila sp.
SGRQIKIWFQNRRMKWKKC

SEQ ID NO: 7 - protein, 39 aa, Artificial Sequence
  Description of Artificial Sequence Synthetic polypeptide
YGRKKRRQRRRGKKKKKKKKKKKKKKKGYGRKKRRQRRR

SEQ ID NO: 8 - protein, 39 aa, Artificial Sequence
  Description of Artificial Sequence Synthetic polypeptide
RGRDDRRQRRRGKKKKKKKKKKKKKKKGRGRDDRRQRRR

SEQ ID NO: 9 - protein, 20 aa, Artificial Sequence
  Description of Artificial Sequence Synthetic peptide
  MISC_FEATURE (1)..(20): This sequence may encompass 10-20 residues
  See specification as filed for detailed description of substitutions and preferred embodiments
KKKKKKKKKKKKKKKKKKKK

SEQ ID NO: 10 - protein, 10 aa, Artificial Sequence
  Description of Artificial Sequence Synthetic peptide
GGGRRRRRRR

SEQ ID NO: 11 - protein, 27 aa, Artificial Sequence
  Description of Artificial Sequence Synthetic peptide
  N-term palmitoyl
  MISC_FEATURE (1)..(20): This region may encompass 0-20 residues
  See specification as filed for detailed description of substitutions and preferred embodiments
GGGGGGGGGGGGGGGGGGGGKKRPKPG

SEQ ID NO: 12 - protein, 27 aa, Artificial Sequence
  Description of Artificial Sequence Synthetic peptide
  N-term octanoyl
  MISC_FEATURE (1)..(20): This region may encompass 0-20 residues
  See specification as filed for detailed description of substitutions and preferred embodiments
GGGGGGGGGGGGGGGGGGGGKKRPKPG

SEQ ID NO: 13 - protein, 27 aa, Artificial Sequence
  Description of Artificial Sequence Synthetic peptide
  N-term oleyl
  MISC_FEATURE (1)..(20): This region may encompass 0-20 residues
  See specification as filed for detailed description of substitutions and preferred embodiments
GGGGGGGGGGGGGGGGGGGGKKRPKPG

SEQ ID NO: 14 - protein, 24 aa, Artificial Sequence
  Description of Artificial Sequence Synthetic peptide
  MISC_FEATURE (8)..(17): This region may encompass 0-10 residues
  See specification as filed for detailed description of substitutions and preferred embodiments
FFFILVFGGGGGGGGGGKKRPKPG

SEQ ID NO: 15 - protein, 23 aa, Artificial Sequence
  Description of Artificial Sequence Synthetic peptide
  MISC_FEATURE (7)..(16): This region may encompass 0-10 residues
  See specification as filed for detailed description of substitutions and preferred embodiments
FLVFFFGGGGGGGGGGKKRPKPG

SEQ ID NO: 16 - protein, 23 aa, Artificial Sequence
  Description of Artificial Sequence Synthetic peptide
  MISC_FEATURE (8)..(17): This region may encompass 0-10 residues
  See specification as filed for detailed description of substitutions and preferred embodiments
KKRPKPGGGGGGGGGGGFLVFFF

SEQ ID NO: 17 - protein, 7 aa, Unknown
  Description of Unknown Efficiency group sequence
KKRPKPG

SEQ ID NO: 18 - protein, 12 aa, Unknown
  Description of Unknown Prion sequence
AAVLLPVLLAAP

SEQ ID NO: 19 - protein, 9 aa, Artificial Sequence
  Description of Artificial Sequence Synthetic peptide
RRRRRRRRR

SEQ ID NO: 20 - protein, 15 aa, Drosophila sp.
RQIKWFQNRRMKWKK

SEQ ID NO: 21 - protein, 38 aa, Unknown
  Description of Unknown Transduction Domain 1 sequence
NPGGYCLTKWMILAAELKCFGNTAVAKCNVNHDAEFCD

SEQ ID NO: 22 - protein, 26 aa, Unknown
  Description of Unknown Melittin sequence
GIGAVLKVLTTGLPALISWIKRKRQQ

SEQ ID NO: 23 - protein, 47 aa, Artificial Sequence
  Description of Artificial Sequence Synthetic polypeptide
  MISC_FEATURE (1)..(20): This region may encompass 0-20 residues
  MISC_FEATURE (28)..(47): This region may encompass 0-20 residues
  See specification as filed for detailed description of substitutions and preferred embodiments
GGGGGGGGGGGGGGGGGGGGKKRPKPGGGGGGGGGGGGGGGGGGGGG

SEQ ID NO: 24 - protein, 8 aa, Unknown
  Description of Unknown Efficiency group sequence
FLVFFFGG

SEQ ID NO: 25 - protein, 47 aa, Artificial Sequence
  Description of Artificial Sequence Synthetic polypeptide
  MISC_FEATURE (1)..(20): This region may encompass 0-20 residues
  MISC_FEATURE (28)..(47): This region may encompass 0-20 residues
  See specification as filed for detailed description of substitutions and preferred embodiments
GGGGGGGGGGGGGGGGGGGGKKRPQPDGGGGGGGGGGGGGGGGGGGG

SEQ ID NO: 26 - protein, 17 aa, Artificial Sequence
  Description of Artificial Sequence Synthetic peptide
KKRPKPGGGGFFFILVF

SEQ ID NO: 27 - protein, 17 aa, Artificial Sequence
  Description of Artificial Sequence Synthetic peptide
FFFILVFGGGKKRPKPG

SEQ ID NO: 28 - protein, 11 aa, Artificial Sequence
  Description of Artificial Sequence Synthetic peptide
GGGGKKRPKPG

SEQ ID NO: 29 - protein, 20 aa, Artificial Sequence
  Description of Artificial Sequence Synthetic peptide
RKKRRQRRRGGGGFFFILVF

SEQ ID NO: 30 - protein, 13 aa, Artificial Sequence
  Description of Artificial Sequence Synthetic peptide
GGGGRKKRRQRRR

SEQ ID NO: 31 - protein, 11 aa, Artificial Sequence
  Description of Artificial Sequence Synthetic peptide
  N-term palmitoyl
GGRKKRRQRRR

SEQ ID NO: 32 - protein, 7 aa, Artificial Sequence
  Description of Artificial Sequence Synthetic peptide
KLAKLAK

(19) United States
(12) Patent Application Publication
(10) Pub. No.: US 2020/0390871 A1
(43) Pub. Date: Dec. 17, 2020

(54) BOTULINUM TOXIN FORMULATIONS AND METHODS OF USE THEREOF IN PLANTAR FASCITIS WITH EXTENDED DURATION OF EFFECT

(71) Applicant: REVANCE THERAPEUTICS, INC., Newark, CA (US)

(72) Inventor: Roman Rubio, Redwood City, CA (US)

(21) Appl. No.: 16/761,432

(22) PCT Filed: Nov. 5, 2018
(86) PCT No.: PCT/US2018/059265

§ 371 Date: May 4, 2020

Related U.S. Application Data
(60) Provisional application No. 62/581,390, filed on Nov. 3, 2017.
(60) Provisional application No. 62/611,540, filed on Dec. 28, 2017.

(51) Int. Cl. A61K 38/48; A61K 47/64; A61P 19/04; A61K 9/00

(52) U.S. Cl. CPC A61K 38/4893; A61K 9/0019; A61P 19/04; A61K 47/6455

(57) ABSTRACT

This invention relates to injectable and transdermal compositions comprising botulinum toxin and their methods of use in administering botulinum toxin to treat or manage plantar fasciitis, a disorder related thereto, or a symptom thereof. The injectable and transdermal compositions and methods in which these compositions are used provide advantageous treatments which result in fast onset, higher responder rates, and/or long duration of effect, for example, a duration of effect for over six months and/or a reduction in plantar fasciitis pain by at least 50% maintained through week 8 following treatment. The topical compositions and methods provide desirable, less painful, treatment alternatives.

### CROSS REFERENCE TO RELATED CASE

This application claims benefit of priority to U.S. Provisional Patent Application No. 62/581,390, entitled “Botulinum Toxin Formulations And Methods Of Use Thereof In Plantar Fasciitis,” to Rubio, filed on Nov. 3, 2017, and claims benefit of priority to U.S. Provisional Patent Application No. 62/611,540, entitled “Botulinum Toxin Formulations And Methods Of Use Thereof In Plantar Fasciitis With Extended Duration Of Effect” to Rubio, filed Dec. 28, 2017. Both U.S. Provisional Patent Application Nos. 62/581,390 and 62/611,540 are incorporated herein in their entireties.

### FIELD OF THE INVENTION

This invention relates to injectable and transdermal compositions comprising botulinum toxin and their methods of use in administering botulinum toxin to treat or manage plantar fasciitis, a disorder related thereto, or a symptom thereof. The injectable compositions and methods in which these compositions are used provide advantageous treatments which result in fast onset, high responder rates, and/or long duration of effect, for example, a duration of effect for over 20 to 40 weeks and longer and/or a reduction in plantar fasciitis pain by at least 50% maintained through week 8 following treatment. The topical compositions and methods provide desirable, less painful, treatment alternatives.

### BACKGROUND OF THE INVENTION

Plantar fasciitis is a painful affliction, caused by inflammation of the ligament running along the bottom of the foot. It is the most common cause of heel pain and the most common foot condition treated by health care providers. An estimated one in ten of the general population will develop plantar fasciitis during their lifetime, and as much as 10-18 million individuals in the U.S. are affected by plantar fasciitis each year. Symptoms include sharp, constant pain that can last six to nine months or more. The pain can get worse over time, becoming debilitating, even requiring surgery.

Plantar fasciitis is caused by inflammation of the plantar fascia which is connective tissue in the arch of the foot. While its exact etiology is multifactorial and remains incompletely elucidated, it is understood that the plantar fascia is the foot's shock absorber so that repeated pressure on the tissue, from repetitive sports activity, repetitive trauma, aging, obesity, abnormal foot posture, or use of poor footwear, creates small tears and overstretches the fascia, increasing the risk of plantar fasciitis.

Present treatment options depend on the severity of the affliction. Treatments for less severe cases of plantar fasciitis include leg and foot stretching exercises, to stretch the plantar fascia and gastrocnemius/soleus complex muscles, as well as manual therapy, nonsteroidal anti-inflammatory drugs, corrective shoe inserts, heel pads, taping, splinting, and/or night splints. More severe or refractory cases are treated with steroid injections (such as cortical injections), platelet rich plasma injections, honophoresis, ultrasound, electrotherapy, extracorporeal shock wave therapy, surgery, and/or traditional botulinum toxin injections.

The type A form of botulinum toxin is reported to be the most lethal natural biological agent known to man. Spores of Clostridium botulinum are found in soil and can grow in improperly sterilized and sealed food containers. Botulism, which may be fatal, can be caused by the ingestion of the bacteria that produce the toxin. Botulinum toxin acts to produce paralysis of muscles, preventing synaptic transmission by inhibiting the release of acetylcholine across the neuromuscular junction, and is thought to act in other ways as well. Its action essentially blocks signals that normally would cause muscle spasms or contractions, resulting in paralysis. During the last decade, botulinum toxin's muscle paralyzing activity has been harnessed to achieve a variety of therapeutic effects. Controlled administration of botulinum toxin has been used to provide muscle paralysis to treat a variety of medical conditions, in particular, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temporomandibular joint disorder, and various types of muscle cramping and spasms. More recently, the muscle-paralyzing effects of botulinum toxin have been applied to therapeutic and cosmetic facial applications such as treatment of wrinkles, frown lines, and other results of spasms or contractions of facial muscles.

In addition to the type A form of botulinum toxin, there are seven other serologically distinct forms of botulinum toxin that are also produced by the gram-positive bacteria C. botulinum. Of these eight serologically distinct types of botulinum toxin, the seven that can cause paralysis have been designated botulinum toxin serotypes A, B, C, D, E, F and G. Each of these is distinguished by neutralization with type-specific antibodies. The different serotypes of botulinum toxin vary in the effect and in the severity and duration of the paralysis they evoke in different animal species. For example, in rats, it has been determined that botulinum toxin type A is 500 times more potent than botulinum toxin type B, as measured by the rate of paralysis. Additionally, botulinum toxin type B has been determined to be non-toxic in primates at a dose of 480 U/kg, about 12 times the primate LD₅₀ for type A.

As released by C. botulinum bacteria, botulinum toxin is a component of a toxin complex containing the approximately 150 kD botulinum toxin protein molecule along with associated non-toxin proteins. These endogenous non-toxin proteins are believed to include a family of hemagglutinin proteins, as well as non-hemagglutinin protein. The non-toxin proteins have been reported to stabilize the botulinum toxin molecule in the toxin complex and protect it against denaturation by digestive acids when the toxin complex is ingested. Thus, the non-toxin proteins of the toxin complex protect the activity of the toxin and thereby enhance systemic penetration when the toxin complex is administered via the gastrointestinal tract. Additionally, it is believed that some of the non-toxin proteins specifically stabilize the botulinum toxin molecule in blood.

The presence of non-toxin proteins in the toxin complexes typically causes the toxin complexes to have molecular weights that are greater than that of the bare botulinum toxin molecule. The molecular weight of botulinum toxin protein itself is about 150 kD, though the different serotype complexes vary in size. For example, C. botulinum bacteria can produce botulinum type A toxin complexes that have molecular weights of about 900 kD, 500 kD, or 300 kD. Botulinum toxin types B and C are produced as complexes having a molecular weight of about 700 kD or about 500 kD. Botulinum toxin type D is produced as complexes having molecular weights of about 300 kD or 500 kD. Botulinum toxin types E and F are only produced as complexes having a molecular weight of about 300 kD.

To provide additional stability to botulinum toxin, the toxin complexes are conventionally stabilized by combining the complexes with albumin during manufacturing. For example, BOTOX® (Allergan, Inc., Irvine, Calif.) is a botulinum toxin-containing formulation that contains 100 U of type A botulinum toxin with accessory proteins, 0.5 milligrams of human albumin, and 0.9 milligrams of sodium chloride.

Due to the molecule size and molecular structure of botulinum toxin, it does not on its own cross the stratum corneum of the skin and the multiple layers of the underlying skin architecture. Accordingly, the botulinum toxin typically is administered to patients by injection of compositions containing botulinum toxin complex and albumin. However, there are several problems associated with this approach. Not only are the injections painful, but typically large subdermal wells of toxin are locally generated around the injection sites, in order to achieve the desired therapeutic or cosmetic effect. The botulinum toxin may migrate from these subdermal wells to cause unwanted paralysis in surrounding areas of the body. This problem is exacerbated when the area to be treated is large and many injections of toxin are required to treat the area. Moreover, because the injected toxin complexes contain non-toxin proteins and albumin that stabilize the botulinum toxin and increase the molecular weight of the toxin complex, the toxin complexes have a long half-life in the body and may cause an undesirable antigenic response in the patient. For example, some patients will, over time, develop an allergic reaction to the albumin used as a stabilizer in current commercial formulations. Also, the toxin complexes may induce the immune system of the patient to form neutralizing antibodies, so that larger amounts of toxin are required in subsequent administrations to achieve the same effect. When this happens, subsequent injections must be carefully placed so that they do not release a large amount of toxin into the bloodstream of the patient, which could lead to fatal systemic poisoning.

As noted above, botulinum toxins have been used as injectable agents in the management of refractory plantar fasciitis. Nonetheless, about 10% of patients do not respond to these treatments within six to nine months. Many of the other current treatment options for plantar fasciitis also introduce additional problems. For example, steroid injections are frequently used by treating physicians, but side-effects can include atrophy of the fat pad in the foot, plantar fascia rupture, peripheral nerve injury, and muscle damage, as well as transient hyperglycemia (in diabetic patients). Extracorporeal therapy is often painful, requiring sedation or anesthesia, which increases expense.

In view of the drawbacks associated with current treatments and current botulinum toxin formulations, it would be highly desirable to have an injectable or transdermal botulinum toxin formulation that is efficacious and stable, but exhibits reduced antigenicity and a lower tendency to diffuse locally after injection. It would also be desirable to use such a botulinum toxin formulation for therapeutic purposes to treat plantar fasciitis, in particular, a stable, longer-acting treatment requiring fewer interventions.

### SUMMARY OF THE INVENTION

The invention relates to treatment and management of plantar fasciitis, or a disorder related thereto, using botulinum toxin compositions of the invention that can be administered by injection or transdermally to deliver a therapeutically effective amount of the compositions to a subject in need of such treatment. In particular, the injectable botulinum toxin formulations show therapeutic benefit in reducing plantar fasciitis pain with a surprisingly high responder rate and long duration of effect.

In one aspect, the compositions used are in sterile injectable formulations and administration is achieved by injection into one or more muscles or fascia associated with plantar fasciitis, in particular, one or more of the muscles or fascia causing pain associated with plantar fasciitis. In another aspect, botulinum toxin is administered in a transdermal formulation effective for delivering the botulinum toxin across the skin to the target areas for achieving a therapeutic effect. In methods of this invention, therapeutic effect comprises reducing, attenuating, or eliminating one or more symptoms of plantar fasciitis or related disorder. In particular embodiments, the symptom is pain associated with inflammation of the plantar fascia, and the therapeutic effect may be a reduction in the severity of the pain and/or a reduction in the frequency of the pain.

In particular embodiments, the invention provides a method of treating plantar fasciitis in an individual in need thereof, the method comprising administering to the individual a composition comprising: a carrier, a botulinum toxin component, and a pharmaceutically acceptable diluent for injection or topical application, where the carrier is non-covalently associated with the botulinum toxin component. In preferred embodiments, the botulinum toxin component comprises serotype A botulinum toxin having a molecular weight of 150 kDa. The botulinum toxin component may be selected from a botulinum toxin complex (including the 150 kD neurotoxin with accessory proteins found in native complexes produced by C. botulinum), a reduced botulinum toxin complex (including the 150 kD neurotoxin with some, but not all, of the native accessory proteins), and the 150 kD botulinum toxin molecule itself, without accessory proteins. In certain embodiments, the carrier is a positively charged carrier or a lipophilic carrier.

In another aspect, the invention provides topical or sterile injectable compositions comprising botulinum toxin non-covalently associated with the positively charged or lipophilic carrier for use in methods of treating or managing plantar fasciitis or a related disorder. In preferred embodiments, the compositions of the invention possess one or more advantages over conventional commercial botulinum toxin formulations, such as BOTOX® or MYOBLOC®. For instance, in certain embodiments, the injectable compositions exhibit one or more advantages over conventional injectable botulinum toxin formulations, including reduced antigenicity, a reduced tendency to undergo diffusion into surrounding tissue following injection, increased duration of clinical efficacy or enhanced potency relative to conventional botulinum toxin formulations, faster onset of clinical efficacy, and/or improved stability. In particularly preferred embodiments, the injectable compositions provide an attribute of reduced diffusion or spread from the injection site following injection, thereby localizing the toxin and its effect where desired and decreasing nonspecific or unwanted effects of the toxin at locations distant from the site of injection. In other particularly preferred embodiments, the topical compositions facilitate delivery of botulinum toxin transdermally to underlying target muscles. In some embodiments, the topical compositions are contained in a device for dispensing the botulinum toxin, where the device is applied topically to the skin, such as a skin patch. Topical approaches may be used instead of, or in conjunction with, injectable approaches.

According to the present invention, the positively charged or lipophilic carrier is suitable as a transport system for botulinum toxin, enabling the toxin to be injected or topically applied with improved characteristics, as discussed above, without covalent modification of the toxin molecule. The positively charged or lipophilic carrier comprises a positively charged or hydrophobic backbone, respectively, to which are covalently attached efficiency groups (also referred to as protein transduction domains (PTDs) or cell-penetrating peptides (CPPs)), more preferably at one or both ends of the backbone. In certain embodiments, the efficiency groups are amino acid sequences selected from the group consisting of HIV-TAT or fragments thereof; the PTD of Antennapedia or a fragment thereof; -(gly)ₙ₁-(arg)ₙ₂ (SEQ ID NO: 5) in which the subscript n1 is an integer of from 0 to about 20 and n2 is independently an odd integer from about 5 to about 25; or (gly)ₚ-RGRDDRRQRRR-(gly)_{q} (SEQ ID NO: 1), (gly)ₚ-YGRKKRRQRRR-(gly)_{q} (SEQ ID NO: 2), or (gly)ₚ-RKKRRQRRR-(gly)_{q} (SEQ ID NO: 3), wherein the subscripts p and q are each independently an integer of from 0 to about 20. In one particularly preferred embodiment, the positively charged carrier has the amino acid sequence RKKRRQRRRG-(K)₁₅-GRKKRRQRRR (SEQ ID NO: 4) (also referred to herein as “RTP004”). In still other embodiments, the positively charged carrier has the amino acid sequence YGRKKRRQRRR-G-(K)₁₅-G-YGRKKRRQRRR (SEQ ID NO: 7) or RGRDDRRQRRR-G-(K)₁₅-G-RGRDDRRQRRR (SEQ ID NO: 8). In alternate embodiments, the carrier is a lipophilic carrier comprising palmitoyl-GGRKKRRQRRR, palmitoyl-glyₚ-KKRPKPG, or oleyl-glyₚ-KKRPKPG, where p is an integer from 0 to 20.

In some embodiments, the carrier is provided in the botulinum toxin-containing composition in an amount from about 0.001 to about 1 μg per U of the botulinum toxin component, preferably about 0.01 to about 0.5 μg per U, more preferably about 0.05 to about 0.35 μg per U, or about 0.1 to about 0.3 μg per U, and most preferably about 0.234 μg per botulinum toxin unit. For example, the botulinum toxin-containing composition may contain about 10 to about 25 μg, about 12 to about 22 μg, about 15 to about 21 μg, or about 15 to about 20 μg of the carrier. In one preferred embodiment, the botulinum toxin is in a dosage amount selected from the group consisting of about 200 U to about 280 U, and the carrier is a positively charged carrier present in the composition in an amount selected from about 46 to about 66 μg, so as to provide a ratio of about 0.234 μg/U of botulinum toxin.

In some embodiments, the excipient of the botulinum toxin-containing composition comprises one or more additional stabilizing components selected from the group consisting of L-Histidine, L-Histidine hydrochloride, polysorbate 20, and trehalose dihydrate.

In some particularly preferred embodiments, the excipient comprises trehalose dihydrate. For example, per 50 U vial, the excipient may comprise about 1 mg to about 100 mg, about 10 to about 80 mg, about 20 mg to about 60 mg, about 30 mg to about 40 mg, or about 34 mg, about 35 mg, about 36 mg, about 37 mg, about 38 mg, about 39 mg, or about 40 mg trehalose. In some particularly preferred embodiments, the excipient comprises polysorbate 20. For example, per 50 U vial, the excipient may comprise about 0.01 mg to about 1 mg, about 0.05 to about 0.5 mg, about 0.075 mg to about 0.25 mg, about 0.08 mg to about 0.15 mg, or about 0.09 mg, about 0.095 mg, about 0.1 mg, about 0.105 mg, about 0.11 mg, about 0.12 mg, about 0.13 mg, about 0.14 mg, or about 0.15 mg polysorbate 20. In a particularly preferred embodiment, per 50 U, the excipient contains about 36 mg trehalose and about 0.1 mg polysorbate 20. In a more particularly preferred embodiment, a lyophilized formulation in a 50 U vial contains about 36 mg trehalose, about 0.1 mg polysorbate 20, and about 11.7 μg RTP004 as the carrier, to give a peptide carrier:toxin mass ratio of 51,000:1 in the 50 U vial.

In one particularly preferred embodiment, referred to as “RT002,” the composition is an injectable formulation, which contains the 150 kD subtype A botulinum toxin molecule, non-covalently associated with a positively charged carrier peptide having the formula RKKRRQRRRG-(K)₁₅-GRKKRRQRRR (SEQ ID NO: 4), and which does not contain accessory proteins or animal-derived components, and as described in WO 2010/151840 (PCT/US2010/040104) to Thompson et al., “Albumin-Free Botulinum Toxin Formulations.” See also, Garcia-Murray, “Safety and efficacy of RT002, an injectable botulinum toxin type A, for treating glabellar lines: results of a phase 1/2, open-label, sequential dose-escalation study” Dermatol Surg. 2015 January; 41 Suppl 1: S47-55; and Carruthers, et al., Injectable DaxibotulinumtoxinA for the Treatment of Glabellar Lines: A Phase 2, Randomized, Dose-Ranging, Double-Blind, Multicenter Comparison with Onabotulinumtoxin A and Placebo. Dermatol. Surg. 2017; 43: 1321-1331, describing the RT002 formulation; as well as WO 2017/075468 (PCT/US2016/059492) to Ruegg et al., entitled “Injectable Botulinum Toxin Formulations And Methods Of Use Thereof Having Long Duration Of Therapeutic Or Cosmetic Effect;” U.S. Provisional Patent Application No. 62/594,529, to Rubio, entitled “Injectable Botulinum Toxin Formulations and Methods of Use Thereof Having High Responder Rate and Long Duration of Effect,” filed Dec. 4, 2017; U.S. Provisional Patent Application No. 62/550,850, to Ruegg entitled “Transmucosal Botulinum Toxin Compositions, Kits, And Methods For Treating Bladder Disorders,” filed Aug. 28, 2017; and International Application PCT/US18/48361, to Ruegg entitled “Transmucosal Botulinum Toxin Compositions, Kits, And Methods For Treating Bladder Disorders,” filed Aug. 28, 2018, each incorporated-by-reference herein in its entirety. RT002 generally is provided in lyophilized form, in a 50 U vial of 150 kDa botulinum toxin A, containing 0.1 mg polysorbate 20, 36 mg trehalose, and 11.7 μg RTP004 as the carrier, to give a mass ratio of peptide:toxin of 51,000:1 in the 50 U vial.

Methods and compositions described herein deliver the botulinum toxin component in an amount effective to improve at least one symptom of plantar fasciitis or a disorder related thereto. In certain embodiments, the botulinum toxin is administered from about 1 U to about 1,000 U, preferably from about 100 U to about 500 U, more preferably from about 200 U to about 300 U; or more specifically, from about 220 U to about 280 U, from about 220 U to about 260 U, or about 240 U per injection treatment. In preferred embodiments, the botulinum toxin is in a dosage amount selected from the group consisting of about 200 U, about 220 U, about 240 U, about 260 U, about 280 U, and about 300 U.

In certain embodiments, the botulinum toxin is administered from about 1 U to about 1,000 U, preferably from about 20 U to about 200 U, more preferably from about 40 U to about 180 U; or more specifically, from about 50 U to about 160 U, from about 60 U to about 150 U, from about 70 U to about 130 U, or about 80 U to about 120 U per injection treatment. In preferred embodiments, the botulinum toxin is in a dosage amount selected from the group consisting of about 50 U, about 60 U, about 70 U, about 80 U, about 90 U, about 100 U, about 110 U, about 120 U, about 130 U, about 140 U, about 150 U, and about 160 U. In some such embodiments, the injection treatment is a single injection.

In particular embodiments, the composition is administered by injection into, or by topical application to skin overlying, one or more muscles or fascia associated with plantar fasciitis, in particular, one or more of the muscles or fascia causing pain associated with plantar fasciitis. Administration may comprise injection into one or more of muscles or fascia selected from the group consisting of plantar fascia (preferably the point of maximum tenderness in the plantar fascia), plantar spur (periosteum, preferably the periosteum over the plantar insertion), a short flexor, quadratus plantae, and triceps surae (gastrocnemium and soleus). In other embodiments, administration may comprise topical application to skin overlying one or more of the above-recited muscles and fascia. In more particular embodiments, specific dose amounts are injected into specific muscles or fascia; for example, in one embodiment, a dose of about 50 U to about 300 U, about 100 U to about 200 U, or about 160 U of the botulinum toxin component is injected into triceps sura; and a dose of about 10 U to about 150 U, about 50 U to about 100 U, or about 80 U of the botulinum toxin component is injected into at least one muscle or fascia selected from the group consisting of plantar fascia, plantar spur (periosteum), a short flexor, and quadratus plantae. In a particular example, a dose of about 50 U to about 300 U, about 100 U to about 200 U, or preferably about 160 U of the botulinum toxin component is injected into triceps sura, at about 2 cm intervals, e.g., at about 1, 2, 3, 4, 5, 6, 7, 8, 9, or 10 skin injection sites, preferably about 2-9, about 3-8, or about 4-7 sties, depending on the size of the subject's muscles; and/or a dose of about 10 U to about 150 U, about 50 U to about 100 U, or preferably about 80 U of the botulinum toxin component is injected into at least one muscle or fascia selected from the group consisting of plantar fascia (preferably a point of tenderness, more preferably a point of maximum tenderness), plantar spur (periosteum, preferably in the region of the plantar insertion, more preferably over the plantar insertion), a short flexor, and quadratus plantae, e.g., divided among about 1, 2, 3, 4, or 5 skin injection sites, preferably about 1-4 or about 1-3 sites, into one or more of the subject muscles or fascia.

In particular embodiments, administration may comprise injection into, or near to, one or more muscles or fascia selected from the group consisting of the plantar fascia, the flexor digitorum brevis, and the flexor halluces longus. In other embodiments, administration may comprise topical application to skin overlying one or more of the above-recited muscles and fascia. In particular embodiments, the injection treatment is a single injection through one site, preferably at one or more depths of administration. In more particular embodiments, specific dose amounts are injected into specific muscles or fascia at different depths of administration, via a single injection site. For example, in one embodiment, a single total dose is divided into two or more different fractions, which are administered in different regions. Ultrasound or other visualization methods may be used to guide the needle to the desired injection site. In one embodiment, a fraction of a dose of about 50 U to about 120 U, about 60 U to about 100 U, about 70 U to about 90 U, or about 80 U per injection of the botulinum toxin component is injected in the plantar fascia, preferably the plantar fascia at the medial calcaneal tuberosity; and the remainder of the dose of the botulinum toxin component is injected into or near to the region immediately superior to the plantar fascia, preferably at or near the flexor digitorum brevis and/or the flexor hallucis longus. For example, in another embodiment, a fraction of a dose of about 100 U to about 160 U, about 100 U to about 140 U, or about 120 U per injection of the botulinum toxin component is injected in the plantar fascia, preferably the plantar fascia at the medial calcaneal tuberosity; and the remainder of the dose of the botulinum toxin component is injected into or near to a region immediately superior to the plantar fascia, preferably at or near the flexor digitorum brevis and/or the flexor hallucis longus. The fraction for administration to the plantar fascia may be about ⅙, about ⅕, about ¼, about ⅓, about ½, about ⅔, or about ⅚ of the total injection dose, preferably about ⅓, with the remainder being about ⅚, about ⅘, about ¾, about ⅔, about ½, about ⅓, or about ⅙, preferably with the remainder being about ⅔ being administered

In another aspect, the invention provides a method of administering botulinum toxin to achieve an extended duration therapeutic effect in an individual suffering from plantar fasciitis or a disorder related thereto. In some embodiments, the method comprises administering by injection a dose of a sterile injectable composition into one or more muscles or fascia associated with plantar fasciitis to achieve the extended duration therapeutic effect following treatment, this is, sustained efficacy, or a response rate of long duration, following treatment. In some embodiments, the method comprises administering by topical application a dose of a topical composition to the skin overlying one or more muscles or fascia associated with plantar fasciitis to achieve the extended duration therapeutic effect following treatment. For example, in some embodiments, administration of the botulinum toxin compositions results in an increased duration of effect, such as an improvement in at least one symptom of plantar fasciitis that lasts longer than treatment with conventional botulinum toxin formulations, thereby extending treatment intervals. Preferred embodiments afford a reduction in one or more plantar fasciitis symptoms for at least about 3 months through about 11 months, about 5 months through about 10 months, about 6 months through about 10 months, or for at least about 20 weeks through about 40 weeks. In preferred embodiments, such as using a botulinum toxin dose of 240 U, preferably as in the RT002 composition, the duration of effect is at least about 16 weeks, at least about 20 weeks, at is at least about 24 weeks, at least about 26, weeks, at least about 28 weeks, at least about 30 weeks, at least about 32 weeks, at least about 34 weeks, at least about 36, weeks, at least about 40 weeks, or at least about 42 weeks, before a second or subsequent treatment dose is administered. In a particular embodiment, injection of the composition provides a single treatment dose that reduces plantar fasciitis pain by at least 50% 8 weeks following treatment.

In another aspect, the invention provides a method of treating an individual suffering from plantar fasciitis, where the method comprises a treatment course having multiple treatments with prolonged duration of effect and thus lengthier intervals between successive treatments compared to regimens using conventional botulinum toxin formulations. In particular embodiments, the interval before administering a second or subsequent treatment dose of the composition is greater than or equal to about 3 months, about 4 months, about 5 months, about 6 months, about 7 months, about 8 months, about 9 months, or greater than or equal to about 10 months, following the initial treatment dose or following subsequent treatment doses; or where the interval before administering a second or subsequent treatment dose of the composition is greater than or equal to about 26 weeks, about 28 weeks, about 30 weeks, about 32 weeks, about 34 weeks, about 36 weeks, about 38 weeks, about 40 weeks, or greater than or equal to about 42 weeks, following the initial treatment dose or following subsequent treatment doses.

The methods of treatment achieve surprisingly long duration and high responder rates. In particular embodiments, the invention provides methods of improving at least one symptom of plantar fasciitis or a disorder related thereto for an individual in need thereof, the method comprising: administering to the individual by injection to one or more muscles or fascia associated with plantar fasciitis pain a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO; 4); wherein the botulinum toxin component is administered to the individual in a treatment dose amount of about 50 U to about 300 U, about 200 U to about 300 U, about 60 to about 160 U, or about 80 U, about 100 U, about 120 U, or about 240 U in a single injection; wherein the positively charged carrier is non-covalently associated with the botulinum component; and wherein the injection of the composition provides a single treatment dose having at least about a 26-week duration of effect in reducing at least one symptom of plantar fasciitis for the individual, thereby extending treatment interval duration for the individual. The symptom preferably is a reduction in pain typically associated with plantar fasciitis.

Accordingly, in some embodiments, the methods and uses of the pharmaceutical composition, as described above, allow for methods of treating plantar fasciitis in an individual in need thereof with injectable botulinum toxin, wherein the method comprises a treatment course having multiple treatment intervals with prolonged duration of effect, and duration of treatment intervals, the treatment course comprising: administering by injection an initial treatment dose of a sterile injectable composition into one or more muscles or fascia of the individual associated with plantar fasciitis pain, to achieve a reduction in the pain following the initial treatment with the composition; wherein the composition comprises a pharmaceutically acceptable diluent suitable for injection; a botulinum toxin component that is botulinum toxin of serotype A having a molecular weight of 150 kDa without accessory non-toxin proteins; and a positively charged carrier having the amino acid sequence RKKRRQRRRG-(K)₁₅-GRKKRRQRRR (SEQ ID NO: 4); wherein the botulinum toxin component is administered to the individual in a treatment dose of about 50 U to about 200 U, about 60 U to about 160 U, or about 80 U, about 100 U, about 120 U, or about 240 U in a single injection; wherein the positively charged carrier is non-covalently associated with the botulinum toxin component; wherein the initial treatment dose of the composition administered by injection to the individual provides a duration of effect lasting through at least about 20-26 weeks; and administering subsequent treatment doses of the composition by injection to the individual at treatment intervals comprising a duration of greater than or equal to about 20 weeks to at least about 40 weeks following the initial treatment dose and between each subsequent treatment dose.

In some embodiments of the methods and uses described in the above two paragraphs, the composition achieves an extended duration of effect for at least about 27 weeks, at least about 28 weeks, at least about 29 weeks, or at least about 30 weeks. In some such embodiments, the positively charged carrier is present in said pharmaceutical composition in an amount of about 0.1 to about 0.3 μg per unit of botulinum toxin component, preferably in an amount of about 0.234 μg per unit of botulinum toxin component. Alternatively or in addition, in some such embodiments, the excipient comprises at least one component selected from the group consisting of L-Histidine, L-Histidine hydrochloride, polysorbate 20, and trehalose dihydrate, preferably trehalose dihydrate.

In preferred embodiments, the effect is a reduction in pain associated with plantar fasciitis. In such embodiments, administration may comprise at least one injection into one or more muscles or fascia selected from the group consisting of the plantar fascia, plantar spur (periosteum), a short flexor, quadratus plantae, and triceps surae (gastrocnemium and soleus). For example, in some of these embodiments, about 160 U of the botulinum toxin component are injected into the gastrocnemium-soleus complex; and about 80 U of the botulinum toxin component are injected into the plantar fascia (preferably at the point of maximum tenderness), plantar spur (preferably the periosteum over the plantar insertion), quadratus plantae, and/or a short flexor. In other such embodiments, administration may comprise a single injection through one site, administering a third of a dose of about 50 U to about 200 U, about 60 U to about 160 U, or a third of about 80 U, a third of about 100 U, or a third of about 120 U, in a single injection to the plantar fascia, preferably the plantar fascia at the medial calcaneal tuberosity; and the remainder two thirds of the dose to or near to a region immediately superior to the plantar fascia, preferably at or near the flexor digitorum brevis and/or the flexor hallucis longus.

The duration of effect provided by compositions of the invention, and methods and uses thereof, affords significant advantages compared to the art. By way of example, subjects undergoing treatment with compositions containing botulinum toxin consider duration of effect to be of high importance. A long, sustained duration of effect, which can be achieved by even a single treatment with an effective dose according to the invention, permits fewer injections or topical applications per treatment course for a subject. For example, a prolonged duration of effect from a single injection treatment with a product having clear efficacy and safety, as provided by the inventive compositions and methods herein, offers less discomfort, less cost, and more convenience to subjects undergoing treatments. Furthermore, a prolonged duration of effect from a single treatment with a topically-applied product even further reduces discomfort and even further improves convenience. Such prolonged duration of action permits fewer treatments over an entire treatment course. Moreover, in some preferred embodiments, treatment regimens provided herein achieve sustained relief from chronic heel pain and can support healing of the plantar fascia, with reduced risks of plantar fascia rupture and/or atrophy of the fat pad. Accordingly, a product that affords safe, significant, and sustained effect, following a single injection treatment or topical application, provides a solution to an unmet need in the art for both practitioners and patients. Thus, the compositions and methods of the invention provide a solution to the problem of too frequent, painful, and/or inconvenient treatments, thereby improving overall well-being of the plantar fasciitis patient.

In another aspect, the invention provides for methods of treating plantar fasciitis in an individual with higher responder rates compared with conventional botulinum toxin formulations. In some embodiments, the invention provides for methods of treating plantar fasciitis in an individual in need thereof with injectable botulinum toxin, the method comprising: administering to the individual by injection to one or more muscles or fascia associated with plantar fasciitis a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 200 U to about 300 U, or about 240 U per injection treatment; or about 50 U to about 200 U, about 60 to about 160 U, or about 80 U, about 100 U, or about 120 U, in a single injection to or near to the plantar fascia; wherein the positively charged carrier is non-covalently associated with the botulinum component; and wherein the injection of the composition provides an effect of reducing plantar fasciitis pain with an increased rate of response for individuals, each administered the pharmaceutical composition, compared to individuals administered conventional botulinum toxin formulations.

In preferred such embodiments, the effect endures for at least about 4 weeks in over 55%, preferably over 60%, more preferably over 70% of individuals each administered the pharmaceutical composition. In more preferred embodiments, the effect endures for at least about 16 weeks in over 35%, preferably over 50%, more preferably over 70%, of individuals each administered the pharmaceutical composition. In even more preferred embodiments, the effect endures for at least about 24 weeks in over 15%, most preferably over 25%, of individuals each administered the pharmaceutical composition.

This invention also provides kits for preparing formulations containing a botulinum toxin, a botulinum toxin complex, or a reduced botulinum toxin complex and positively charged carrier, or a premix that may in turn be used to produce such a formulation. Also provided are kits that contain means for simultaneously or sequentially administering the botulinum toxin component and the positively charged carrier.

### BRIEF DESCRIPTION OF THE FIGURES

FIG. 1 depicts a schema of the protocol used in Example 1, describing a prospective, randomized, double-blinded, placebo-controlled clinical trial of an injectable formulation of the invention (referred to “RT002” or “Daxibotulinumtoxin A for injection”), comprising 150 kDa botulinum toxin type A in association with the peptide carrier, RKKRRQRRRG-(K)₁₅-GRKKRRQRRR (SEQ ID NO: 4).

FIG. 2 depicts a schedule of assessments for use in accordance with the schema of FIG. 1.

FIG. 3 depicts anatomical structures typically involved in plantar fasciitis.

FIG. 4 depicts an overview of a Phase 2a prospective, randomized, double-blind, placebo-controlled trial, evaluating RT002 efficacy and safety in managing plantar fasciitis, following subjects over 16 weeks across five U.S. sites.

FIG. 5 depicts primary endpoint results using the Visual Analog Scale (VAS) for pain scores at Week 8; results were based on data from an intent-to-treat (ITT) population analyzed by an Analysis of Covariate (ANCOVA) model adjusting for center and baseline VAS scores with the last-observation-carried-forward (LOCF) approach.

FIG. 6 depicts secondary endpoint results for change in VAS for pain scores over time, where the reduction in VAS scores observed beginning at Week 1 continued through Week 8 for both test and placebo treatment groups; results were based on only observed data for subjects in the intent-to-treat (ITT) population.

FIG. 7 depicts secondary endpoint results for change in VAS for pain scores over time, based on only observed data for subjects in the intent-to-treat (ITT) population, and further compared with results using other botulinum toxin formulations.

FIG. 8 depicts secondary endpoint results for change in VAS for pain scores over time, based on only observed data for subjects in the intent-to-treat (ITT) population, and further compared with results using other botulinum toxin formulations and steroids.

FIGS. 9A-9D depict efficacy endpoints at Week 8, including primary endpoint based on reduction in VAS for pain (FIG. 9A), secondary endpoints based on improvement in AOFAS score (FIG. 9B) and in FADI score (FIG. 9C), and exploratory endpoint based on reduction in PFPS score (FIG. 9D); results were based on data for subjects in the intent-to-treat (ITT) population analyzed by an ANCOVA model adjusting for center and baseline VAS scores with the last-observation-carried-forward (LOCF) approach.

FIG. 10 depicts secondary endpoint results for change in AOFAS over time, based on only observed data for subjects in the intent-to-treat (ITT) population.

FIG. 11 depicts secondary endpoint results for change in AOFAS over time, based on observed data for subjects in the intent-to-treat (ITT) population, and further compared with results using other botulinum toxin formulations and steroids.

FIG. 12 depicts secondary endpoint results for change in FADI over time, based on only observed data for subjects in the intent-to-treat (ITT) population.

FIG. 13 depicts secondary endpoint results for change in FADI over time, based on observed data for subjects in the intent-to-treat (ITT) population, and further compared with results using other botulinum toxin formulations.

FIG. 14 depicts results of two sensitivity analyses performed to assess impact of analgesia (Group [a]) and anti-inflammatory medication (Group [b]) on VAS for pain outcome measure at Week 8; results were based on data for subjects in the intent-to-treat (ITT) population analyzed by an ANCOVA model adjusting for center and baseline VAS scores with the last-observation-carried-forward (LOCF) approach.

FIGS. 15A-15E depict VAS pain scores over time by study center, in each of the five study centers of this trial, based on only observed data for subjects in the intent-to-treat (ITT) population.

FIG. 16 depicts results of a further sensitivity analysis of primary endpoint results for VAS for pain scores at Week 8, excluding results from one study center (Group [c]); results were based on data from an intent-to-treat (ITT) population analyzed by an ANCOVA model adjusting for study center and baseline VAS scores with the last-observation-carried-forward (LOCF) approach.

FIG. 17 depicts results of a sensitivity analysis of secondary endpoint results for change in VAS for pain scores over time, excluding results from one study center (Group [c]); results were based on only observed data for subjects in an intent-to-treat (ITT) population.

FIG. 18A and FIG. 18B depicts a schema of the protocol used in Example 3, describing a prospective, randomized, double-blinded, placebo-controlled clinical trial of RT002 injected only at the plantar fascia.

FIG. 19 depicts a schedule of assessments for use in accordance with the schema of FIGS. 18A-18B.

The patent or application file contains at least one drawing executed in color. Copies of this patent or patent application publication with color drawing(s) will be provided by the Office upon request and payment of the necessary fee.

### DETAILED DESCRIPTION OF THE INVENTION

This invention relates to botulinum toxin-containing compositions for use in the treatment or management of plantar fasciitis or a disorder related thereto. In one aspect, the compositions used are in sterile injectable formulations that can be administered to an individual with plantar fasciitis by injection, such as by injection into one or more muscles or fascia associated with the plantar fasciitis to achieve a therapeutic effect. In another aspect, the compositions used are in transdermal (or topical) formulations that can be administered to an individual with plantar fasciitis by topical application to skin overlying muscles and fascia, where the botulinum toxin is delivered across the skin to the target areas for achieving a therapeutic effect. Generally, the invention provides methods of treating plantar fasciitis by administering by injection or transdermally, to an individual in need thereof, a therapeutically effective amount of a composition comprising: a botulinum toxin component, a carrier, and a pharmaceutically acceptable diluent for injection or topical application, where the carrier is non-covalently associated with the botulinum toxin component.

In particular, this invention relates to botulinum toxin-containing compositions for use in the producing higher responder rates and/or longer duration of effect in therapeutic use of botulinum toxin for plantar fasciitis. That is, certain aspects of the invention relate to botulinum toxin-containing compositions for use in producing higher responder rates in patients with plantar fasciitis over an extended period of time compared with commercially available botulinum toxin preparations, such as BOTOX®. In certain embodiments, treatment results in about 30%, about 40%, about 50%, about 60%, or about 70% reduction in pain, preferably as measured by the VAS or the NPRS for pain, and this pain reduction lasts through weeks one, two, three, four, five, six, seven, eight, nine, ten, eleven, twelve, thirteen, fourteen, fifteen, or sixteen, following treatment. In a particular embodiment, plantar fasciitis pain is reduced by 50%, or more, through week eight following a single injection treatment, and preferably in a single injection, such as a single injection through one site through one site. The single injection may be distributed in one or more different fractions. In one embodiment the injection is divided into two different fractions. Ultrasound or other means may be used to guide the injection.

In particular embodiments, the invention provides methods of reducing plantar fasciitis pain in an individual in need thereof, the method comprising: administering to the individual by injection to one or more muscles or fascia associated with plantar fasciitis pain, a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 200 U to about 300 U, preferably about 240 U per injection treatment; or about 50 U to about 200 U, about 60 to about 160 U, or about 80 U, about 100 U, or about 120 U, in a single injection to or near to the plantar fascia; wherein the positively charged carrier is non-covalently associated with the botulinum component; and wherein the injection of the composition provides a single treatment dose having at least about a 20- to 40-week duration of effect in reducing at least one symptom of plantar fasciitis in the individual, thereby extending treatment interval duration for the individual. The symptom is preferably pain associated with plantar fasciitis.

Accordingly, the methods and uses of the pharmaceutical composition, as described above, allows for methods of treating plantar fasciitis in an individual in need thereof with injectable botulinum toxin, wherein the method comprises a treatment course having multiple treatment intervals with prolonged duration of effect, and duration of treatment intervals, the treatment course comprising: administering by injection an initial treatment dose of a sterile injectable composition into the individual's muscles and/or fascia associated with plantar fasciitis, in particular, the plantar fascia, to achieve a reduction in at least one symptom of plantar fasciitis following the initial treatment with the composition; wherein the composition comprises a pharmaceutically acceptable diluent suitable for injection; a botulinum toxin component that is botulinum toxin of serotype A having a molecular weight of 150 kDa without accessory non-toxin proteins; and a positively charged carrier having the amino acid sequence RKKRRQRRRG-(K)₁₅-GRKKRRQRRR (SEQ ID NO: 4); wherein the botulinum toxin component is administered to the individual in a treatment dose of about 200 U to about 300 U, preferably about 240 U per injection treatment; or about 50 U to about 200 U, about 60 to about 160 U, or about 80 U, about 100 U, or about 120 U, in a single injection to or near to the plantar fascia; wherein the positively charged carrier is non-covalently associated with the botulinum toxin component; wherein the initial treatment dose of the composition administered by injection to the individual provides a duration of effect lasting through at least about 20-26 weeks; and administering subsequent treatment doses of the composition by injection to the individual at treatment intervals comprising a duration of greater than or equal to about 20 weeks to at least about 40 weeks following the initial treatment dose and between each subsequent treatment dose.

Along with extended duration of effect, it also surprisingly been found that the effect occurs in a higher proportion of individuals receiving treatment compared with commercially available botulinum toxin preparations, such as BOTOX®. In some embodiments of any of the above methods or uses, the reduction in a symptom of plantar fasciitis, such as a reduction in pain, endures for at least about 4 weeks in over 55% of individuals each administered the pharmaceutical composition, preferably over 60%, more preferably over 70% of individuals each administered the pharmaceutical composition. In some embodiments of any of the above methods or uses, the reduction in a symptom of plantar fasciitis, such as a reduction in pain, endures for at least about 16 weeks in over 35%, preferably over 50%, more preferably over 70%, of individuals each administered the pharmaceutical composition. In some embodiments of any of the above methods or uses, the reduction in a symptom of plantar fasciitis, such as a reduction in pain, endures for at least about 24 weeks in over 15%, preferably over 25%, of individuals each administered the pharmaceutical composition.

Thus, methods and uses, as described above, allow for methods of treating plantar fasciitis in an individual in need thereof with injectable botulinum toxin, the method comprising: administering to the individual by injection to, or near to, one or more muscles or fascia associated with the plantar fasciitis (such as the plantar fascia, gastrocnemius-soleus complex, periosteum, quadratus plantae, short flexors, flexor digitorum brevis, and flexor hallucis longus) a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 200 U to about 300 U, preferably about 240 U per injection treatment; or in a treatment dose amount of about 50 U to about 200 U, about 60 to about 160 U, or about 80 U, about 100 U, or about 120 U in a single injection; wherein the positively charged carrier is non-covalently associated with the botulinum component; and wherein the injection of the composition provides an effect of reducing at least one symptom of the plantar fasciitis, such as reducing plantar fasciitis pain, with an increased rate of response for individuals, each administered the pharmaceutical composition, compared to individuals administered conventional botulinum toxin formulations.

In preferred embodiments, the effect endures for at least about 4 weeks in over 55%, over 56%, over 58%, over 60%, over 62%, over 65%, over 66%, over 68%, over 70%, over 72%, over 73%, or over 75% of individuals each administered the pharmaceutical composition. In more preferred embodiments, the effect endures for at least about 16 weeks in over 35%, over 36%, over 38%, over 40%, over 43%, over 45%, over 47%, over 50%, over 53%, over 55%, over 57%, over 60%, over 63%, over 65%, over 68%, more preferably over 70%, over 73%, or over 75%, of individuals each administered the pharmaceutical composition. In even more preferred embodiments, the effect endures for at least about 24 weeks in over 15%, over 16%, over 18%, over 20%, over 22%, over 23%, over 25%, over 27%, or over 30%, of individuals each administered the pharmaceutical composition.

As used herein, the terms compositions and formulations are essentially interchangeable when referring to the compositions and formulations according to the present invention.

Injectable and Topical Compositions

Injectable compositions of this invention, in preferred embodiments, stabilize the toxin and/or enable its delivery through tissues after injection, such that the toxin has reduced antigenicity, a better safety profile, enhanced potency, faster onset of clinical efficacy, and/or longer duration of clinical efficacy compared to conventional commercial botulinum toxin formulations (e.g., BOTOX® or MYOBLOC®). In particularly preferred embodiments, the injectable compositions provide an attribute of reduced diffusion or spread from the injection site following injection, thereby localizing the toxin and its effect where desired and decreasing nonspecific or unwanted effects of the toxin at locations distant from the site of injection. The injectable compositions comprise a botulinum toxin in non-covalent association with an effective amount of a positively charged carrier, the carrier comprising a positively charged backbone with covalently attached positively charged “efficiency groups,” which also are referred to as protein transduction domains (PTDs) or cell-penetrating peptides (CPPs).

Topical compositions of this invention enable transport or delivery of botulinum toxin through the skin, allowing the toxin molecule to penetrate layers of skin impermeable to botulinum toxin formulations lacking carriers described herein. The topical compositions comprise a botulinum toxin in non-covalent association with an effective amount of the carrier, which can be a lipophilic carrier or a positively charged carrier. Lipophilic carriers comprise a hydrophobic backbone to which is covalently attached positively charged efficiency groups; positively charged carriers comprise a positively charged backbone, to which is covalently attached positively charged efficiency groups.

According to the present invention, the positively charged or lipophilic carrier is suitable as a transport system for botulinum toxin, enabling the toxin to be injected or topically applied with improved characteristics, as discussed above, without covalent modification of the toxin molecule.

The following sections describe the various components of the compositions for use in the present invention.

The Botulinum Toxin Component

The term “botulinum toxin” as used herein may refer to any of the known types of botulinum toxin (e.g., 150 kD botulinum toxin protein molecules associated with the different serotypes of C. botulinum), whether produced by the bacterium or by recombinant techniques, as well as any types that may be subsequently discovered including newly discovered serotypes, and engineered variants or fusion proteins. As mentioned above, currently seven immunologically distinct botulinum neurotoxins have been characterized, namely botulinum neurotoxin serotypes A, B, C1, D, E, F and G, each of which is distinguished by neutralization with type-specific antibodies. The different serotypes of botulinum toxin vary in the animal species that they affect and in the severity and duration of the paralysis they evoke. In preferred embodiments, the composition comprises a botulinum toxin of serotype A.

The botulinum toxin serotypes are commercially available, for example, from Sigma-Aldrich (St. Louis, Mo.) and from Metabiologics, Inc. (Madison, Wis.), as well as from other sources. At least two types of botulinum toxin, types A and B, are available commercially in formulations for treatment of certain conditions. Type A, for example, is contained in preparations of Allergan, Inc., having the trademark BOTOX®, as well as in preparations of Ipsen Limited, having the trademark DYSPORT®. The original Botox® formulation was prepared by Schantz in 1979 (Schantz et al., “Preparation and characterization of botulinum toxin type A for human treatment” Therapy with Botulinum Toxin. Vol. 109. New York, N.Y.: Marcel Dekker; 1994. pp. 10-24). Type B is contained, for example, in preparations of Elan Pharmaceuticals having the trademark MYOBLOC®. Recombinant botulinum toxin can also be purchased, e.g., from List Biological Laboratories, Campbell, Calif.

The term “botulinum toxin” can alternatively refer to a botulinum toxin derivative, that is, a compound that has botulinum toxin activity but contains one or more chemical or functional alterations on any part or on any amino acid chain relative to naturally occurring or recombinant native botulinum toxins. For instance, the botulinum toxin may be a modified neurotoxin that is a neurotoxin which has at least one of its amino acids deleted, modified, or replaced, as compared to a native form, or the modified neurotoxin can be a recombinantly produced neurotoxin or a derivative or fragment thereof. For instance, the botulinum toxin may be one that has been modified in a way that, for instance, enhances its properties or decreases undesirable side effects, but that still retains the desired botulinum toxin activity. Alternatively the botulinum toxin used in this invention may be a toxin prepared using recombinant or synthetic chemical techniques, e.g., a recombinant peptide, a fusion protein, or a hybrid neurotoxin, for example prepared from subunits or domains of different botulinum toxin serotypes (See, U.S. Pat. No. 6,444,209, for instance). The botulinum toxin may also be a portion of the overall molecule that has been shown to possess the necessary botulinum toxin activity and, in such case, may be used per se or as part of a combination or conjugate molecule, for instance a fusion protein. Alternatively, the botulinum toxin may be in the form of a botulinum toxin precursor, which may itself be non-toxic, for instance a non-toxic zinc protease that becomes toxic on proteolytic cleavage.

The term “botulinum toxin complex,” or “toxin complex,” as used herein refers to the approximately 150 kD botulinum toxin protein molecule (belonging to any one of botulinum toxin serotypes A-G), along with associated endogenous non-toxin proteins (i.e., hemagglutinin protein and non-toxin non-hemagglutinin protein produced by C. botulinum bacteria). In some embodiments, the botulinum toxin complex need not be derived from C. botulinum bacteria as one unitary toxin complex, but rather may be, for example, botulinum toxin that is recombinantly prepared first and then subsequently combined with the non-toxin proteins.

The term “reduced botulinum toxin complex,” or “reduced toxin complex,” refers to botulinum toxin complexes having reduced amounts of non-toxin protein compared to the amounts naturally found in botulinum toxin complexes produced by C. botulinum bacteria. In one embodiment, reduced botulinum toxin complexes are prepared using any conventional protein separation method to extract a fraction of the hemagglutinin protein or non-toxin non-hemagglutinin protein from botulinum toxin complexes derived from C. botulinum bacteria. For example, reduced botulinum toxin complexes may be produced by dissociating botulinum toxin complexes through exposure to red blood cells at a pH of 7.3, HPLC, dialysis, columns, centrifugation, and other methods for extracting proteins from complexes. Other procedures that can be used are described in, e.g., U.S. Pat. No. 9,469,849 to Ruegg, entitled “Methods And Systems For Purifying Non-Complexed Botulinum Neurotoxin;” WO 2006/096163 to Allergan, Inc., entitled “Animal Product Free System And Process For Purifying A Botulinum Toxin;” and EP 1514556 B1, to Allergan, Inc., entitled “Botulinum toxin pharmaceutical compositions,” each hereby incorporated herein by reference in its entirety. Alternatively, when the reduced botulinum toxin complexes are to be produced by combining synthetically produced botulinum toxin with non-toxin proteins, one may simply add less hemagglutinin or non-toxin, non-hemagglutinin protein to the mixture than what would be present for naturally occurring botulinum toxin complexes.

Any of the non-toxin proteins (e.g., hemagglutinin protein or non-toxin non-hemagglutinin protein or both) in the reduced botulinum toxin complexes may be reduced independently, by any amount. For example, although the amount of endogenous non-toxin proteins may be reduced by the same amount in some cases, this invention also contemplates reducing each of the endogenous non-toxin proteins by different amounts, as well as reducing at least one of the endogenous non-toxin proteins, but not the others.

In certain exemplary embodiments, one or more non-toxin proteins are reduced by at least about 0.5%, 1%, 3%, 5%, 10%, 20%, 30%, 40%, 50%, 60%, 70%, 80%, 90% or 100% compared to the amounts normally found in botulinum toxin complexes. As noted above, C. botulinum bacteria produce seven different serotypes of toxin. Commercial preparations are manufactured with different relative amounts of non-toxin proteins. For example, MYOBLOC® has 5000 U of Botulinum toxin type B per ml with 0.05% human serum albumin, 0.01 M sodium succinate, and 0.1 M sodium chloride. DYSPORT® has 500 U of botulinum toxin type A-hemagglutinin complex with 125 μg albumin and 2.4 mg lactose. In certain embodiments, substantially all of the non-toxin protein (e.g., greater than 95%, 96%, 97%, 98% or 99% of the hemagglutinin protein and non-toxin non-hemagglutinin protein) that would normally be found in botulinum toxin complexes derived from C. botulinum bacteria is removed from the botulinum toxin complex.

Accordingly, in various embodiments, the botulinum toxin component of the present compositions can be selected from a botulinum toxin complex (including the 150 kD neurotoxin with accessory proteins found in native complexes produced by C. botulinum bacteria, as described above), a reduced botulinum toxin complex (including the 150 kD neurotoxin with some, but not all, of the native accessory proteins), and the 150 kD botulinum toxin molecule itself, without accessory proteins.

In the present composition, botulinum toxin non-covalently associates with a carrier to form a complex without covalent modification to the botulinum toxin molecule. The association between the carrier and the botulinum toxin involves one or more types of non-covalent interaction, non-limiting examples of which include ionic interactions, hydrogen bonding, van der Waals forces, or combinations thereof. See also, e.g., WO 2005/084410 (PCT/US2005/007524), to Dake et al., “Compositions and Methods for Topical Application and Transdermal Delivery of Botulinum Toxins,” further describing how non-covalent association avoids the need to covalently modify the toxin molecule being delivered. The carrier molecules for use in the compositions are described below.

Carrier Molecules

According to the present invention, a positively charged or lipophilic carrier molecule, having covalently attached efficiency groups, as described herein, is suitable as a transport system for botulinum toxin in the treatment and management of plantar fasciitis or disorder related thereto. In certain embodiments, the positively charged or lipophilic carrier will not have other enzymatic or therapeutic biologic activity.

In injectable compositions, positively charged carriers enable toxin to be injected with improved delivery to target structures, resulting in decreased diffusion away from injected muscles or fascia, such as one or more muscles associated with plantar fasciitis. Besides enhancing delivery of botulinum toxin, the positively charged carriers may, in certain preferred embodiments, stabilize the botulinum toxin against degradation. In such embodiments, the hemagglutinin protein and non-toxin, non-hemagglutinin protein that are normally present to stabilize botulinum toxin may be reduced or omitted entirely, for example, as described above. Similarly, the exogenous albumin that is normally added during manufacturing may be omitted.

In alternate embodiments, in transdermal compositions, a positively charged or lipophilic carrier has the effect of promoting translocation of botulinum toxin through a tissue or cell membrane, such as through the skin overlying one or more structures associated with plantar fasciitis. The translocation occurs without covalent modification of the botulinum toxin. In certain embodiments, the positively charged or lipophilic carrier is the sole agent necessary for transdermal delivery of the botulinum toxin.

Exemplary positively charged carriers that can be used in injectable or topical compositions of the invention are described, e.g., in WO 2002/007773 (PCT/US2001/023072) to Waugh et al., “Multi-Component Biological Transport Systems;” WO 2005/084410 (PCT/US2005/007524), to Dake et al., “Compositions and Methods for Topical Application and Transdermal Delivery of Botulinum Toxins;” WO 2010/151840 (PCT/US2010/040104) to Thompson et al., “Albumin-Free Botulinum Toxin Formulations”; WO 2009/015385 (PCT/US2008/071350) to Stone et al., “Antimicrobial Peptide, Compositions, and Methods of Use;” WO 2013/112974 (PCT/US2013/023343) to Waugh et al., “Methods and Assessment Scales for Measuring Wrinkle Severity;” and/or WO 2014/066916 (PCT/US2013/67154) to Ruegg et al. “Compositions and Methods for Safe Treatment of Rhinitis;” exemplary lipophilic carriers that may be used in topical compositions of the present invention are described, e.g., in US 2016/0166703 A1 to Tan et al., entitled “Carrier Molecule Compositions and Related Methods” and in US 2014/0056811 A1 to Jacob, et al., entitled “New Cell-Penetrating Peptides And Uses Thereof,” each of which is incorporated herein by reference in their entireties.

By the use of the terms “positively charged” or “cationic,” it is meant that the carrier has a positive charge under at least some solution-phase conditions, more preferably, under at least some physiologically compatible conditions. More specifically, “positively charged” or “cationic” means that the group in question contains functionalities that are charged under physiological pH conditions, for instance, a quaternary amine, or that the group contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. Generally, the positively charged carrier comprises a positively charged backbone, described in more detail below.

Positively Charged Backbones of the Carrier Molecules

The positively charged backbone typically is a chain of atoms, either with groups in the chain carrying a positive charge at physiological pH, or with groups carrying a positive charge attached to side-chains. Generally, the backbone is a linear hydrocarbon backbone which is, in some embodiments, interrupted by heteroatoms selected from nitrogen, oxygen, sulfur, silicon, and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) and can be a homopolymer or a heteropolymer.

In certain preferred embodiments, the positively charged backbone comprises a cationic peptide, such as a polypeptide having multiple positively charged sidechain groups (e.g., lysine, arginine, ornithine, homoarginine, and the like). One of skill in the art will appreciate that when amino acids are used in this portion of the invention, the sidechains can have either the D- or L-form (R or S configuration) at the center of attachment.

As used herein, the term “peptide” refers to an amino acid sequence, but carries no connotation with respect to the number of amino acid residues within the amino acid sequence. Accordingly, the term “peptide” may also encompass polypeptides and proteins. For example, cationic peptide backbones of the invention may comprise from about 5 to about 100 amino acid residues, from about 10 to about 50 amino acid residues, or from about 12 to about 20 amino acid residues. In preferred embodiments, the cationic peptide backbone comprises 10 to 20 amino acids, or 10, 11, 12, 13, 14, 15, 16, 17, 18, 19, or 20 amino acids, preferably being polylysine amino acid residues.

In particularly preferred embodiments, the positively charged backbone is a polylysine. In some embodiments, the polylysine may have a molecular weight that is at least about 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention. The polylysine contemplated by this invention can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000.

In some preferred embodiments, the polylysine has a molecular weight from about 1,000 to about 1,500,000 D, from about 2,000 to about 800,000 D, or from about 3,000 to about 200,000 D. In more preferred embodiments, the polylysine has molecular weight from about 100 to about 10,000 D, from about 500 to about 5,000 D, from about 1,000 to about 4,000 D, from about 1,500 to about 3,500 D, or from about 2,000 to about 3,000 D. Especially preferred is a polylysine polypeptide having 10 to 20 lysines (SEQ ID NO: 9), more preferably, 15 lysines. The selection of an appropriate polylysine will depend on the remaining components of the composition and will be sufficient to provide an overall net positive charge to a positively charged carrier.

In another embodiment, the positively charged backbone is a nonpeptidyl polymer, which may be a hetero- or homo-polymer such as a polyalkyleneimine, for example a polyethyleneimine or polypropyleneimine. In some embodiments, the positively charged backbone is a polypropyleneamine wherein a number of the amine nitrogen atoms are present as ammonium groups (tetra-substituted) carrying a positive charge. In another group of embodiments, the backbone has attached a plurality of side-chain moieties that include positively charged groups (e.g., ammonium groups, pyridinium groups, phosphonium groups, sulfonium groups, guanidinium groups, or amidinium groups).

Alternatively, the backbone may comprise amino acid analogs and/or synthetic amino acids. The backbone may also be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the alpha-carbon atoms. As above, a portion or all of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the alpha-carbon locations.

A variety of other backbones can be used employing, for example, steric or electronic mimics of polypeptides wherein the amide linkages of the peptide are replaced with surrogates, such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene fluoroalkene (—CF═CH—), dimethylene (—CH₂CH₂—), thioether hydroxyethylene (—CH(OH)CH₂—), methyleneoxy tetrazole (CN), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from alpha-amino acids.

When the carrier comprises a relatively short linear polylysine or PEI backbone, the backbone will have a molecular weight of less than 75,000 D, more preferably less than 30,000 D, and most preferably, less than 25,000 D. When the carrier is a relatively short branched polylysine or PEI backbone, however, the backbone will have a molecular weight less than 60,000 D, more preferably less than 55,000 D, and most preferably less than 50,000 D.

In one particularly preferred embodiment, the carrier comprises a relatively short polylysine or polyethyleneimine (PEI) backbone (which may be linear or branched) and which has positively charged efficiency groups covalently attached. In more particularly preferred embodiments, the positively charged backbone is a polylysine and positively charged efficiency groups are attached to the lysine at the C- and/or N termini. The efficiency groups are described in detail below.

Efficiency Groups

Generally, the positively charged or hydrophobic backbone, has covalently attached one or more efficiency groups (PTDs or CPPs). The efficiency groups can be placed at spacings along the backbone that are consistent in separations or variable. In preferred embodiments, the one or more efficiency groups are attached to either end, or more preferably to each of the two ends, of the backbone of the carrier. Additionally, the length of the efficiency groups can be similar or dissimilar. In embodiments using peptoid backbones, as provided above, efficiency groups can be covalently attached at various atoms or groups of the backbone. For example, the sulfonamide-linked backbones (—SO₂NH— and —NHSO₂—) can have efficiency groups attached to the nitrogen atoms. Similarly, the hydroxyethylene (—CH(OH)CH₂—) linkage can bear efficiency groups attached to the hydroxy substituents. One of skill in the art can readily adapt the other linkage chemistries to provide efficiency groups using standard synthetic methods.

As used herein, an efficiency group is any agent that has the effect of promoting the translocation of the positively charged or hydrophobic backbone through a tissue or cell membrane and/or improving delivery of a molecule associated with the backbone to a target site. Non-limiting examples of efficiency groups include HIV-TAT or fragments thereof, the PTD of Antennapedia or a fragment thereof, or -(gly)ₙ₁-(arg)ₙ₂ (SEQ ID NO: 5) in which the subscript n1 is an integer of from 0 to about 20, more preferably 0 to about 8, still more preferably about 2 to about 5, and the subscript n2 is independently an odd integer of from about 5 to about 25, more preferably about 7 to about 17, most preferably about 7 to about 13.

In some embodiments, the HIV-TAT fragment does not contain the cysteine-rich region of the HIV-TAT molecule, in order to minimize the problems associated with disulfide aggregation. Preferably, the fragments of the HIV-TAT and Antennapedia PTDs retain the protein transduction activity of the full protein. A preferred efficiency group is, for example, -Gly₃Arg₇ (SEQ ID NO: 10). Still further preferred efficiency groups, in some embodiments, are those where the HIV-TAT fragment has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2), or (gly)ₚ-RKKRRQRRR-(gly)_{q} (SEQ ID NO; 3), wherein the subscripts p and q are each independently an integer of from 0 to about 20, or wherein p and q are each independently the integer 1. In certain preferred embodiments, p is one and q is zero or p is zero and q is one. Preferred HIV-TAT fragments are those in which the subscripts p and q are each independently integers of from 0 to 8, more preferably 0 to 5. In some embodiments, the fragment or efficiency group is attached to the backbone via either the C-terminus or the N-terminus of the fragment or amino acid sequence of the efficiency group.

In some embodiments, the efficiency groups are the Antennapedia (Antp) PTD, or a fragment thereof that retains activity. These are known in the art, for instance, from Console et al., J. Biol. Chem. 278:35109 (2003) and a non-limiting example of an Antennapedia PTD contemplated by this invention is the PTD having the amino acid sequence SGRQIKIWFQNRRMKWKKC (SEQ ID NO: 6).

In some embodiments, the efficiency groups comprise a peptide having the amino acid KLAKLAK (SEQ ID NO: 32). Other exemplary efficiency groups include any of the CPPs disclosed in US 2014/0056811 A1 to Jacob, et al., entitled “New Cell-Penetrating Peptides And Uses Thereof,” incorporated herein by reference in its entirety.

In some particularly preferred embodiments, the positively charged carrier is a positively charged peptide having the amino acid sequence RKKRRQRRR-G-(K)₁₅-G-RKKRRQRRR (SEQ ID NO: 4); or a positively charged peptide having the amino acid sequence YGRKKRRQRRR-G-(K)₁₅-G-YGRKKRRQRRR (SEQ ID NO: 7); or a positively charged peptide having the amino acid sequence RGRDDRRQRRR-G-(K)₁₅-G-RGRDDRRQRRR (SEQ ID NO: 8) for use in the compositions and methods of the invention.

Backbones and Efficiency Groups for Lipophilic Carriers

For transdermal aspects of the invention, the carrier may be a positively charged carrier having a positively charged backbone with one or more covalently attached efficiency groups, as descried above; or, alternatively, the carrier may be lipophilic. Lipophilic carriers generally comprise a hydrophobic oligomeric or polymeric backbone, to which one or more efficiency groups are covalently attached.

In particular examples where the carrier is lipophilic, the efficiency group may be selected from any of the efficiency groups described above. In addition, the efficiency group may be selected from one or more of the following: KKRPKPG (SEQ ID NO: 17); AAVLLPVLLAAP (SEQ ID NO: 18) (prion); RRRRRRRRR (SEQ ID NO: 19); RQIKWFQNRRMKWKK (SEQ ID NO: 20) (Antennapedia fragment); NPGGYCLTKWMILAAELKCFGNTAVAKCNVNHDAEFCD (SEQ ID NO: 21) (Transduction Domain 1); GIGAVLKVLTTGLPALISWIKRKRQQ (SEQ ID NO: 22) (melittin); (gly)ₚ-KKRPKPG-(gly)_{q} (SEQ ID NO: 23), wherein the subscripts p and q are each independently an integer from 0 to about 20; FLVFFFGG (SEQ ID NO: 24); and glyₙ₁ₐ-KKRPQPD-gly_{n1b} (SEQ ID NO: 25), where the subscripts n1a and n1b are each integers of from 0 to about 20.

In addition, where the carrier is a lipophilic carrier, a wide variety of synthetic or otherwise man-made efficiency groups may be used in various embodiments, including for example, KKRPKPGGGGFFFILVF (SEQ ID NO: 26), FFFILVFGGGKKRPKPG (SEQ ID NO: 27), GGGGKKRPKPG (SEQ ID NO: 28), RKKRRQRRRGGGGFFFILVF (SEQ ID NO: 29), and GGGGRKKRRQRRR (SEQ ID NO: 30), or any combination thereof and any combination with one or more other efficiency groups described herein.

In particular embodiments, the efficiency groups GGGGKKRPKPG and/or GGGGRKKRRQRRR may be bonded to a palmitoyl group, preferably bonded to n-palmitoyl, to give a lipophilic carrier. Other examples of lipophilic carriers for use in the compositions and method of the present invention include those selected from the group consisting of palmitoyl-GGRKKRRQRRR (palmitoyl-TAT, SEQ ID NO: 31); palmitoyl-glyₚ-KKRPKPG (SEQ ID NO: 11); octanoyl-glyₚ-KKRPKPG (SEQ ID NO: 12), oleyl-glyₚ-KKRPKPG (SEQ ID NO: 13), or any combination thereof, where p is an integer from 0 to about 20. Still other examples include a lipophilic carrier selected from the group consisting of FFFILVF-glyₚ-KKRPKPG (SEQ ID NO: 14), FLVFFF-glyₚ-KKRPKPG (SEQ ID NO: 15), and KKRPKPG-glyₚ-FLVFFF (SEQ ID NO: 16), or any combination thereof, where p is an integer from 0 to about 10.

Further lipophilic carriers, hydrophobic backbones, and efficiency groups (CPPs) suitable for use in the compositions and methods of the present invention include any described in US 2016/0166703 A1 to Tan et al. “Carrier Molecule Compositions and Related Methods,” each of which is incorporated herein by reference in its entirety.

Effective Amounts of the Carrier

For injectable or topical compositions of the invention, the amount of carrier is selected relative to the amount of botulinum toxin present in a composition to promote stability and/or delivery of the toxin to target sites.

Without wishing to be constrained by theory, it is believed that for carriers with positively charged backbones, the backbone forms a non-covalent electrostatic interaction with anionic surface domains of botulinum toxin to improve penetration to target tissues. It is believed that the positively charged backbone of the carrier also interacts with negatively charged extracellular structures and cell surfaces at the point of administration, such that, for example in injection strategies, these interactions restrict the botulinum toxin to the target site, reducing unwanted side effects due to spread to unintended structures. It further is believed that carriers described herein help minimize aggregation of the backbones and the botulinum toxin in therapeutic compositions, which would cause transport efficiency to decrease dramatically. In preferred embodiments, the concentration of carriers in the compositions is sufficient to enhance the delivery of the botulinum toxin to molecular targets such as, for example, motor nerve plates of one or more muscles associated with plantar fasciitis.

Furthermore, again without wishing to be bound by theory, it is believed that the penetration rate follows receptor-mediated kinetics, such that tissue penetration increases with increasing amounts of penetration-enhancing-molecules up to a saturation point, upon which the transport rate becomes constant. Thus, in preferred embodiments, the amount of carrier in a botulinum toxin-containing composition is selected to be equal, or about equal, to the amount that maximizes penetration rate right before saturation.

In some embodiments, the carrier is provided in the botulinum toxin-containing composition in an amount of about 0.001 to about 1 μg per U of the botulinum toxin component, preferably about 0.01 to about 0.5 μg per U, more preferably about 0.05 to about 0.35 μg per U or about 0.1 to about 0.3 μg per U, and most preferably about 0.234 μg per botulinum toxin unit. In some preferred embodiments, a positively charged carrier is used in an amount greater than about 10, greater than about 20, or greater than about 30 μg per 160 U of 150 kDa botulinum toxin molecule. For example, injectable compositions of the present invention may comprise about 0.16 μg/U, about 0.18 μg/U, about 0.2 μg/U, about 0.21 μg/U, about 0.22 μg/U, about 0.23 μg/U, about 0.234 μg/U, about 0.24 μg/U, about 0.25 μg/U, about 0.26 μg/U, about 0.28 mgc/U, or about 0.3 μg per U of botulinum toxin.

In some embodiments, the botulinum toxin-containing composition may contain about 10 to about 100 μg, about 20 to about 80 μg, about 30 to about 70 μg, or about 40 to about 60 μg, or about 50 μg of the carrier. In one preferred embodiment, the botulinum toxin is in a dosage amount selected from the group consisting of about 200 U to about 280 U, and the carrier is a positively charged carrier present in the composition in an amount selected from about 46 to about 66 μg, so as to provide a ratio of about 0.234 μg/U of botulinum toxin.

In one particular embodiment, the positively charged carrier is RKKRRQRRRG-(K)₁₅-GRKKRRQRRR (SEQ ID NO: 4) (also referred to herein as “RTP004”) and is present at about 12 μg per 160 U of botulinum toxin or about 45 μg per 600 U of botulinum toxin, referring to the 150 kDa toxin protein molecule. In a particular embodiment of the injectable compositions, botulinum toxin is present in an amount of about 80 U, about 120 U, or about 240 U (referring to the 150 kDa toxin protein molecule) and the RTP004 carrier is an amount of about 6 μg, about 9 μg, or about 18 μg, respectively.

Generally, mass ratio of carrier, preferably RTP004, to botulinum toxin component, preferably the 150 kDa type A toxin, is about 15,000:1 to about 60,000:1, preferably about 20,000:1 to about 55,000:1, such as about 25,000:1, about 30,000:1, about 35,000:1, about 40,000:1, about 45,000:1, or about 50,000:1. In more particular embodiments, mass ratio of carrier, preferably RTP004, to botulinum toxin component, preferably the 150 kDa type A toxin, is about 21,000:1, about 22,000:1, about 23,000:1, about 24,000:1, or about 25,000:1; in some other more particular embodiments, mass ratio of carrier, preferably RTP004, to botulinum toxin component, preferably the 150 kDa type A toxin, is about 49,000:1, about 50,000:1, about 51,000:1, about 52,000:1, or about 53,000:1. For example, per 50 U or per 100 U of toxin, the mass of the peptide carrier may be about 10 μg, about 11 μg, or about 12 μg, such as about 11.7 in some particularly preferred embodiments. In one embodiment, the molar ratio of carrier, preferably RTP004, to botulinum toxin component, preferably the 150 kDa type A toxin, is a 3:1 molar ratio of carrier:toxin.

Pharmaceutical Formulations

Pharmaceutical formulations of the compositions for use in treating or managing plantar fasciitis or a disorder related thereto, in particular for use in achieving high responder rates and/or long duration of therapeutic effect, generally are prepared by mixing the botulinum toxin component (containing the associated non-toxin proteins, reduced associated non-toxin proteins, or the 150 kD molecule alone) with a carrier described herein, and further with one or more pharmaceutically acceptable excipients or diluents suitable for injection or topical application. In their simplest form, they may contain an aqueous pharmaceutically acceptable diluent, such as buffered saline (e.g., phosphate buffered saline). The pharmaceutical formulation also may contain other ingredients typically found in injectable or topical pharmaceutical or cosmeceutical compositions, including a pharmaceutically acceptable carrier, vehicle, or medium that is compatible with the tissues to which it will be applied.

The term “pharmaceutically acceptable” describes compositions or components that are suitable for use in contacting tissues to which the compositions or components will be applied, or for use in patients in general, without undue toxicity, incompatibility, instability, allergic response, and the like. As appropriate, compositions of the invention may comprise any ingredient conventionally used in the fields under consideration.

For example, formulations for topical or injectable use may contain, as appropriate, ingredients typically used in such products, such as antimicrobials, hydration agents, tissue bulking agents or tissue fillers, preservatives, emulsifiers, natural or synthetic oils, solvents, surfactants, detergents, gelling agents, antioxidants, fillers, thickeners, powders, viscosity-controlling agents and water, and optionally including anesthetics, anti-itch actives, botanical extracts, conditioning agents, minerals, polyphenols, silicones or derivatives thereof, vitamins, and phytomedicinals.

In preferred embodiments, the botulinum toxin-containing pharmaceutical formulations do not comprise albumin or other animal protein-derived excipients. As noted above, an exogenous stabilizer (e.g., albumin) is typically added to stabilize botulinum toxin formulations. For instance, in the case of BOTOX®, 0.5 mg of human albumin per 100 U of type A botulinum toxin complex is used to stabilize the complex. In preferred embodiments, the amount of added stabilizer in botulinum toxin compositions herein is less than the amount conventionally added, owing to the ability of the carrier component to act as a stabilizer in its own right. For instance, the amount of added exogenous albumin can be any amount less than the conventional thousand-fold excess of exogenous albumin. In particularly preferred embodiments, no exogenous albumin is added as a stabilizer to the compositions of the invention, thus producing albumin-free botulinum toxin compositions. In some more particularly preferred embodiments, the formulation contains little or no other animal-derived proteins, giving an animal protein-free product.

Formulations for Injection

Injectable formulations may be in any form suitable for administration by injection and/or for storage until use in such administration. For example, injectable formulations of the compositions used to treat/manage plantar fasciitis, in accordance with this invention, may include solutions, emulsions (including microemulsions), suspensions, gels, powders, or other typical solid or liquid components used in connection with administration by injection to muscle and other target tissues in the treatment of plantar fasciitis or a related disorder.

In preferred embodiments, the compositions of the invention are present in low-viscosity, sterile formulations suitable for injection with a syringe. The compositions of the invention may be in the form of a lyophilized powder that is reconstituted for use, for example, using sterile saline or other known physiologically and pharmaceutically acceptable diluents, excipients, or vehicles, especially those known for use in injectable formulations. In certain embodiments, the lyophilized powder is reconstituted with a liquid diluent to form an injectable formulation with a viscosity of about 0.1 to about 2000 cP, more preferably about 0.2 to about 500 cP, even more preferably about 0.3 to about 50 cP, and still more preferably about 0.4 to about 2.0 cP.

In some embodiments, the injectable formulations may be in the form of controlled-release or sustained-release compositions, which comprise the botulinum toxin component and a positively charged carrier encapsulated or otherwise contained within a material such that they are released within the tissue in a controlled manner over time. For example, the composition comprising the botulinum toxin and positively charged carrier may be contained within matrixes, liposomes, vesicles, microcapsules, microspheres and the like, or within a solid particulate material, all of which is selected and/or constructed to provide release of the botulinum toxin over time. The botulinum toxin and the positively charged carrier may be encapsulated together (i.e., in the same capsule) or separately (i.e., in separate capsules).

In some embodiments, the excipient of the botulinum toxin-containing composition for injection comprises one or more additional stabilizing components. In some embodiments, compositions of the invention comprise liquid (aqueous) formulations comprising a botulinum toxin and a positively charged carrier as described herein, as well as one or more selected from the group consisting of a non-reducing sugar (such as a non-reducing disaccharide or a non-reducing trisaccharide), a non-ionic surfactant, and a physiologically compatible buffer, which is capable of maintaining a suitable pH. Suitable pH's include, for example, pH in the range of pH 4.5 to pH 7.5, or pH 4.5 to pH 6.8, or pH 4.5 to pH 6.5. It is to be understood that a suitable pH also includes the upper and lower pH values in the range, e.g., a pH of 6.5 or a pH of 7.5. Such pharmaceutical formulations are described, for example, in U.S. Pat. No. 9,340,587 to Thompson et al., entitled “Albumin-Free Botulinum Toxin Formulations;” and in US 2011/0268765 to Ruegg et al., entitled “Injectable Botulinum Toxin Formulations,” herein incorporated by reference in their entireties.

In some embodiments, the concentration of the non-reducing sugar in the liquid composition is in the range of about 10% through about 40% (w/v) and the concentration of the non-ionic surfactant is in the range of about 0.005% through about 0.5% (w/v). The liquid compositions may be dried, preferably by lyophilization, to produce stabilized solid compositions, which may thereafter be reconstituted for use, as described above. Preferably, the dried, e.g., lyophilized, solid compositions are noncrystalline and amorphous solid compositions, and may be in the form of powders.

In certain embodiments, the compositions of the invention contain a non-reducing sugar, which is preferably a disaccharide, non-limiting examples of which include trehalose, including its anhydrous and hydrated forms, or sucrose, as well as combinations thereof. In some embodiments, the hydrated form of trehalose, trehalose-dihydrate, is preferable. In other embodiments, the compositions contain a trisaccharide, a non-limiting example of which is raffinose. In general, the concentration of the non-reducing sugar, preferably a disaccharide, is in the range of 10% to 40% (w/v), preferably about 10% to about 25% (w/v), more preferably about 15% to about 20% (w/v). In some preferred embodiments, the concentration of the non-reducing sugar, preferably a disaccharide is about 10%, 11%, 12%, 13%, 14%, 15%, 16%, 17%, 18%, 19% or 20% (w/v).

In general, the compositions of the invention may include any non-ionic surfactant that has the ability to stabilize botulinum toxin and that is suitable for pharmaceutical use. In some embodiments, the non-ionic surfactant is a polysorbate, such as, by way of nonlimiting example, polysorbate 20, polysorbate 40, polysorbate 60, and polysorbate 80. In other embodiments, the non-ionic surfactant is a sorbitan ester, non-limiting examples of which include SPAN® 20, SPAN® 60, SPAN® 65, and SPAN® 80. The non-ionic surfactants Triton® X-100 or NP-40 may also be used. In addition, a combination of the different non-ionic surfactants may be used. In certain preferred embodiments, the non-ionic surfactant is a polysorbate, a poloxamer and/or a sorbitan; polysorbates and sorbitans are particularly preferred. In some embodiments, the non-ionic surfactant is present in the compositions of the invention in the range of about 0.005% to about 0.5%, about 0.01% to about 0.2%, about 0.02% to about 0.1%, or about 0.05 to about 0.08%, inclusive of the upper and lower values. In some preferred embodiments, the compositions of the invention contain a non-ionic surfactant in the amount of about 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

In general for injectable formulations herein, any physiologically compatible buffer capable of maintaining appropriate pH is suitable for use. Non-limiting examples of such buffers include salts of citric acid, acetic acid, succinic acid, tartaric acid, maleic acid, and histidine. Non-limiting examples of suitable buffer concentrations include buffer concentrations in the range of about 0.400% to about 0.600%, about 0.450% to about 0.575%, or about 0.500% to about 0.565%. The compositions of the invention may also comprise a mixture of buffer salts, non-limiting examples of which include citrate/acetate, citrate/histidine, citrate/tartrate, maleate/histidine, or succinate/histidine.

A particular composition of the invention is an albumin-free, liquid (aqueous) composition which comprises a botulinum toxin, preferably botulinum toxin of serotype A, or a botulinum toxin A having a molecular weight of 150 kDa; a positively charged carrier (e.g., peptide); a non-reducing disaccharide or a non-reducing trisaccharide, preferably a disaccharide, present in a range of 10% through 40% (w/v); a non-ionic surfactant, preferably, a polysorbate or sorbitan ester, present in the range of 0.005% through 0.5% (w/v); and a physiologically compatible buffer, such as citric acid, acetic acid, succinic acid, tartaric acid, maleic acid, or histidine, present in the range of 0.400% to 0.600%; 0.450% to 0.575%, or 0.500% to 0.565%, for maintaining the pH between 4.5. and 7.5.

In particularly preferred embodiments, the pharmaceutical formulation for injection comprises L-Histidine and/or L-Histidine hydrochloride as further stabilizing agents. In particularly preferred embodiments, the excipient comprises trehalose dihydrate, polysorbate 20, L-histidine and L-histidine hydrochloride.

Formulations for Topical Administration

In alternative embodiments, the compositions of the invention are formulated for application to the skin or epithelium of individuals in need to treatment for plantar fasciitis or a disorder related thereto. Topical formulations may be in any form suitable for topical administration and/or for storage until use in such administration.

For example, topical formulations of the compositions used to treat/manage plantar fasciitis, in accordance with this invention, may include solutions, emulsions (including microemulsions), suspensions, creams, lotions, gels, powders, or other typical solid or liquid compositions used for application to skin of the foot region, in particular, to areas of the ankle, heel, and plantar arch.

The topical formulations may contain, in addition to the carrier and botulinum toxin component, other ingredients typically used in such products, such as antimicrobials, moisturizers and hydration agents, penetration agents, preservatives, emulsifiers, natural or synthetic oils, solvents, surfactants, detergents, emollients, antioxidants, fragrances, fillers, thickeners, waxes, odor absorbers, dyestuffs, coloring agents, powders and optionally including anesthetics, anti-itch additives, botanical extracts, conditioning agents, humectants, minerals, polyphenols, silicones or derivatives thereof, sunblocks, vitamins, and phytomedicinals.

In some preferred embodiments, the topical formulations will include gelling agents and/or viscosity-modifying agents. These agents are generally added to increase the viscosity of the formulation, so as to make topical application of the composition easier and more accurate. Additionally, these agents help to prevent the aqueous botulinum toxin/carrier solution from drying out, which tends to cause a decrease in the activity of the botulinum toxin. Particularly preferred agents are those that are uncharged and/or that do not interfere with the botulinum toxin activity or the efficiency of the toxin-carrier complexes in terms of crossing the skin.

In particular embodiments, the gelling agents are certain cellulose-based gelling agents, such as hydroxypropylcellulose (HPC). In some embodiments, topical formulations will have about 2 to about 4% HPC. Alternatively, the viscosity of the topical formulation may be altered by adding polyethylene glycol (PEG) or poloxamer. In other embodiments, the botulinum toxin/carrier solution is combined with pre-mixed viscous agents, such as Cetaphil® moisturizer.

The viscosity modifier optionally may be a surfactant. The surfactant may be selected from anionic surfactants, cationic surfactants, zwitterionic surfactants, or non-ionic surfactants. In certain embodiments, one or more non-ionic surfactants serve as the viscosity modifier. The non-ionic surfactant can be any commercially available non-ionic surfactant, such as, for example, polyoxyethylene glycol alkyl ethers, polyoxypropylene glycol alkyl ethers, glucoside alkyl ethers, polyoxyethylene glycol octylphenol ethers, polyoxyethylene glycol alkylphenol ethers, glycerol alkyl esters, polyoxyethylene glycol sorbitan alkyl esters, sorbitan alkyl esters, dodecyldimethylamine oxide, block copolymers of polyethylene glycol and polypropylene glycol (polyoxamers), and combinations thereof.

In certain embodiments, the non-ionic surfactant is a polysorbate, non-limiting examples of which include polysorbate 20, polysorbate 40, polysorbate 60, and polysorbate 80. In other embodiments, the non-ionic surfactant is a sorbitan ester, non-limiting examples of which include Span 20, Span 60, Span 65, and Span 80. The invention also contemplates using Triton X-100, trileucine, or NP-40 as the non-ionic surfactants. In addition, the combinations of different non-ionic surfactants are contemplated. In certain preferred embodiments, the non-ionic surfactant is selected from the group consisting of polysorbates, poloxamers, and sorbitans, with polysorbates and sorbitans being particularly preferred.

In a particular embodiment, the viscosity modifier is a poloxamer. Such poloxamers may be linear or branched, and include tri-blocks or tetra-blocks copolymers, as well as poloxamines such as Tetronic and Pluronic. The poloxamer may be chosen from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, and combinations thereof. In certain preferred embodiments, the poloxamer that is chosen has a tendency to form a gel with increasing temperature. Non-limiting examples of such poloxamers include poloxamer 188 and poloxamer 407.

As the skilled artisan will appreciate, the amount of viscosity modifying agent that is present in the topical botulinum toxin formulations of the invention will depend on the identity of the viscosity modifying agent, as well as the desired viscosity of the topical formulation. With this in mind, suitable concentrations for a viscosity modifying agent in the formulations described herein may range from about 5% to about 70% (wt/wt), such as, for example, between about 5% to about 60%, between about 10% to about 50%, or between about 15% to about 40%. Optionally, the viscosity modifier is present in the compositions in a concentration between about 15% to about 20%, such as, for example, about 16%, about 17%, about 18%, about 19% or about 20%. Optionally, the viscosity modifying agent is present in a concentration of about 15.0%, 15.5%, 16.0%, 16.5%, 17.0%, 17.5%, 18.0%, 18.5%, 19.0%, 19.5%, or 20% and is selected from the group consisting of poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, and combinations thereof.

For example, in certain preferred embodiments when the poloxamer is poloxamer 407, the amount of poloxamer present in the formulations ranges from 15-25%, 15.5-24.5%, 16-23%, 16.5-22.5%, 17-22% 17.5%-21.5%, or 18%-21%. The amount of poloxamer 407 may be about 15.5%, 16.0%, 16.5%, 17.0%, 17.5%, 18.0%, 18.5%, 19.0%, 19.5%, 20%, 20.5%, 21% 21.5% or 22%. Alternatively, the chosen poloxamer is poloxamer 188, which may be present in the formulations of the invention at a concentration of about 15.5%, 16.0%, 16.5%, 17.0%, or 17.5%. In some embodiments, more than one type of poloxamer is used to modify the viscosity of the topical formulation. For instance, in some particularly preferred embodiments, both poloxamer 188 and poloxamer 407 are added to the topical formulation to modify viscosity. Other formulations for topical application of the composition described herein can be found in US 2014/0120077, to Ruegg et al., entitled “Compositions and Methods for Safe Treatment of Rhinitis,” which is incorporated herein in its entirety.

In some embodiments regarding topical administration, the composition is formulated for delivery via an emulsion or a liposomal preparation. Emulsion preparations may be used with positively charged carriers, where the carrier (along with the non-covalently associated botulinum toxin component) adheres or associates to a plurality of micelles in the emulsion. The emulsion may then be mixed with additional components in one or more liquid/solid phases to form a final composition formulated for administration to the skin of an individual having plantar fasciitis, such as the skin overlying one or more muscles or fascia associated with plantar fasciitis pain, such as the skin on the heel, ankle, and/or plantar arch.

Liposomal preparations typically are used with lipophilic carriers, that is, in compositions comprising a botulinum toxin component and a lipophilic carrier. Generally, the botulinum toxin/carrier complex is packaged into a liposome. In some embodiments, the liposomes are obtained according to technologies used by Encapsula NanoSciences of Brentwood, Tenn.; Lippomix, Inc. of Novato, Calif.; Azaya Therapeutics Incorporated of San Antonio, Tex.; Oakwood Laboratories, L.L.C. of Oakwood Village, Ohio; Tergus Pharma of Durham, N.C., and/or as otherwise known in the art. A plurality of liposomes may be prepared and mixed with additional components in one or more liquid/solid phases to form a final composition formulated for topical administration to the skin of an individual having plantar fasciitis, such as the skin overlying one or more muscles or fascia associated with plantar fasciitis pain, such as the skin on the heel, ankle, and/or plantar arch.

Additional approaches for preparing topical formulations of botulinum toxin-containing compositions, for use in the present invention, can be found in US 2016/0166703 A1 to Tan et al. “Carrier Molecule Compositions and Related Methods,” which is incorporated herein by reference in its entirety.

Use of the Injectable and Topical Formulations

In another aspect of the invention, the pharmaceutical formulations described herein are used in the treatment and management of plantar fasciitis or a disorder related thereto, preferably to achieve an extended duration therapeutic effect, for example, in reducing pain associated with plantar fasciitis (“plantar fasciitis pain”). The pharmaceutical formulation generally is administered to an individual in need thereof to provide a therapeutically or cosmetically effective amount of botulinum toxin. The term “in need,” in reference to subjects or patients, is meant to include subjects or patients suffering from at least one symptom typically associated with plantar fasciitis. A disorder related to plantar fasciitis means a disorder sharing one or more symptoms typical of plantar fasciitis. The term “therapeutically effective amount” as used herein refers to a dose of botulinum toxin needed to produce at least one therapeutic effect with regard to plantar fasciitis or a related disorder, such as the effect of reducing, attenuating, or eliminating one or more symptoms of plantar fasciitis.

Typical symptoms of plantar fasciitis include, e.g., foot pain with weight bearing (classically more pain is experienced in the morning than in the afternoon) and tenderness associated with pressure on the plantar arch, as well as tenderness of the plantar fascia, tenderness at the calcaneus, relative equinus contracture of the ankle (with the hind foot stabilized), reproduction of pain with palpation of the proximal insertion of the plantar fascia, and positive Tinel's sign for the posterior tibial, medial plantar, and/or lateral plantar nerves. In particular embodiments, the therapeutic effect comprises reducing, attenuating, or eliminating one or more of these symptoms, in particular reducing pain associated with inflammation of the plantar fascia, including a reduction in the severity of the pain and/or a reduction in the frequency of the pain. Generally, a therapeutically effective amount of botulinum toxin in the treatment or management of plantar fasciitis is an amount sufficient to produce a desired muscular paralysis of one or more muscles associated with plantar fasciitis. The therapeutically effective amount also is implicitly a safe amount, i.e., one that is low enough to avoid serious side effects or to avoid side effects that are not outweighed by the beneficial effects of the treatment.

In particular embodiments, the therapeutic effect is the paralysis or relaxation of one or more muscles selected from the group consisting of plantar spur (periosteum), a short flexor, quadratus plantae, triceps surae (gastrocnemium and soleus), flexor digitorum brevis, and flexor hallucis longus. Muscle relaxation or paralysis may alleviate unwanted contractions and/or prevent or alleviate spasms of the targeted muscle. In some embodiment, the therapeutic effect is improvement in ankle function, heel pain, and/or shoe comfort. In preferred embodiments, the therapeutic effect includes ameliorating symptoms of plantar fasciitis so as to improve function of the affected foot (or feet) and/or to improve health-related quality of life for the plantar fasciitis patient.

Plantar fasciitis can be classified by assessment using the Foot Posture Index-6 (FPI-6) scale. In one study, the mean FPI-6 score for chronic plantar heel pain was 2.4±3.3, versus 1.1±2.3 for controls (Irving et al., BMC Musculoskelet Disord. 2007 8(41)). Diagnosis may be made based on history and physical examination, considering the following: plantar medial heel pain (worse upon initial steps after a period of inactivity and/or following prolonged weight bearing); heel pain precipitated by a recent increase in weight bearing; pain with palpation of the proximal insertion of the plantar fascia; positive windlass test; negative tarsal tunnel tests; limited active and passive talocrural joint dorsiflection range of motion; abnormal FPI score; and/or high body mass index in nonathletic individuals. Spondyloarthritis, fat-pad atrophy, and proximal plantar fibroma may also be assessed. Diagnostic ultrasound also may be used to assess plantar fascia thickness, as increased plantar fascia thickness is associated with the disorder.

Although the etiology of plantar fasciitis is not understood completely, foot pain occurs with weight bearing and is accompanied by: 1) functional shortening of the short plantar muscles and plantar fascia; 2) tenderness at the insertion of the plantar fascia on the calcaneus (with or without a spur); and 3) shortening/tightening of the gastrocnemius/soleus (equinus). In addition, the plantar nerves may become entrapped or inflamed. Heel pain associated with plantar fasciitis is produced by nociceptive foci within degenerative plantar fascia adjacent to and distal to its origin. Shortening of the plantar fascia occurs during sleep and inactivity; then the forces brought about by standing stress the degenerative fascia and produce pain. It is unknown whether the adaptations within the gastrocnemius-soleus complex (triceps surae) producing shortening of muscle fibers is primary or secondary; however, reestablishment of the length of the Achilles tendon decreases forces within the plantar fascia.

Without being bound by theory, it is believed that, following injection of a composition of the invention into the plantar muscle or plantar fascia, the triceps surae will be rendered partially dysfunctional by the toxin, thus decreasing forces and/or equinus deformity at the ankle resulting in elongation of the plantar structures. Attendant decreased pain and inflammation then may minimize nerve irritation. It is hypothesized that biomodulation with a composition of the invention, administered to the plantar structures (fascia, fascial origin, and short flexors), decreases tension in the plantar structures, blocking nociceptive discharges within degenerative fascia and the facial origin (the spur), and thus diminishing forces that contribute to pain and allowing healing to take place. Again without being bound by theory, it is believed that injection of a composition of the invention into the triceps surae (gastrocnemius and soleus) may decrease the equinus (plantar) moment, thereby diminishing stress along the plantar fascia plane, further promoting healing and relief of plantar fasciitis pain. Optimal outcomes, in certain embodiments, are achieved when the plantar muscles are relaxed, the pain fibers in the plantar fascia and/or calcaneal spur are decreased, and/or the gastrocnemius/soleus complex is relaxed with decreased relative equinus.

Improvement in plantar fasciitis can be measured by one or more standardized approaches. Measures include, for example, improvement in the Numeric Pain Rating Scale (NPRS), Foot Function Index (FFI), Patient Global Impression of Change (PGIC), Clinician Global Impression of Change (CGIC), Treatment Satisfaction Questionnaire (TSQ), American Orthopedic Foot and Ankle Score (AOFAS); improvement in Foot and Ankle Disability Index (FADI); improvement in Patient Reported Outcome Measurement Information Study (PROMIS®) http://www.nihpromis.org/; a reduction in visual analog pain score (visual analog scale or “VAS”) for the foot; and/or the plantar fasciitis pain and disability scale (PFPS). Other measures of improvement of plantar fasciitis include validated self-report measures, such as Foot and Ankle Ability Measure (FAAM), Foot Health Status Questionnaire (FHSQ), and Lower Extremity Function Scale (LEFS, in particular, a computer-adaptive version).

Regarding AOFAS, the American Academy of Orthopaedic Surgeons has developed several approaches to collect patient-based data in assessing the effectiveness of treatments and to study the clinical outcomes of the treatments. The AOFAS foot and ankle questionnaire was designed for use in patients 18 years old and older. This questionnaire documents patient assessments of foot and ankle conditions and improvements resulting from treatments. Disability indices for the lower limb core, global foot, and ankle function, and shoe comfort can be evaluated using this approach.

PROMIS® represents a system of highly reliable, precise measures of patient-reported health status for physical, mental, and social well-being (http://www.nihpromis.org/) PROMIS tools measure what patients are able to do and how they feel by asking questions. PROMIS measures can be used across a wide variety of chronic diseases and conditions in clinical studies of the effectiveness of treatment.

FADI contains 34 questions divided into two subscales: the Foot and Ankle Disability Index and the Foot Ankle Disability Index Sport (Martin, et al. Development of the Foot and Ankle Disability Index (FADI) [abstract]. J Orthop Sports Phys Ther. 1999). Each of the questions is scored using a five-point Likert Scale from 0 (unable to do) to 4 (no difficulty at all). The FADI was designed to collect information from patients regarding their functional limitations related to their feet and ankles. The FADI Sports module is a population specific subscale designed for athletes.

The Visual Analog Scale (VAS) for pain is a continuous scale self-completed by the respondent comprised of a horizontal (HVAS) or vertical (VVAS) line, usually 10 centimeters (100 mm) in length, anchored by 2 verbal descriptors, one for each symptom extreme. Instructions, time period for reporting, and verbal descriptor anchors have varied widely in the literature depending on intended use of the scale. For pain intensity, the scale is most commonly anchored by “no pain” (score of 0) and “pain as bad as it could be” or “worst imaginable pain” (score of 100 [100-mm scale]). The respondent is asked to place a line perpendicular to the VAS line at the point that represents their pain intensity (http://onlinelibrary.wiley.com/doi/10.1002/acr.20543/full).

The PFPS is a survey that includes a series of key questions that relate to symptoms and control questions for plantar fasciitis. The PFPS also includes the VAS for pain and questions to measure the effect the pain has on their activities of daily living. This survey was designed to create a more descriptive, exclusive analysis for plantar fasciitis and has been shown to effectively discriminate pain that is unique to plantar fasciitis versus heel pain caused by other foot pathologies.

(https://faoj.files.wordpress.com/2009/05/pain_scale_for_plantar_fasciits.pdf).

Additional measures are as follows:

The Numeric Pain Rating Scale (NPRS for Pain): (Ferreira et al., 2011, “Validity of four pain intensity rating scales”; retrieved from https://www.ncbi.nlm.nih.gov/pubmed/21856077), McCaffery et al., 1989, Pain: Clinical manual for nursing practice, Mosby St. Louis, Mo.; Jensen et al., 2015, “Assessment of Pain Intensity in Clinical Trials: Individual Ratings vs Composite Scores”, Pain Medicine, 16: 141-148, Wiley Periodicals, Inc.; and Dworkin et al., 2005, Pain, 113 (2005):9-19). The NPRS is a segmented numeric version of the visual analog scale (VAS) in which a respondent selects a whole number (0-10 integers) that best reflects the intensity of his/her pain. The common format is a horizontal bar or line. Similar to the VAS, the NPRS is anchored by terms describing pain severity extremes.

Foot Function Index (FFI): The FFI (Budiman-Mak et al., 1991, “The Foot Function Index: a measure of foot pain and disability,” Journal of clinical epidemiology, 44(6):561-70) was developed to measure the impact of foot pathology on function in terms of pain, disability and activity restriction and is a self-administered index consisting of 23 items divided into 3 sub-scales. Both total and sub-scale scores are produced. The FFI can be applied to the effect of plantar fasciitis on foot function.

Foot and Ankle Ability Measure (FAAM): The FAAM was developed to comprehensively assess physical performance among individuals with leg and ankle musculoskeletal disorders (Martin et al, 1999, “Development of the Foot and Ankle Disability Index (FADI) [abstract],” J Orthop Sports Phys Ther, (29):A32-A3); and Martin et al, 2005, “Evidence of Validity for the Foot and Ankle Ability Measure,” Foot and Ankle International. 26(11):968-983. The FAAM can be applied to performance as relates to plantar fasciitis.

Clinician Global Impression of Change (CGIC): The CGIC is a questionnaire that captures the clinician's overall impression of the subject's response to study treatment. The clinician's selected response maps to a 7-point scale: −3 (very much worse), 0 (about the same), to +3 (very much better). (Guy, 1976 Editor. ECDEU Assessment Manual for Psychopharmacology. Rockville, Md., US Department of Health, Education, and Welfare Public Health Service Alcohol, Drug Abuse, and Mental Health Administration).

Patient Global Impression of Change (PGIC): The PGIC is a questionnaire that captures the patient's overall impression scale: −3 (very much worse), 0 (about the same), to +3 (very much better). (Farrar et al, 2001, “Clinical importance of changes in chronic pain intensity measured on an 11-point numerical pain rating scale,” Pain, 94:149-158).

Treatment Satisfaction Questionnaire (TSQ): The TSQ is self-administered instrument that measures the patient's overall satisfaction with his or her study treatment. (Revicki, 2004 “Patient assessment of treatment satisfaction: methods and practical issues,” Gut, 53).

In preferred embodiments, a combination of methods is used for evaluating reduction in pain experienced following treatment for plantar fasciitis, such as a combination comprising two or more of the methods described above. In a particularly preferred embodiment, the validated visual analog scale (VAS) is used for measuring reduction of pain in an affected foot as a primary endpoint; and an improvement in the American Orthopaedic Foot and Ankle Score (AOFAS) is used as a secondary endpoint. This combination can provide clinically meaningful measurements of the ability of the present compositions and methods to reduce the pain associated with plantar fasciitis and effectively treat the condition.

In still more preferred embodiments, the validated visual analog scale (VAS) is used for measuring reduction of pain in an affected foot as a primary endpoint; and an improvement in one or more of the following is used as a secondary endpoint: validated visual analog scale (VAS); American Orthopaedic Foot and Ankle Score (AOFAS); Foot and Ankle Disability Index (FADI); Patient Reported Outcome Measurement Information Study (PROMIS®); and/or the plantar fasciitis pain and disability scale (PFPS). The affected foot also may be X-rayed for signs of improvement.

In another particularly preferred embodiment for assessing efficacy of treatment, the NPRS is used for measuring reduction of pain in an affected foot as a primary efficacy endpoint; the FFI and/or NPRS are used as secondary efficacy endpoints; and/or the NPRS, PPT, CGIC, PGIC, and/or FAAM are used for exploratory efficacy endpoints. Pain measurement may be recorded based on the first steps out of bed in the morning, e.g., within the first about 10 minutes, about 15 minutes, about 20 minutes, about 30 minutes, about 45 minutes, or about 60 minutes, preferably within the first 15 minutes, after getting out of bed in the morning.

In some embodiments, methods of the invention produce an effect of reduction in severity of plantar fasciitis (according to any of the scales described above), preferably a reduction in moderate to severe plantar fasciitis pain. A reduction in severity may be a 1 point, 2 point, or 3 point improvement, or more, in one or more assessment scales described herein. Methods of treatment achieve surprisingly long duration and high responder rates. For example, in particular embodiments, the interval before administering a second or subsequent treatment dose of the composition is greater than or equal to about 20 weeks, about 22 weeks, about 24 weeks, about 26 weeks, about 28 weeks, about 30 weeks, about 32 weeks, about 34 weeks, about 36 weeks, about 38 weeks, about 40 weeks, or greater than or equal to about 42 weeks, following the initial treatment dose or following subsequent treatment doses. In preferred embodiments, the effect endures for at least about 4 weeks in over 55% over 56%, over 58%, over 60%, over 62%, over 65%, over 66%, over 68%, over 70%, over 72%, over 73%, or over 75% of individuals each administered the pharmaceutical composition. In more preferred embodiments, the effect endures for at least about 16 weeks in over 35%, over 36%, over 38%, over 40%, over 43%, over 45%, over 47%, over 50%, over 53%, over 55%, over 57%, over 60%, over 63%, over 65%, over 68%, more preferably over 70%, over 73%, or over 75%, of individuals each administered the pharmaceutical composition. In even more preferred embodiments, the effect endures for at least about 24 weeks in over 15%, over 16%, over 18%, over 20%, over 22%, over 23%, over 25%, over 27%, or over 30%, of individuals each administered the pharmaceutical composition.

In certain embodiments, treatment results in about 10%, about 20%, about 30%, about 40%, about 50%, about 60%, about 70%, or about 80% reduction in pain, preferably as measured by the VAS for pain, or the NPRS for pain, and this pain reduction lasts through weeks one, two, three, four, five, six, seven, eight, nine, ten, eleven, twelve, thirteen, fourteen, fifteen, or sixteen, following treatment. In a particular embodiment, plantar fasciitis pain is reduced by 50%, or more, through week eight following a single injection treatment, more preferably following a single injection. In some particular embodiments, a single treatment with RT002 reduces patient-reported VAS for pain, or NPRS for pain, by at least about 30%, at least about 40%, at least about 50%, at least about 60%, or at least about 70%, though least about 8 weeks or later following the treatment. In some preferred embodiments, a single treatment with RT002 reduces pain by at least about 50%, at least about 51%, at least about 52%, at least about 53%, at least about 54%, at least about 55%, at least about 56%, at least about 58%, or at least about 60%, through about 6 weeks, about 7 weeks, about 8 weeks, about 9 week, about 10 weeks, or about 12 weeks or longer, following the treatment, as assessed by patient-reported VAS for pain or NPRS for pain. In a particularly preferred embodiment, a single treatment with RT002 reduces patient-reported VAS for pain by at least about 50% at 8 weeks following the treatment. See Example 1.

Use of a single injection, such as a single injection through one site, provides the additional advantage of reduced discomfort to the subject, compared to treatments using multiple injection sites, as well as convenience and speed of the treatment, and reduced risk of spread of the toxin and reduced risk of damage to surrounding tissues following administration. In certain embodiments the single injection may be administered in one site, but may also be divided into two or more separate sites.

In some embodiments, the compositions and methods of the present invention are used in combination with a standard and/or non-standard therapy for plantar fasciitis, including conservative therapies for less severe cases and more aggressive approaches attempted in more severe cases. Conservative therapies for treating plantar fasciitis include leg and foot stretching exercises, to stretch plantar fascia and gastrocnemius/soleus complex muscles, as well as manual therapy, nonsteroidal anti-inflammatory drugs, corrective shoe inserts, heel pads, taping, splinting, and/or night splints. More aggressive approaches for treating plantar fasciitis include steroid injections, such as cortical injections, platelet rich plasma injections, traditional botulinum toxin injections, phonophoresis, ultrasound, electrotherapy, extracorporeal shock wave therapy, and/or surgery.

As used herein, the term “in combination” or “in further combination” or “further in combination” refers to the use of an additional therapeutic approaches as well as a composition or method of the invention. The use of the term “in combination” does not restrict the order in which approaches are used. In some embodiments, injection of a composition of the invention into a muscles or fascia associated with plantar fasciitis is combined with one or more of the following: leg/foot stretching exercises, e.g., stretching plantar fascia and gastrocnemius/soleus complex; strengthening exercises, movement training, dry needling, manual therapy, such as joint and soft tissue mobilization, taping, splinting, night splints, electrotherapy, phonophoresis (e.g., with ketoprofen gel), iontophoresis, low-level laser therapy, ultrasound, extracorporeal shockwave therapy, shoe inserts (e.g., orthoses, particularly for support of the medial longitudinal arch), heel pads (silicone heel pads, felt pads, rubber heel cups, or custom-made polypropylene orthotic devices (Pfeffer, et al. “Comparison of custom and prefabricated orthoses in the initial treatment of proximal plantar fasciitis,”. Foot Ankle Int. 1999 April; 20(4):214-21)), “rocker-bottom” shoe constructs; shoe rotation during the work week; steroid injections (e.g., corticosteroid injections), traditional botulinum toxin injections (Seyler, 2008), extracorporeal shock wave therapy, platelet rich plasma injections, and surgery (Martin, et al. “Heel pain-plantar fasciitis: revision” 2014, J Orthop Sports Phys Ther. 2014 November; 44(11):A1-33), e.g., plantar fasciotomy; gastrocnemius recession, such as proximal medial astrocnemius release (Monteagudo, et al. “Chronic plantar fasciitis: plantar fasciotomy versus gastrocnemius recession,” Int Orthop. 2013 September; 37(9):1845-50); and/or partial plantar fasciectomy with neurolysis of the nerve to the abductor digiti quinti muscle (Sammarco, et al. “Surgical treatment of recalcitrant plantar fasciitis,” Foot Ankle Int. 1996 September; 17(9):520-26), including surgery to release the plantar fascia at the midsection or proximally, proximal release of the plantar fascia and ostectomy of the calcaneal spur, endoscopic plantar fasciectom, and recession of the gastrocnemius and/or soleus (Davies et al. “Plantar fasciitis: how successful is surgical intervention?” Foot Ankle Int. 1999 December; 20(12):803-7; League “Current concepts review: plantar fasciitis,” Foot Ankle Int. 2008 March; 29(3):358-66; Monteagudo et al. “Chronic plantar fasciitis: plantar fasciotomy versus gastrocnemius recession,” Int Orthop. 2013 September; 37(9):1845-50; Neufeld et al. “Plantar fasciitis: evaluation and treatment,” J Am Acad Orthop Surg. 2008 June; 16(6):338-46; and Sammarco et al. “Surgical treatment of recalcitrant plantar fasciitis,” Foot Ankle Int. 1996 September; 17(9):520-26).

As noted above, it is believed that, following injection of a composition of the invention into the plantar muscle or plantar fascia, the triceps surae will be rendered partially dysfunctional by the toxin, thus decreasing forces and/or equinus deformity at the ankle resulting in elongation of the plantar structures. Attendant decreased pain and inflammation then may minimize nerve irritation. It is hypothesized that biomodulation with a composition of the invention, administered to the plantar structures (fascia, fascial origin, and short flexors), decreases tension in the plantar structures, blocking nociceptive discharges within degenerative fascia and the facial origin (the spur), and thus diminishing forces that contribute to pain and allowing healing to take place. Accordingly, in a particularly preferred embodiment, use of a stretching/splinting, in combination with treatment according to the present invention, will maximize the toxin effects, for example, by relaxing the plantar muscles, addressing pain fibers in the plantar fascia and calcaneal spur, and/or relaxing the gastrocnemius/soleus complex, with decreased relative equinus.

Dosage and Administration

Methods and compositions described herein deliver the botulinum toxin component in a dose or amount effective to improve at least one symptom of plantar fasciitis or a related disorder, as discussed above, preferably for an extended duration. Generally, therapeutically effective amounts are provided as doses in botulinum toxin units contained in the pharmaceutical formulations for administration by injection or transdermal delivery, in accordance with the present invention.

In certain embodiments using injectable formulations, the botulinum toxin is administered to provide about 1 U to about 1,000 U, preferably from about 100 U to about 500 U, more preferably from about 150 U to about 350, or from about 200 U to about 300 U; or more specifically, from about 220 U to about 280 U, from about 220 U to about 260 U, or about 240 U per injection treatment. An “injection treatment” refers to a single treatment that may comprise one or more injections to the patient, e.g., all within a single patient visit, such as a series of injections administered within seconds or minutes of each other; and/or administered in the same general area of the patient's body (e.g., the foot and ankle muscles) through one or more injection sites in relative close proximity (e.g., about 1 cm, about 2 cm, about 3 cm, about 4 cm, or about 5 cm apart).

In some preferred embodiments, the botulinum toxin-containing compositions of the invention are administered to a subject in need thereof by injection, so as to provide a dose greater than about 50 U, for example, at least about 75 U, at least about 100 U, at least about 150 U, at least about 200 U, least about 220 U, least about 240 U, at least about 250 U, or at least about 260 U; or about 200 U, about 220 U, about 230 U, about 240 U, about 250 U, about 260 U, about 280 U, about 300 U, about 320 U, about 340 U, about 360 U, about 380 U, about 400 U, about 500 U, about 600 U, about 700 U, or about 800 U; or 200 U, 220 U, 230 U, 240 U, 250 U, 260 U, 280 U, 300 U, 320 U, 340 U, 360 U, 380 U, or 400 U of the botulinum toxin, preferably botulinum toxin of serotype A, more preferably the 150 kDa molecule of serotype A botulinum toxin. Amounts or doses between the foregoing amounts or doses are also contemplated, for example, 225 U or at least 225 U; 235 U or at least 235 U; 245 U or at least 245 U, and the like. In particularly preferred embodiments, the botulinum toxin is in a dosage amount selected from the group consisting of about 210 U, about 220 U, about 230 U, about 240 U, about 250 U, about 260 U, and about 270 U, more preferably botulinum toxin of serotype A, most preferably the 150 kDa molecule of serotype A botulinum toxin. Generally, an amount of about 100 pg/kg of the 150 kDa molecule of botulinum toxin A will correspond to about 16 U/kg, in liquid injectable formulations of the present invention.

In certain embodiments, the botulinum toxin is administered from about 1 U to about 1,000 U, preferably from about 20 U to about 200 U, more preferably from about 40 U to about 180 U; or more specifically, from about 50 U to about 160 U, from about 60 U to about 150 U, from about 70 U to about 130 U, or about 80 U to about 120 U per injection treatment. In preferred embodiments, the botulinum toxin is in a dosage amount selected from the group consisting of about 50 U, about 60 U, about 70 U, about 80 U, about 90 U, about 100 U, about 110 U, about 120 U, about 130 U, about 140 U, about 150 U, and about 160 U. In some such embodiments, the injection treatment is a single injection.

In some preferred embodiments, the botulinum toxin-containing compositions of the invention are administered to a subject in need thereof by injection, so as to provide a dose greater than about 20 U, for example, at least about 40 U, at least about 60 U, at least about 80 U, at least about 100 U, least about 120 U, least about 140 U, at least about 160 U, or at least about 180 U; or about 60 U, about 70 U, about 75 U, about 80 U, about 85 U, about 90 U, about 100 U, about 110 U, about 115 U, about 120 U, about 125 U, about 130 U, about 140 U, about 150 U, about 160 U, about 170 U, or about 180 U; or 60 U, 65 U, 70 U, 75 U, 80 U, 85 U, 90 U, 95 U, 100 U, 105 U, 110 U, 115 U, 120 U, 125 U, 130 U, 135 U, or 140 U of the botulinum toxin, preferably botulinum toxin of serotype A, more preferably the 150 kDa molecule of serotype A botulinum toxin. Amounts or doses between the foregoing amounts or doses are also contemplated. In particularly preferred embodiments, the botulinum toxin is in a dosage amount selected from the group consisting of about 50 U, about 60 U, about 70 U, about 80 U, about 90 U, about 100 U, about 110 U, about 120 U, about 130 U, about 140 U, and about 150 U, more preferably botulinum toxin of serotype A, most preferably the 150 kDa molecule of serotype A botulinum toxin. Generally, an amount of about 100 pg/kg of the 150 kDa molecule of botulinum toxin A will correspond to about 16 U/kg, in liquid injectable formulations of the present invention.

The pharmaceutical formulations of the invention may contain a therapeutically effective amount of the botulinum toxin for application as a single-dose treatment, such as a single injection or a single topical application. Alternatively, the pharmaceutical formulations may be more concentrated, e.g., for dilution at the place of administration, or may contain therapeutically effective amounts of the botulinum toxin for use in multiple applications, such as use in a specified number of sequential applications over a course of treatment or over a period of time. Local delivery of the botulinum toxin, as described herein, may afford dosage reductions, reduce toxicity, and allow more precise dosage optimization for desired effects relative to conventional botulinum toxin formulations.

In preferred embodiments, the dose (e.g., in units and the volume) is selected to optimize delivery of the toxin to target receptor/neurotransmitter containing muscle or fascial/periosteal nociceptors. Optimization may be based, for example, on dose dilution distribution principles (see, e.g., U.S. Pat. Nos. 8,632,768 and 8,506,970).

Generally, the botulinum toxin-containing pharmaceutical formulation is administered to a patient in need thereof by injection into one or more of the muscles or fascia associated with plantar fasciitis; and/or is topically applied to skin overlying one or more of these muscles or fascia of a patient in need thereof. Administration may comprise intramuscular or non-intramuscular injection (typically using a syringe) into, or near to, one or more of muscles selected from the group consisting of plantar spur (periosteum), a short flexor, quadratus plantae, triceps surae (gastrocnemium and soleus), flexor digitorum brevis, and flexor hallucis longus; or injection into or near to the plantar fascia, such as at the at the medial calcaneal tubercle; and/or administration may comprise topical application to skin overlying one or more of the above-recited structures or in the general area of the heel, ankle, and/or plantar arch. Administration “near to” or “at” a structure means administration close enough to the structure to allow the botulinum toxin component to readily diffuse to the structure, taking into consideration the reduced diffusion of the botulinum toxin compositions disclosed herein. For example, administration near to the plantar fasciitis means administration within about 0.05 mm, about 0.1 mm, about 0.5 mm, about 1 mm, about 5 mm, about 10 mm, about 15 mm, or about 20 mm of the structure. In certain embodiments, ultrasound or other visualization techniques may be used to guide placement of the injection, or injection fractions.

In more particular embodiments, specific dose amounts are injected into specific structures; for example, in one embodiment, a dose of about 50 U to about 300 U, about 100 U to about 200 U, or about 160 U of botulinum toxin is injected into triceps sura; and a dose of about 10 U to about 150 U, about 50 U to about 100 U, or about 80 U of botulinum toxin is injected into at least one structure selected from the group consisting of plantar fascia, plantar spur (periosteum), a short flexor, and quadratus plantae. In particular embodiments, administration may comprise injection into one or more of muscles or fascia selected from the group consisting of plantar fascia (preferably the point of maximum tenderness in the plantar fascia), plantar spur (periosteum, preferably the periosteum over the plantar insertion), a short flexor, quadratus plantae, and triceps surae (gastrocnemium and soleus). In other embodiments, administration may comprise topical application to skin overlying one or more of the above-recited muscles and fascia. In more particular embodiments, specific dose amounts are injected into specific muscles or fascia; for example, in one embodiment, a dose of about 50 U to about 300 U, about 100 U to about 200 U, or about 160 U of the botulinum toxin component is injected into triceps sura; and a dose of about 10 U to about 150 U, about 50 U to about 100 U, or about 80 U of the botulinum toxin component is injected into at least one muscle or fascia selected from the group consisting of plantar fascia, plantar spur (periosteum), a short flexor, and quadratus plantae.

In a particular example, a dose of about 160 U of the botulinum toxin component is injected into triceps sura, at about 2 cm intervals, e.g., at about 1, 2, 3, 4, 5, 6, 7, 8, 9, or 10 skin injection sites, preferably about 2-9, about 3-8, or about 4-7 sties, depending on the size of the subject's muscles; and/or a dose of about 80 U of the botulinum toxin component is injected into at least one muscle or fascia selected from the group consisting of plantar fascia (preferably a point of tenderness, more preferably a point of maximum tenderness), plantar spur (periosteum, preferably in the region of the plantar insertion, more preferably over the plantar insertion), a short flexor, and quadratus plantae, e.g., at about 1, 2, 3, 4, or 5 skin injection sites, preferably about 1-4 or about 1-3 sites into one or more of the subject muscles or fascia. For example, RT002 may be administered in this manner to obtain results provided herein.

In more preferred embodiments, specific dose amounts are injected into specific muscles or fascia at different depths of administration, via a single injection site. For example, in one embodiment, a fraction of a dose of about 50 U to about 120 U, about 60 U to about 100 U, about 70 U to about 90 U, or about 80 U per injection of the botulinum toxin component is injected in the plantar fascia, preferably the plantar fascia at the medial calcaneal tuberosity; and the remainder of the dose of the botulinum toxin component is injected into or near to the region immediately superior to the plantar fascia, preferably at or near the flexor digitorum brevis and/or the flexor hallucis longus. As another particular, a fraction of a dose of about 100 U to about 160 U, about 100 U to about 140 U, or about 120 U per injection of the botulinum toxin component is injected in the plantar fascia, preferably the plantar fascia at the medial calcaneal tuberosity; and the remainder of the dose of the botulinum toxin component is injected into or near to the region immediately superior to the plantar fascia, preferably at or near the flexor digitorum brevis and/or the flexor hallucis longus. The fraction for administration to the plantar fascia may be about ⅙, about ⅕, about ¼, about ⅓, about ½, about ⅔, or about ⅚ of the total injection dose, preferably about ⅓; with the remainder being about ⅚, about ⅘, about ¾, about ⅔, about ½, about ⅓, or about ⅙, preferably with the remainder being about ⅔.

In preferred embodiments, the therapeutic effect lasts for an extended duration of time for a higher proportion of individuals receiving the botulinum toxin pharmaceutical formulation compared with other botulinum toxin formulations. For example, administration of a pharmaceutical composition described herein may produce an improvement in plantar fasciitis that endures for at least about 4 weeks in 40-90% of individuals administered the formulation. In some embodiments, the response is maintained, or the effect endures, for at least about 4 weeks in at least over about 55%, over about 56%, over about 58%, over about 60%, over about 62%, over about 65%, over about 66%, over about 68%, over about 70%, over about 72%, over about 73%, or over about 75% of individuals each administered the pharmaceutical formulation, as described herein. In some embodiments, the response is maintained, or the effect endures, for at least about 16 weeks in at least over about 35%, over about 36%, over about 38%, over about 40%, over about 43%, over about 45%, over about 47%, over about 50%, over about 53%, over about 55%, over about 57%, over about 60%, over about 63%, over about 65%, over about 68%, more preferably over about 70%, over about 73%, or over about 75%, of individuals each administered the pharmaceutical formulation, as described herein. In some embodiments, the response is maintained, or the effect endures, for at least about 24 weeks in at least over about 15%, over about 16%, over about 18%, over about 20%, over about 22%, over about 23%, over about 25%, over about 27%, or over about 30%, of individuals each administered the pharmaceutical formulation, as described herein.

In general, methods and procedures for measuring the activity of botulinum toxin, i.e., units (U) of botulinum toxin activity, are known to and practiced by those having skill in the art. Briefly, median lethality assays (LD₅₀ assays) in mice are conventionally used to estimate the number of units of botulinum toxin with a high degree of precision. Doses of all commercially available botulinum toxins are expressed in terms of units of biologic activity. By way of example, one unit of botulinum toxin corresponds to the calculated median intraperitoneal lethal dose (LD50) in female Swiss-Webster mice. See, Hoffman, et al., 1986, Int. Ophthalmol. Clin., 26:241-50, as well as DePass, L. R., 1989, Toxicol. Letters, 49:159-170; and Pearce, L. B. et al., 1994, Toxicol. Appl. Pharmacol., 128:69-77, which also describe lethality assays in the art.

More particularly, a suitable method for determining botulinum toxin units for a botulinum toxin component of the compositions of the invention is as follows: Forty-eight (48) female CD-1 mice weighing 17-23 grams are randomly assigned to six doses of the test article (1.54, 1.31, 1.11, 0.95, 0.80, and 0.68 U/0.5 mL), eight (8) animals per dose group. The test article refers to the botulinum toxin preparation or sample being assayed or tested. The animals are housed eight per cage and are weighed within 24 hours of dosing with the test article. On the day of dosing, the test article is diluted to the appropriate concentrations in isotonic saline (0.9% NaCl). Each animal is administered 0.5 mL of diluted test article via intraperitoneal injection. After injection, mice are returned to the cage and fatalities are recorded daily for three days. Lethality is scored 72 hours post injection and the results are analyzed by probit or logistic analysis to derive the LD₅₀ value relative to a reference standard that is assessed using the same dosing regimen. By way of example, the reference standard is a specifically qualified and calibrated lot of the same composition of the invention that is used for comparison to derive relative potency of the test article. The determined LD₅₀ value is then corrected for the cumulative dilutions performed to assign a relative potency value for the neat (undiluted) test article.

Alternatives to LD₅₀ testing include assays using neuronal cell lines or endopeptidase assays, which avoid testing in animals (see, e.g., Sesardic et al., “Alternatives to the LD50 assay for botulinum toxin potency testing: Strategies and progress towards refinement, reduction and replacement” Proc. 6^{th} World Congress on Alternatives & Animal Use in the Life Sciences, Aug. 21-25, 2007, 14 Special Issue, pp 581-585). Such methods may be used, in addition or instead of LD₅₀ assays, for determining botulinum toxin units for a botulinum toxin component of the compositions of the invention.

Extended Duration

In another aspect, the invention provides methods and uses of the pharmaceutical formulations, described herein, to achieve an extended duration of effect. In preferred embodiments, formulations described herein are used to administer botulinum toxin to a subject in need thereof to produce an extended duration therapeutic effect compared to treatments using conventional botulinum toxin formulations. In some embodiments, the method comprises administering by injection a therapeutically effective dose of a sterile injectable formulation, as described herein, preferably into one or more muscles or fascia associated with plantar fasciitis, to achieve the extended duration therapeutic effect following the injection treatment. In some embodiments, the method comprises administering by topical application a therapeutically effective dose of a topical formulation, as described herein, to the skin overlying one or more muscles or fascia associated with plantar fasciitis, to achieve the extended duration therapeutic effect following the topical treatment. In preferred embodiments, administration of the botulinum toxin compositions results in an increased duration of effect, such as an improvement in at least one symptom of plantar fasciitis, or a related disorder, that lasts longer than treatment with conventional botulinum toxin formulations, thereby allowing lengthier intervals between treatments.

Duration of effect may be measured by any measure described herein and/or known in the art, or a combination thereof. For example, any one or more measures discussed in the Examples herein, in particular Example 1, for primary, secondary, and/or exploratory endpoints, may be used in assessing duration of effect, that is, the period for which the botulinum toxin composition shows effect in reducing one or more symptoms of plantar fasciitis, or a disorder related thereto, such as, the period of reduced pain, or preferably no pain, following treatment. In a particular embodiment, a reduction in plantar fasciitis pain may be considered to endure until the time the pain returns to baseline, before initial treatment; or may be considered to endure until one or more “points” of improvement on a pain scale is/are lost, following a treatment; or may be considered to endure as long as scores corresponding to none, mild, or tolerable pain are maintained, following a treatment, again based on one or more measures for assessing plantar fasciitis pain, as described herein.

Preferred embodiments afford a reduction in one or more plantar fasciitis symptoms for at least about 3 months through about 11 months, about 5 months through about 10 months, about 6 months through about 10 months, or for at least about 16 weeks through about 24 weeks. In particular embodiments, the duration of therapeutic effect is at least about 16 weeks, at least about 20 weeks, at least about 24 weeks or at least about six months, at least about 7 months, at least about 8 months, at least about 9 months, or at least about 10 months before a second or subsequent treatment dose is administered. One or more such results may be obtained in embodiments comprising administering by injection to one or more muscles or fascia associated with plantar fasciitis a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 200 U to about 300 U, preferably about 240 U per injection treatment; wherein the positively charged carrier is non-covalently associated with the botulinum component. In some such embodiments, administration comprises at least one injection into one or more muscles or fascia selected from the group consisting of plantar fascia, the gastrocnemius-soleus complex, periosteum, quadratus plantae, and short flexors. For example, in some of these embodiments, administration comprises about 5-7 injections of about 160 U into the gastrocnemius-soleus complex, e.g., at 2 cm-intervals; and about 80 U divided amongst the following four sites (1) the plantar fascia, preferably at the point of maximum tenderness associated with plantar fasciitis pain; (2) the periosteum, preferably the area over the plantar insertion, (3) the quadratus plantae; and (4) the short flexors. See also Example 1.

One or more such results also may be obtained in embodiments comprising administering by injection to a muscle or fascia associated with plantar fasciitis a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 50 U to about 200 U, about 60 to about 160 U, or about 80 U, about 100 U, or about 120 U in a single injection through one site; wherein the positively charged carrier is non-covalently associated with the botulinum component. In some such embodiments, administration comprises one injection into a muscle or fascia selected from the group consisting of plantar fascia, the flexor digitorum brevis, and the flexor hallucis longus. For example, in some of these embodiments, administration comprises a single injection of about 40 U into the plantar fascia, preferably at the medial calcaneal tubercle; and about 80 U into an area above the plantar fascia, preferably immediately superior to it, such as into or near to the flexor digitorum brevis and/or flexor hallucis longus, for administration of a total single dose of about 120 U. As another example, administration comprises a single injection of about 80×⅓ U into the plantar fascia, preferably at the medial calcaneal tubercle; and about 80×⅔ U into an area above the plantar fascia, preferably immediately superior to it, into or near to the flexor digitorum brevis and/or flexor hallucis longus, for administration of a total single dose of about 80 U.

Particularly preferred embodiments afford a therapeutic effect, in particular, a reduction in plantar fasciitis pain, for about 3 months through about 11 months, about 5 months through about 10 months, about 6 months through about 10 months, or about 20 weeks through about 40 weeks. In preferred embodiments, the duration of effect is at least about 22 weeks, at least about 24 week, at least about 26 weeks, at least about 28 weeks, at least about 30 weeks, at least about 32 weeks, at least about 34 weeks, at least about 36, weeks, at least about 38 weeks, at least about 40 weeks, or at least about 42 weeks, before a second or subsequent treatment dose is administered. In particular embodiments, the interval before administering a second or subsequent treatment dose of the composition is greater than or equal to 20 weeks, 22 weeks, 24 weeks, 26 weeks, 28 weeks, 30 weeks, 32 weeks, 34 weeks, 36 weeks, 38 weeks, 40 weeks, or greater than or equal to 42 weeks, following the initial treatment dose or following subsequent treatment doses. One or more of these results are obtained in embodiments comprising administering by injection to one or more muscles or fascia associated with plantar fasciitis a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)15-GRKKRRQRRR (SEQ ID NO: 4); wherein the botulinum toxin component is administered to the individual in a treatment dose amount of about 200 U to about 300 U, preferably about 240 U per injection treatment; wherein the positively charged carrier is non-covalently associated with the botulinum component. In some such embodiments, administration comprises about 5-7 injections of about 160 U into the gastrocnemius-soleus complex, e.g., at 2 cm-intervals; and about 80 U divided amongst the following four sites (1) the plantar fascia, preferably at the point of maximum tenderness associated with plantar fasciitis pain; (2) the periosteum, preferably the area over the plantar insertion, (3) the quadratus plantae; and (4) the short flexors. See also Example 1. One or more such results also may be obtained in embodiments comprising administering by injection to a muscle or fascia associated with plantar fasciitis a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)₁₅-GRKKRRQRRR (SEQ ID NO: 4); wherein the botulinum toxin component is administered to the individual in a treatment dose amount of about 50 U to about 200 U, about 60 to about 160 U, or about 80 U, about 100 U, or about 120 U in a single injection through one site; wherein the positively charged carrier is non-covalently associated with the botulinum component. In some such embodiments, administration comprises one injection into a muscle or fascia selected from the group consisting of plantar fascia, the flexor digitorum brevis, and the flexor hallucis longus. For example, in some of these embodiments, administration comprises a single injection of about 40 U into the plantar fascia, preferably at the medial calcaneal tubercle; and about 80 U into an area above the plantar fascia, preferably immediately superior to it, such as into or near to the flexor digitorum brevis and/or flexor hallucis longus, for administration of a total single dose of about 120 U. As another example, administration comprises a single injection of about 80×⅓ U into the plantar fascia, preferably at the medial calcaneal tubercle; and about 80×⅔ U into an area above the plantar fascia, preferably immediately superior to it, into or near to the flexor digitorum brevis and/or flexor hallucis longus, for administration of a total single dose of about 80 U.

In another aspect, the invention provides methods and uses of the pharmaceutical formulations, described herein, in a treatment regimen for plantar fasciitis or a disorder related thereto, where intervals between one or more successive treatments are longer than those in a treatment regimen for same using conventional botulinum toxin formulations, such as where multiple treatments are used to maintain a treatment goal and/or manage plantar fasciitis or related disorder. For example, the invention provides, in some embodiments, a method of treating an individual suffering from plantar fasciitis, where the method comprises a treatment course having multiple treatments with prolonged duration of therapeutic effect and, accordingly, lengthier intervals between successive treatments compared to regimens using conventional botulinum toxin formulations (i.e., formulations not containing a carrier molecule, as described herein). For example, products containing botulinum toxin without a carrier described herein typically provide an effect for less than 6 months, such as only for about 3-4 months.

In particular embodiments, the interval before administering a second or subsequent treatment dose of the botulinum toxin-containing composition is greater than or equal to at least about 20 weeks, at least about 22 weeks, at least about 24 weeks, at least about 26 weeks, at least about 28 weeks, at least about 30 weeks, at least about 32 weeks, at least about 34 weeks, at least about 36, weeks, at least about 40 weeks, or at least about 42 weeks, following the initial treatment dose or following subsequent treatment doses. A median duration between doses may be 23 weeks, at least 23 weeks, or greater than 23 weeks; 24 weeks, at least 24 weeks, or greater than 24 weeks; 25 weeks, at least 25 weeks, or greater than 25 weeks; 26 weeks, at least 26 weeks, or greater than 26 weeks; 27 weeks, at least 27 weeks, or greater than 27 weeks; 28 weeks, at least 28 weeks, or greater than 28 weeks; 30 weeks, at least 30 weeks, or greater than 30 weeks. Accordingly, in particularly preferred embodiments, an individual is administered a dose of a formulation described herein twice per year, or fewer times than twice a year, for example, every 7, 8, 9, 10, or 11 months, or one a year.

In preferred embodiments, methods and compositions of the present invention provide extended duration therapeutic effect in an individual suffering from plantar fasciitis, in which the method comprises administering by injection a dose of a sterile injectable formulation into one or more muscles or fascia associated with plantar fasciitis to achieve the therapeutic effect following treatment, preferably a first treatment. In more preferred embodiments, the composition comprises botulinum toxin A, such as botulinum toxin A of 150 kDa MW and a positively charged carrier comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2), or (gly)ₚ-RKKRRQRRR-(gly)_{q} (SEQ ID NO: 3), wherein the subscripts p and q are each independently an integer of from 0 to 20; preferably comprising the amino acid sequence RKKRRQRRRG-(K)15-GRKKRRQRRR (SEQ ID NO: 4). In still more preferred embodiments, the botulinum toxin is administered by injection to the individual in a single treatment dose in an amount that provides about 200 U to about 300 U; about 220 U to about 280 U, about 220 U to about 260 U, or about 240 U botulinum toxin. In even still more preferred embodiments, the botulinum toxin is administered by a single injection through one site in an amount that provides about 50 U to about 200 U, about 60 to about 160 U, or about 80 U, about 100 U, or about 120 U in the single injection administration. In a particular example, the pharmaceutical formulation further comprises a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5. to pH 7.5, in w/v amounts as described herein. In still more preferred embodiments, the formulation is albumen-free. In one particularly preferred embodiment, the pharmaceutical composition comprises 0.1 mg polysorbate 20, 36 mg trehalose dehydrate, and 11.7 μg RTP004, per 50 U of the 150 kDa type A toxin, and the treatment dose is 240 U. In another particularly preferred embodiment, the pharmaceutical composition comprises 0.1 mg polysorbate 20, 36 mg trehalose dehydrate, and 11.7 μg RTP004, per 50 U of the 150 kDa type A toxin, and the treatment dose is 80 U. In still another particularly preferred embodiment, the pharmaceutical composition comprises 0.1 mg polysorbate 20, 36 mg trehalose dehydrate, and 11.7 μg RTP004, per 50 U of the 150 kDa type A toxin, and the treatment dose is 120 U.

In preferred embodiments, a single treatment dose using one or more of the compositions defined above, reduces VAS pain or NPRS pain associated with plantar fasciitis by at least about 50% through week 8 following the treatment. More preferably, treatment regimens as described herein provide sustained relief from chronic heel pain and can support healing of the plantar fascia, without risks of plantar fascia rupture and/or atrophy of the fat pad, which often occur with repeated cortical injections.

Kits

This invention also contemplates the use of a variety of delivery devices for administering botulinum toxin-containing compositions described herein across skin in the treatment and management of plantar fasciitis or a disorder relating thereto. For injectable formulations, such devices may include, without limitation, a needle and syringe, or may involve more sophisticated devices capable of dispensing and monitoring the dispensing of the composition, and optionally monitoring the condition of the subject in one or more aspects (e.g., monitoring the reaction of the subject to the substances being dispensed).

This invention also contemplates devices for transdermal delivery of the topical formulations described herein. Such devices may be as simple in construction as a skin patch, or may be more complicated devices that include means for dispensing and monitoring the dispensing of the composition, as described above.

It should be noted that the choice of materials for the construction of the device is important. Preferred materials for the construction of delivery devices are those that do not lead to a loss of activity of the botulinum toxin/carrier composition, either through degradation or unwanted adsorption of the botulinum toxin on a surface of the device. Such undesired behavior has been observed, for example, when botulinum toxin/carrier in an aqueous solution contacts polypropylene surfaces, but not when the botulinum toxin/carrier solution contacts polyvinyl chloride (PVC) surfaces.

In some embodiments, the compositions can be pre-formulated and/or pre-installed in a delivery device. This invention also contemplates embodiments wherein the compositions are provided in a kit that stores one or more components separately from the remaining components. For example, in certain embodiments, the invention provides for a kit that separately stores the botulinum toxin component and the carrier in separate containers (e.g., first and second containers) for combining at or prior to the time of application. The amount of carrier to botulinum toxin will depend on which carrier is chosen for use in the composition in question.

For example, the amount of carrier to botulinum toxin may be provided in a ratio selected from the group consisting of about 0.01 μg/U, about 0.02 μg/U, about 0.04 μg/U, about 0.06 μg/U, about 0.08 μg/U, about 0.1 μg/U, about 0.12 μg/U, about 0.14 μg/U, about 0.15 μg/U, about 0.16 μg/U, about 0.18 μg/U, about 0.20 μg/U, about 0.22 μg/U, about 0.23 μg/U, about 0.234 μg/U, about 0.24 μg/U, about 0.25 μg/U, about 0.26 μg/U, about 0.28 mgc/U, about 0.3 mgc/U, about 0.32 mgc/U, about 0.34 mgc/U, about 0.36 mgc/U, about 0.38 mgc/U, or about 0.4 μg per U of botulinum toxin, preferably where the carrier is RTP004. In particular embodiments, botulinum toxin is provided in an amount of about 240 U (referring to the 150 kDa toxin protein molecule of type A) and the RTP004 carrier is provided an amount of about 54 μg, about 55 μg, about 56 μg, about 57 μg, about 58 μg, about 59 μg, or about 60 μg. For example, as provided above, in some particular embodiments, the carrier is RTP004 and is provided at about 12 μg per 160 U, at about 18 μg per 240 U, or at about 45 μg per 600 U of the 150 kDa botulinum toxin molecule.

The invention also contemplates approaches for administering the botulinum toxin component to a subject or patient in need thereof, in which a therapeutically effective amount of botulinum toxin is administered in conjunction with a carrier, as described herein. By “in conjunction with” it is meant that the two components (botulinum toxin and carrier) are administered in a combination procedure, which may involve either combining them prior to administration to a subject, or separately administering them, but in a manner such that they act together to provide the requisite delivery of a therapeutically effective amount of the toxin. The botulinum toxin may be stored in dry form in a syringe or other dispensing device and the carrier may be injected or topically applied before application of the toxin so that the two act together, resulting in the desired tissue penetration enhancement and/or other improved characteristics over conventional botulinum toxin formulations, as detailed above. In that sense, the two substances (carrier and botulinum toxin) act in combination or perhaps interact to form a composition or combination in situ. Accordingly, the invention also includes a kit with a device for dispensing botulinum toxin and a liquid, gel, or the like, that contains the carrier and that is suitable for topical application or injection to the target tissue. Kits for administering the compositions of the inventions, either under direction of a health care professional or by the patient or subject, may also include a custom applicator suitable for that purpose.

It is understood that the following examples and embodiments described herein are for illustrative purposes and that various modifications or changes in light thereof will be suggested to persons skilled in the art and are to be included within the spirit and purview of this application and scope of the appended claims.

All publications, patents, and published patent applications cited herein are hereby incorporated by reference in their entireties for all purposes.

### EXAMPLES

### Example 1

### Injectable Botulinum Toxin Formulation in the Treatment of Plantar Fasciitis

This Example describes a clinical study to compare to placebo the safety and efficacy, in managing plantar fasciitis, of a single administration of an injectable composition of the invention, referred to as RT002, containing botulinum toxin A and a positively charged carrier. The RT002 product is an injectable formulation, which contains the 150 kD subtype A botulinum toxin molecule (the active or active ingredient), which is non-covalently associated with a positively charged carrier peptide having the formula RKKRRQRRRG-(K)₁₅-GRKKRRQRRR (SEQ ID NO: 4), and which does not contain accessory proteins or animal-derived components. The dose is 240 U of RT002. RT002 also is referred to herein as DaxibotulinumtoxinA for Injection or (Daxi for Injection).

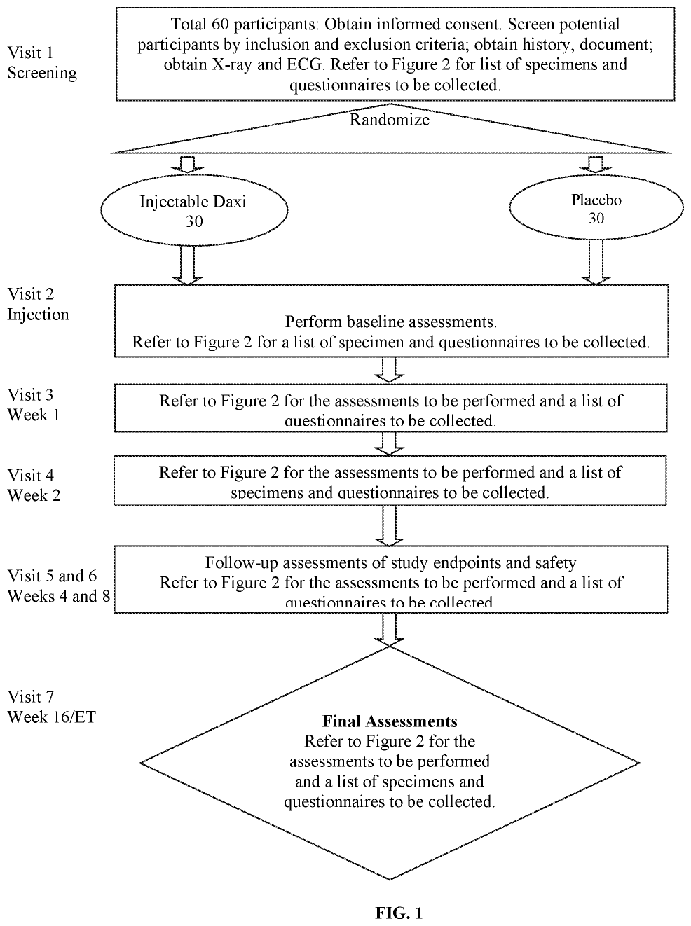

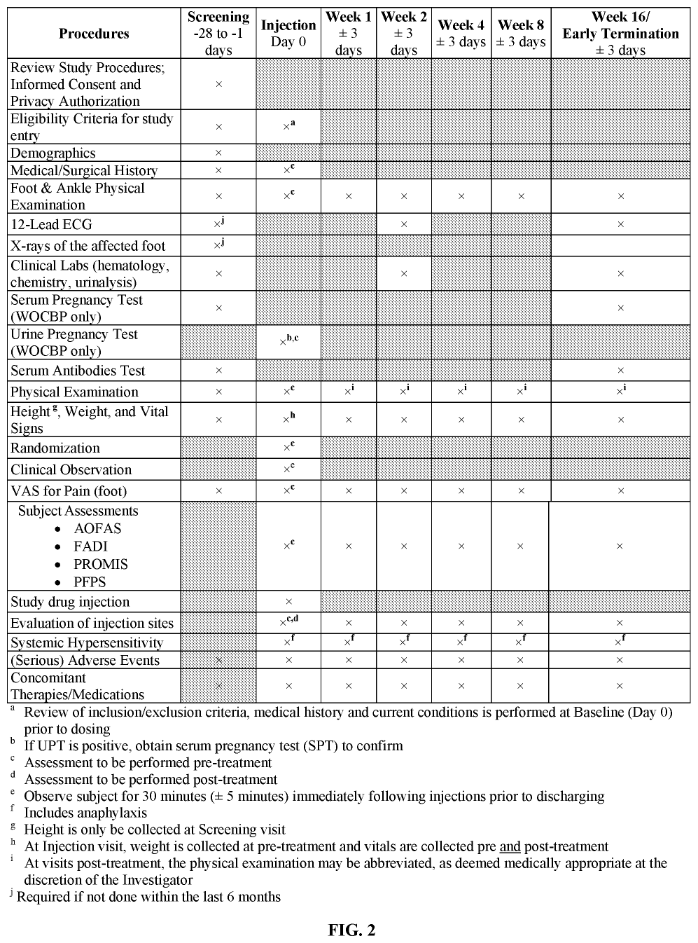

The clinical study was a phase 2, prospective, randomized, double blinded, placebo-controlled trial, lasting about 8 months and about 5 months per participant (up to 4 weeks of screening, a single day of treatment, and up to 16 weeks of follow up). Fifty nine male or female subjects were randomly assigned to one or two study groups. Group 1 received 7.5 mL (240 U) in a single dose; while Group 2 received 7.5 mL placebo in a single dose. The duration of effect of a single treatment of RT002 at this dosage also was assessed. See FIG. 1 and FIG. 2. The study aimed to use transient and selective partial paralysis of the gastrocnemius/soleus complex, combined with a simple home program and inexpensive splinting, to ameliorate signs and symptoms of plantar fasciitis, improve function, and optimize health related quality of life.

The RT002 product is composed of purified 150 kDa botulinum neurotoxin, referred to as RTT150, formulated in a lyophilized powder, as well as containing a positively charged carrier, RTP004. RTT150 is a purified form of the neurotoxin, free of accessory proteins and containing no pooled human serum albumin, bacterial hemagglutins, or other human or animal derived components. This makes RTT150 free of the risk of prion-based and blood-based diseases. RTT150 comprises 160 U RTT150 toxin, per 2 mL vial. Along with RTP004, other components of the formulation include trehalose dihydrate, L-histidine, L-histidine hydrochloride, and polysorbate 20. RTT150 was packaged in 2 mL clear type 1 borosilicated glass single-use vials that are stoppered, oversealed, and stored at 2-8° C. The product was provided in single-use vials of 160 U of sterile vacuum-dried powder for reconstitution, refrigerated during transit.

When provided in a 50 U vial, in lyophilized form, the vial contains 0.1 mg polysorbate 20, 36 mg trehalose, and 11.7 μg RTP004 as the carrier, to give a mass ratio of carrier:toxin of 51,000:1. The 11.7 μg RTP004 may also be used with other amounts of toxin as the ratio in mass of carrier to toxin is in the order of 25,000:1.

The product was reconstituted with 5 mL sterile, non-preserved 0.9% sodium chloride solution (saline), and stored at 2-8° C. until use within 2 hours of preparation. The placebo control is a lyophilized product of components other than the botulinum toxin, provided in single-use vials, and was reconstituted in the same manner. The product was prepared by a trained dose preparer (unblinded) prior to use and was administered by a physician (blinded) into the muscles (IM) and deep plantar fascia. The appearance of RT002 and the placebo was the same, both in the vial before and after reconstitution, and in the syringe (clear and colorless solution).

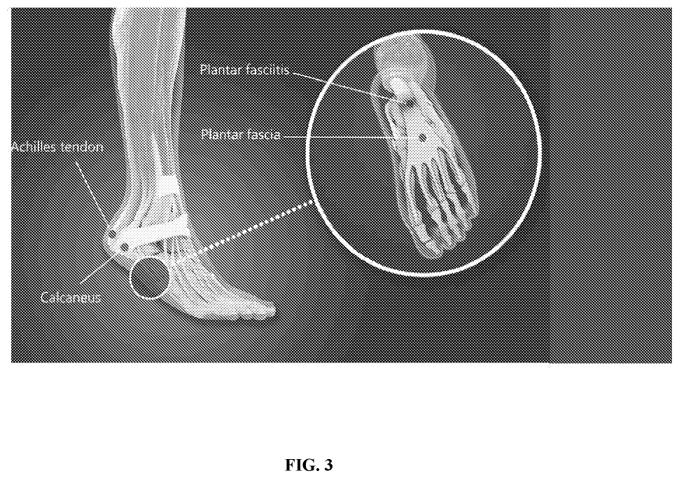

Five (5) mL (160 U) of the reconstituted solution in the syringe were injected into the triceps surae (gastrocnemius-soleus complex) at 2 cm intervals, in 5-7 sites, depending on leg length/mass. Two and a half (2.5) mL (80 U) of the reconstituted solution were injected into four (4) sites (the point of maximum tenderness within the plantar fascia, the periosteum over the plantar insertion, the quadratus plantae, and the short flexors) through one (1) to three (3) insertion skin sites. Ultrasound guidance was used for placement of injections to confirm placement in the target structure. Administration was to the symptomatic extremity. A total dose of 240 U was administered using a needle size of at least 27 gauge in diameter and not exceeding 1.5 inches in length. See FIG. 3.

Nonclinical Evidence of Safety of Components: Nonclinical testing of RTT150 (toxin) with RTP004 (carrier) showed the expected dose dependent neuromuscular effects when dosed systemically (SC, IM), without any unexpected systemic or local toxicity. These studies included GLP and non-GLP single dose, repeat dose, and reproductive Segment II toxicity studies in rodents and non-rodent species. RTT150 was tolerated after repeat dose IM administration of up to 50 U/kg in rats. Specifically, when the antigenicity of the components RTT150 (toxin) and RTP004 (peptide) were studied in repeat-dose nonclinical studies, delivered topically or systemically, there was no meaningful increase in titers against either RTT150 or RTP004 in blood samples collected within any nonclinical study, including studies involving 1 month mini-pigs (dermal delivery), 3-month mice (SC injection), 3-month rats (dermal delivery), 6-month rats (IM injection) with 3 month recovery, and 9-month rabbits (dermal delivery) with 3 month recovery. RTP004 was dosed at maximum feasible dose without effect in dermal, genotoxicity, and reproductive toxicity studies and produced no significant findings in parenteral studies at a safety multiple of more than 9,500-fold. Across studies, the topical and injectable formulations provide reassuringly safe profiles. In one previous clinical trial for glabellar lines, one subject had a positive result for anti-RT002 antibody, both at baseline and at the end of the study.

Dosing regimen and injection technique: The dosing regimen of RT002 for this study was a single treatment of either RT002 (240 U) or placebo, consisting of injection of plantar muscles and fascia. Study subjects received a single treatment of 7.5 mL per injection. Investigators, site staff, subjects, and the sponsor were blinded to the treatment group assignments.

Study population: Approximately 60 adult, female and male subjects, 18-65 years of age and in good general health, were enrolled in the study. The subjects had moderate to severe unilateral plantar fasciitis at entry, with persistent heel pain for more than 3 months (such as pain with walking, pain interfering with quality of life, pain greater than 45/100 on the VAS for pain in the morning, especially with the first steps out of bed), and the plantar fasciitis had not responded to conservative treatment modalities, for example, anti-inflammatory medications, splinting, heel pads, stretching exercises, and/or steroid injections, for at least 3 months. Diagnosis was made by physical examination and/or ultrasonography. Unilateral plantar fasciitis means that the subject has presented no symptoms or signs in the contralateral foot and has not sought medical attention for the contralateral foot within 3 months of diagnosis. X-rays of the affected foot were made to rule out concomitant foot pathology.

Table 1 provides additional details regarding disposition and analysis of the subject populations; Table 2 provides additional details regarding demographics of the populations.

TABLE 1

| | Placebo | Daxi 240 U | All Subjects |
|---|---|---|---|
| Subject Disposition | (n = 29) | (n = 30) | (n = 59) |
| Completed Week 8 | 26 (89.7%) | 26 (86.7%) | 52 (88.1%) |
| Completed the Study | | | 15 (25.4%) |
| at Week 16 | | | |
| Lost to Follow up | | | 3 (5.1%) |
| Early Termination | | | 5 (8.5%) |
| Analysis Populations | | | |
| Intent-to-treat (ITT) | 29 (100%) | 30 (100%) | |
| Safety | 29 (100%) | 30 (100%) | |

TABLE 2

| | Placebo | Daxi 240 U |
|---|---|---|
| | (n = 29) | (n = 30) |
| Female, n (%) | 22 (75.9%) | 20 (66.7%) |
| Age(years), mean (SD), | 46.9 (9.6) | 46.9 (9.2) |
| range | 28 to 64 | 28 to 61 |
| Hispanic/Latino, n (%) | 6 (20.7%) | 2 (6.7%) |
| Race: | | |
| White | 20 (69.0%) | 25 (83.3%) |
| Black/African American | 8 (27.6%) | 5 (16.7%) |

All subjects: prior analgesics = 1 (1.7%); prior anti-inflammatory/anti-rheumatic = 2 (3.4%)

The 60 subjects were randomly assigned to one of two study groups: Group 1 (n=30) received injections with the active agent; and Group 2 (n=30) received injections with the placebo (vehicle-only). It was determined that this sample size provides sufficient power to detect differences between RT002 and placebo treatments in terms of primary outcome of improvement, as well as providing sufficient data to ascertain safety and efficacy with a high probability of clinical significance (p<0.05).

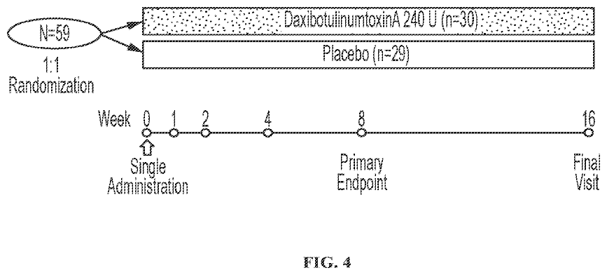

Visit Schedule: A screening visit was conducted up to four (4) weeks prior to randomization, and subjects were treated with RT002 or placebo on Injection Day. Post-treatment on-site follow-up visits occurred at Weeks 1, 2, 4, 8, and 16, or any time a subject terminated early. Acceptable study visits were within 3 days, before or after the scheduled visit. See FIG. 4.

Subjects were instructed to wear night splints daily after the injection treatment, and were questioned about compliance every study visit. Standardized home therapy program also included Achilles tendon and plantar fascia stretching exercises.

Injection Site Evaluation: The injection site was evaluated at Day 0 (Injection Visit), pre- and post-treatment, and at follow up visits on weeks 1, 2, 4, 8, and 16, or early termination visit, if applicable). The injection site was assessed for erythema, edema, burning or stinging, itching, bruising, and/or drainage. Any adverse events were recorded, as applicable.

Foot and Ankle Examination: Examination for the foot included ankle, toe, and subtalar range of motion, foot motor strength, location of pain, and examination of the heel fat pad and Tinel's sign. Evaluation was done at Screening, pre-treatment Injection, Weeks 1, 2, 4, 8, and 16/Early Termination visits. The presence of toe deformities, bunions, ulcers, and/or sores was documented. The feet also were examined for signs of swelling, pitting edema, infection, or vascular abnormalities.

Criteria for Evaluation: Primary Objective: to compare the safety and efficacy of RT002 versus placebo for managing plantar fasciitis. Primary safety endpoint: adverse events associated with the two study treatments. Primary efficacy evaluation (outcome endpoint): reduction in the visual analog scale (VAS) for pain for the foot at week 8. This is a clinically relevant outcome measure for plantar fasciitis.

Secondary Objectives: to evaluate the impact of RT002 on function and quality of life. Secondary efficacy endpoint: reduction in VAS for pain for the foot at time points other than Week 8; American Orthopaedic Foot and Ankle Score (AOFAS) at every time point; improvement in Foot and Ankle Disability Index (FADI) at every time point; Patient Reported Outcome Measurement Information Study (PROMIS) at every time point. These are clinically relevant outcome measures for this indication. The combination provides clinically meaningful measurements of RT002's ability to reduce the pain associated with plantar fasciitis.

Exploratory Efficacy Evaluation: To evaluate the impact of RT002 on function and quality of life. Endpoint: Improvement in the Plantar Fasciitis Pain and Disability Scale (PFPS) at every time point. This also is a clinically relevant outcome measure for this indication.

Assessment of adverse events: An AE is any untoward medical occurrence (e.g., sign, symptom, disease, syndrome, intercurrent illness, clinically significant abnormal laboratory finding, injury, or accident) that emerges or worsens following administration of the investigational product and until the end of trial participation that may not necessarily have a causal relationship to the administration of the investigational product. An AE can therefore be any unfavorable and/or unintended sign (including a clinically significant abnormal laboratory result), symptom, or disease temporally associated with the use of an investigational product, whether or not considered related to the investigational product. A treatment-emergent AE is one that occurs after any period of exposure to treatment.

Adverse Events (AEs) were graded as mild, moderate, or severe. Mild: Event may be noticeable to subject; does not influence daily activities; usually does not require intervention. Moderate: Event may be of sufficient severity to make subject uncomfortable; performance of daily activities may be influenced; intervention may be needed. Severe: Event may cause severe discomfort; usually interferes with daily activities; subject may not be able to continue in the trial; treatment or other intervention usually needed

Pre-existing conditions, which increase in frequency, or severity or a change in nature as a consequence of an investigational product use also are considered an adverse event.

An unexpected AE is an adverse reaction, the nature or severity of which is not consistent with the applicable product information. Any clinically significant change in the study safety evaluations (e.g., vital signs, laboratory results, ECG, injection site evaluation, physical/neurological examinations, etc.) post-treatment are reported as an AE.

A serious adverse event (SAE) is any untoward medical occurrence that results in any of the following outcomes: death; life-threatening; persistent or significant disability/incapacity or substantial disruption of the subject's ability to carry out normal life functions; requires in-patient hospitalization or prolongs hospitalization; congenital anomaly/birth defect (i.e., an adverse outcome in a child or fetus of a subject exposed to the investigational product before conception or during pregnancy); does not meet any of the above serious criteria but based upon appropriate medical judgment may jeopardize the subject or may require medical or surgical intervention to prevent one of the outcomes listed above.

AEs were evaluated at the Injection visit post-treatment, follow-up Weeks 1, 2, 4, 8, and 16 or Early Termination visits, if applicable. These may include: generalized loss of strength and muscle weakness, difficulty swallowing, breathing or speaking, extreme tiredness, loss of bladder control, or symptoms of allergic reaction (rash, itching, etc.). Other potential botulinum toxin A AEs include: accommodation disorder, eyelid function disorder, areflexia, eyelid ptosis, aspiration, facial palsy, blurred vision, facial paresis, botulism, fourth cranial nerve paresis, bradycardia, gastrointestinal disorders, brow ptosis, headaches, bulbar palsy, hemiparesis, constipation, hypoglossal nerve paresis, cranial nerve palsies, hyporeflexia, cranial nerve paralysis hypotonia, diaphragmatic paralysis, monoparesis, diplopia, muscular weakness, dry mouth, paralysis, dysarthria, paralysis flaccid, dysphagia, paralytic ileus, dysphonia, paraparesis, dyspnea, paresis, extraocular muscle paresis, paresis cranial nerve, peripheral nerve palsy, peripheral paralysis, pelvic floor muscle weakness, pneumonia aspiration, pupillary reflex impaired, quadriparesis, respiratory arrest, respiratory depression, respiratory failure, speech disorder, third cranial nerve paresis, trigeminal nerve paresis, urinary retention, vocal cord paralysis, vocal cord paresis, xerophthalmia (dry eyes).

Statistical Methods: With a total of 60 subjects (30 per group) and assuming a drop-out rate of no more than 20%, the study had a 90% power to detect a difference between groups at a significant level of 0.05 based on a 2-sided two-sample t-test when the true effect size is at least 0.96 (i.e., if the true difference between groups is at least 0.96 of the standard deviation). Based on data reported in literature (Brook, et al. “Pulse Radiofrequency Electromagnetic Field Therapy: A Potential Treatment of Plantar Fasciitis,” Journal of Food & Ankle Surgery. 2012 (1-5); Elizondo-Rodriguez, et al. “A comparison of botulinum toxin A and intralesional steroids for the treatment of plantar fasciitis: a randomized, double-blinded study,” Foot Ankle Int. 2013 January; 34(1):8-14; Huang et al. “Ultrasonographic guided botulinum toxin type A treatment for plantar fasciitis: an outcome-based investigation for treating pain and gait changes,” J Rehabil Med. 2010 February; 42(2):136-40), the standard deviation for the VAS for pain for the foot in subjects with plantar fasciitis can be approximated by 3.1 points out of 0-10 scale (or equivalent to 31 points out of 0-100 scale). The placebo effect on pain score reduction can be approximated by at most 1.32 points (Brook, 2012) and the treatment effect with a botulinum toxin A agent on pain score reduction at two (2) months can be approximated by 5.5 points (Elizondo-Rodriguez, 2013). Therefore, it was reasonable to assume the true effect size of >0.96 for the study in comparing the mean reduction in the VAS for pain at eight (8) weeks between DaxibotulinumtoxinA for Injection (RT002) and placebo.

All evaluable efficacy data were included in the analysis following the intent-to-treat (ITT) principle. All subjects who received the study treatment (DaxibotulinumtoxinA for Injection or placebo) comprised the modified-ITT population and were grouped according to each subject's randomization assignment. The primary analysis focused on the reduction from baseline in the VAS for pain at eight (8) weeks with missing data imputed by the last available value prior to the visit with missing value, that is, the last-observation-carried-forward (LOCF) approach. Analysis of covariate (ANCOVA) model including treatment group as a factor and baseline pain score as a covariate were used.

Reductions from baseline VAS for pain for the foot over time up to eight (8) weeks also were analyzed using a statistical method that handled repeated measures such as a generalized linear model (GLM) including treatment group, time (visit) and the treatment-visit interaction term as factors and the baseline VAS for pain as a covariate. For other secondary and exploratory efficacy outcome measures, appropriate statistical models (e.g., ANCOVA or GLM for continuous variables, and chi-squared/Fisher's exact test or logistic regression for dichotomy or categorical variables) adjusting for relevant covariates were employed to evaluate the treatment effect.

As a sensitivity analysis to check the impact of major protocol violations to study results, the primary and secondary efficacy outcome measures also were analyzed on data from all subjects in per-protocol (PP) population, which consisted of subjects in the modified-ITT population who do not have any major protocol violations. To check the robustness of results using the LOCF approach, different methods (such as observed data only or multiple imputation) were employed to handle missing data when appropriate.

As an exploratory analysis, proportion of subjects achieving certain status defined by some efficacy assessments (e.g., having a reduction in the VAS for pain by a certain clinically meaningful cutoff-point) also were compared between treatment groups.

Safety data from all subjects who received study treatment was summarized with subjects being grouped based on the treatment each subject actually received.

Safety endpoints were analyzed (e.g., as summary statistics during treatment and/or as change scores from baselines). AEs were coded in accordance with Medical Dictionary for Regulatory Activities (MedDRA)), calculated (e.g., each AE will be counted once only for a given participant), presented (e.g., severity, frequency, and relationship of AEs to study intervention were presented by System Organ Class (SOC) and preferred term groupings) and information reported about each AE (e.g., start date, stop date, severity, relationship, expectedness, outcome, and duration).

Logistic regression for analysis covariance was used to analyze secondary outcome variables; univariate analysis using Fisher's Exact Test was used to analyze secondary variables that were categorical data. Logistic regression or t-tests were used to analyze secondary variables that are continuous measures.

Follow-Up: Subjects were evaluated five times after their injections: one week, two weeks, four weeks, eight weeks, and sixteen weeks. Acceptable study visit windows were ±3 days. At each visit, the patient completed the AOFAS, PROMIS, FADI, and the visual analog pain scores, and the physician examined both of their feet.

End of Study Definition: A participant was considered to have completed the study if he/she completed all visits of the study including the last visit or the last scheduled procedure shown in FIG. 2's Schedule of Assessments (SoA). The end of the study is defined as completion of the last visit or procedure shown in the SoA in the trial globally.

Study Results: Preliminary 8-week Phase 2a results for RT002 in treating plantar fasciitis were as follows.

Efficacy Data

Primary endpoint results for VAS for pain scores at Week 8 are depicted in FIG. 5. Results were based on data from an intent-to-treat (ITT) population analyzed by an ANCOVA model adjusting for center and baseline VAS scores with the last-observation-carried-forward (LOCF) approach.

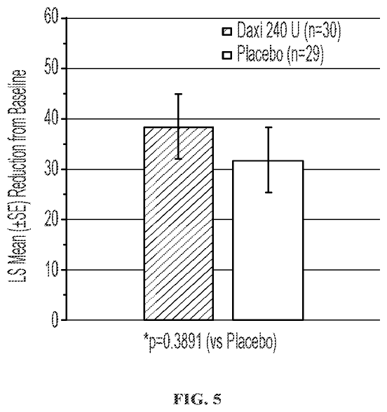

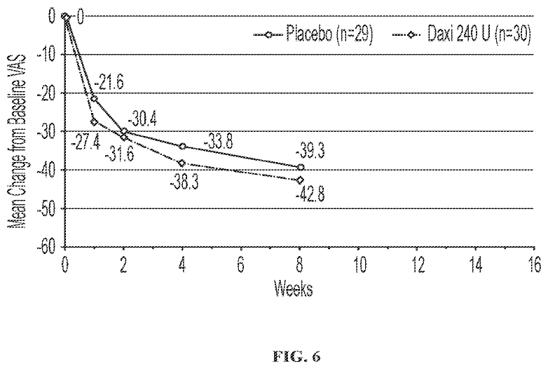

Secondary endpoint results for change in VAS for pain scores over time are depicted in FIG. 6, where the reduction in VAS scores observed beginning at Week 1 continued through Week 8 for both test and placebo treatment groups. Results were based on only observed data for subjects in the intent-to-treat (ITT) population.

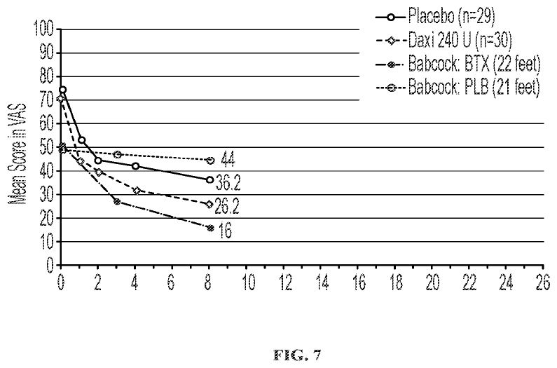

Secondary endpoint results for change in VAS for pain scores over time are depicted in FIG. 7, based on only observed data for subjects in the intent-to-treat (ITT) population, and further compared with results using botulinum toxin formulations from Babcock et al, 2005, Am J Phys Med Rehabil., 84(9): 649-54.

Secondary endpoint results for change in VAS for pain scores over time are depicted in FIG. 8, based on only observed data for subjects in the intent-to-treat (ITT) population, and further compared with results using botulinum toxin formulations and steroids from Elizondo-Rodriguez, et al. “A comparison of botulinum toxin A and intralesional steroids for the treatment of plantar fasciitis: a randomized, double-blinded study,” Foot Ankle Int. 2013 January; 34(1):8-14.

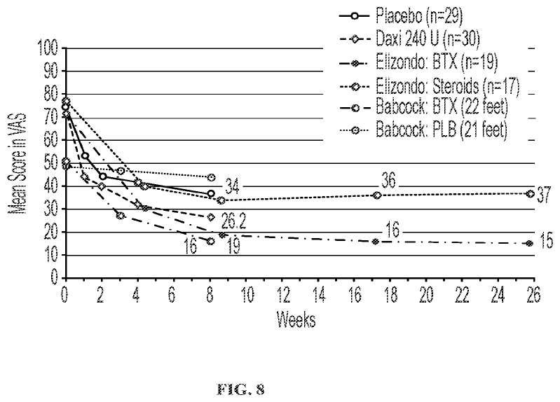

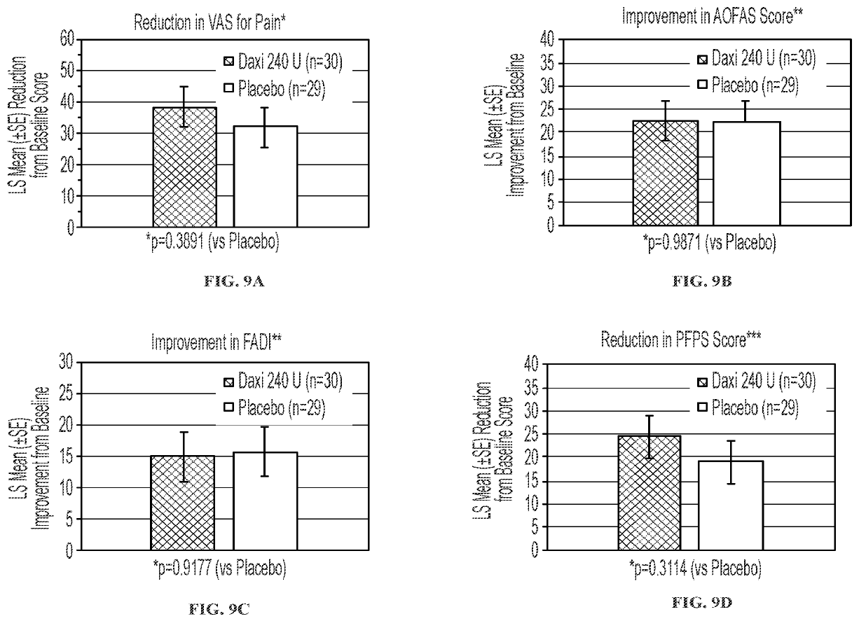

Efficacy endpoints at Week 8 based on different measures are depicted in FIGS. 9A-9D. Results were based on data for subjects in the intent-to-treat (ITT) population analyzed by an ANCOVA model adjusting for center and baseline VAS scores with the last-observation-carried-forward (LOCF) approach.

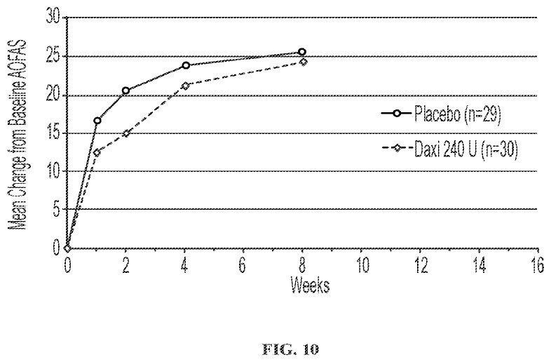

Secondary endpoint results for change in AOFAS over time are depicted in FIG. 10, based on only observed data for subjects in the intent-to-treat (ITT) population.

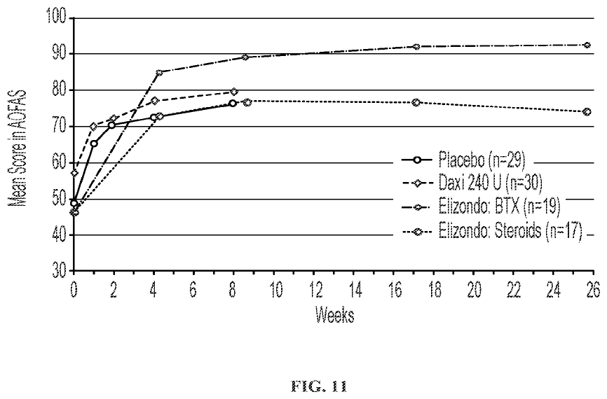

Secondary endpoint results for change in AOFAS over time are depicted in FIG. 11, based on observed data for subjects in the intent-to-treat (ITT) population, and further compared with results using other botulinum toxin formulations and steroids from Elizondo-Rodriguez, 2013.

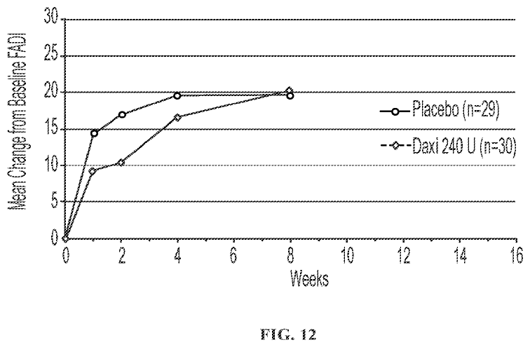

Secondary endpoint results for change in FADI over time are based on FIG. 12, based on only observed data for subjects in the intent-to-treat (ITT) population.

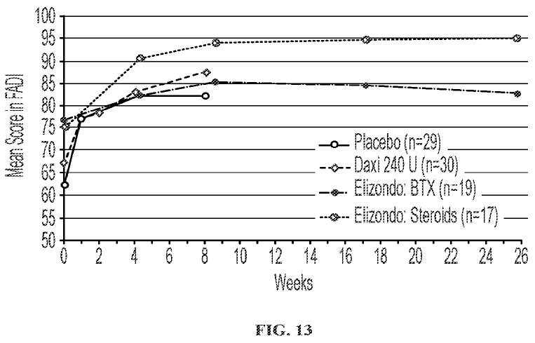

Secondary endpoint results for change in FADI over time are depicted in FIG. 13, based on observed data for subjects in the intent-to-treat (ITT) population, and further compared with results using other botulinum toxin formulations and steroids from Elizondo-Rodriguez, 2013.

The trial's primary endpoint, the reduction in the patient-reported visual analog scale (VAS) for pain at Week 8, showed a robust impact on pain, with a greater than 50% reduction for patients treated with RT002. In the intent to treat population, a mean reduction in the VAS score of 54.2% from baseline was achieved with RT002, compared with a 42.6% reduction in the placebo group. While not statistically significant (p=0.39), RT002 did outperform placebo, providing patients with considerable pain relief. Similar numeric trends were seen in the secondary and exploratory endpoints. The trial's secondary endpoints were (1) reduction in the VAS for pain in the foot, at time points other than Week 8; (2) change in AOFAS over time; and (3) improvement in FADI over time. Exploratory endpoint was improvement in the PFPS score over time.

This initial proof-of-concept study demonstrates that RT002 may have a positive impact on the severe heel pain caused by plantar fasciitis.

Regarding the high placebo response observed, this may have been due to one or more of the following: site-specific variability, spontaneous improvement, injection method, ongoing supportive treatments or additional undeclared treatments (e.g., use of ongoing treatment modalities like splints, orthotics, and pain medication), or other factors including regression to the mean, natural variation in disease severity, and response bias (see, e.g., Abhishek, et al., “Mechanisms of the placebo response in pain in osteroarthritis,” Osteoarthritis and Cartilage, 2013, 21(9):1229-35).

Plantar fasciitis is considered a self-limiting condition with symptoms resolving in 80-90% of cases within 10 months with conservative management, such as special insoles, physiotherapy, night splints, anti-inflammatory medicines, and losing weight (League, et al., “Current Concepts Review: Plantar Fasciitis,” Foot and Ankle International, 2008, 29(3):358-66). Nonetheless, the time to resolution of plantar fasciitis and associated symptoms can extend beyond 22 months, according to a survey of 100 people treated conservatively with average follow up of 47 months, where 82 were found to have resolution of symptoms (Landork, et al., “Plantar Heel Pain and Fasciitis,” Clinical Evidence 2006). In the present study, subjects were selected who failed conservative treatment for at least three months and were instructed to wear night splints daily after study injection.

Further analyses of the data were conducted.

Table 3 shows analysis of the subject population based on prior or concomitant use of analgesics or anti-inflammatory medications.

TABLE 3

| | Placebo | Daxi 240 U | All |
|---|---|---|---|
| | (n = 29) | (n = 30) | Subjects |
| Prior analgesics, n (%) | — | — | 1 (1.7%) |
| Concomitant analgesics, n (%) | 5 (17.2%) | 7 (23.3%) | 12 (20.3%) |
| Prior anti-inflammatory/ | 1 (3.4%) | 1 (3.3%) | 2 (3.4%) |
| anti-rheumatic, r (%) | | | |
| Concomitant | 10 (34.5%) | 12 (40.0%) | 22 (37.3%) |
| anti-inflammatory/ | | | |
| anti-rheumatic, n (%) | | | |

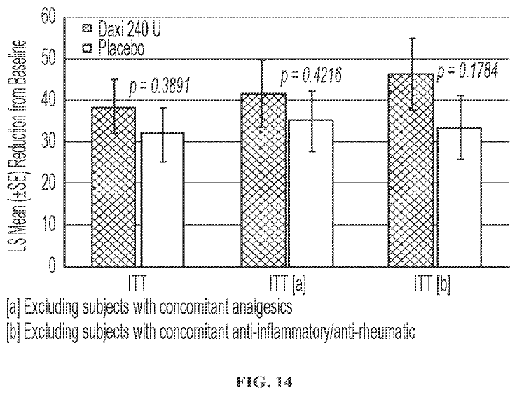

FIG. 14 depicts the results of two sensitivity analyses that were performed to assess impact of analgesia and anti-inflammatory medication on VAS for pain outcome measure at Week 8, the primary endpoint measure. Group [a] excludes subjects using concomitant analgesic medication; Group [b] excludes subjects using anti-inflammatory or rheumatic medication.

Table 4 summarizes randomized controlled studies comparing use of botulinum toxin type A to placebos, where all three placebo-controlled studies with botulinum toxin type A used treatment injections into the foot only. In these studies, the placebo arm had only small improvements in VAS Scores over time (Babcock et al, 2005, Am J Phys Med Rehabil., 84(9):649-54; Huang et al, 2010, J Rehabil Med., 42(2):136-40; and Ahmad et al, 2017, Foot Ankle Int., 38(1):1-7.)

Table 5 summarizes randomized controlled studies comparing use of botulinum toxin type A to steroids, where one study (Elizondo-Rodriguez, et al. “A comparison of botulinum toxin A and intralesional steroids for the treatment of plantar fasciitis: a randomized, double-blinded study,” Foot Ankle Int. 2013 January; 34(1):8-14) used treatment injections into the calf, resulting in significant decreases in VAS scores from month 1 to month 12, and a different study used treatment injections into the arch of the foot (Diaz-Llopis IV et al, 2013, Clin Rehabil., 27(8):681-5).

FIG. 7, referred to above, depicts secondary endpoint results for change in VAS for pain over time, based on only observed data for subjects in the ITT population, and further compares these results with the results summarized in Table 4 and Table 5.

TABLE 4

| Study | Study | Injection | | | | | |
|---|---|---|---|---|---|---|---|
| Name | Design | site | Duration | VAS for pain | | | |
| Babcock et al. 2005 | R, DB, PC | 40 U to the | 8 weeks | | Baseline | 3 | 8 W |
| Botox vs. Placebo | N = 27 | calcaneus and | | | | | |
| | Botox: 70 U divided | 30 U in the | | | | | |
| | in 2 doses | arch of the foot | | | | | |
| | | | | Botox | 5.1(2.0-9.7) | 2.7(0-7.9) | 1.6(0-7.9) |
| | | | | | | P <0.001 | P <0.001 |
| | | | | Pbo | 4.9(1.0-9.7) | 4.7(2.2-9.8) | 4.4(2.0-9.8) |
| | | | | | | P = NS | P = NS |
| Huang et al. 2010 | R, DB, PC | Injected into | 3 months | | Baseline | 3 W | 3 M |
| Botox vs. Placebo | N = 50 | the plantar fascia | | | | | |
| | Botox: 50U | of the foot | | | | | |
| | | | | Botox | 5.9(0.9) | 3.4(1.0) | 2.0(1.2) |
| | | | | | | P <0.001 | P <0.001 |
| | | | | Pbo | 5.4(0.6) | 5.1(0.8) | 5.2(1.0) |
| | | | | | | P = NS | P = NS |
| Ahmad J et al. 2016 | R, DB, PC | 100 U to the | 12 months | | Baseline | 6 M | 12 M |
| Botox vs Placebo. | N = 25 | calcaneus | | | | | |
| | Xeomin 100U | of the foot | | | | | |
| | | | | Botox | 7.2(6-10) | 3.6(0-8) | 2.9(0-7) |
| | | | | | | P <0.01 | P <0.01 |
| | | | | Pbo | 8.4(7-10) | 7.9(6-9) | 7.6(5-9) |
| | | | | | | P = NS | P = NS |

TABLE 5

| Study | Study | Injection | | | | | | | | |
|---|---|---|---|---|---|---|---|---|---|---|
| Name | Design | site | Duration | VAS for pain | | | | | | |
| Diaz-Llopis IV | R, PC, XO | 40 U to the | 12 months | | Baseline | 1 M | 6 M | 12 M | | |
| et al. 2013 | Study Design | calcaneus and | | | | | | | | |
| Botox vs. | N = 24 | 30 U in the | | | | | | | | |
| steroid | Botox: 70 U | arch of thefoot | | | | | | | | |
| | divided in | Corticosteroid | | | | | | | | |
| | 2 doses | and placebo | | | | | | | | |
| | | the foot | | | | | | | | |
| | | | | Botox | 6.65(1.30) | 3.48(1.47) | 1.78(1.13) | 1.22(1.59) | | |
| | | | | Steroid | N/A | N/A | N/A | N/A | | |
| | | | | | P = 0, 142 between 6 M and 12 M | | | | | |
| | | | | | Corticosteroid group data not reported for VAS. | | | | | |
| Elizondo- | BTX-A (250 U) vs | 200 U to | 6 months | | Baseline | 15 D | 1 M | 2 M | 4 M | 6 M |
| Rodriguez J | Corticosteroids | gastrocnemius | | | | | | | | |
| et al. 2013 | N = 36 | and 50 U to | | | | | | | | |
| Botox vs. | No placebo | the soleus | | | | | | | | |
| Steroid | group | (No BTX-A to | | | | | | | | |
| | | the foot) | | | | | | | | |
| | | Corticosteroids | | | | | | | | |
| | | to the foot | | | | | | | | |
| | | | | BTX-A | 7.1 | 3.0 | 1.9 | 1.6 | 1.5 | 1.1 |
| | | | | Corticosteroids | 7.7 | 4.0 | 3.4 | 3.6 | 3.7 | 3.8 |

Regarding the injection method, injection sites included trigger points described as effective in treating plantar fasciitis. Comparisons thus were made to treatment of plantar fasciitis with dry needling. See, e.g., Eftekharsadat et al, 2016, Med J Islam Repub Iran, 30:401. The study involved a single-blinded randomized clinical trial in 20 patients with chronic heel pain due to plantar fasciitis, where dry needling was administered to subjects in the active arm each week, for four weeks, and the primary outcome involved measuring pain with VAS. The mean VAS score in the dry needling group was significantly lower than the control group after four weeks of intervention (p<0.001); nonetheless, at Week 8, the VAS scores were similar between the dry needling and control groups. See also Table 6 below.

TABLE 6

| | Baseline VAS | | |
|---|---|---|---|
| | Score | Week 4 | Week 8 |
| Dry Needling | 8.2 ± 0.7 | 2.6 ± 1.5 | 3.0 ± 1.2 |
| Control | 7.6 ± 0.9 | 6.6 ± 1.2 | 3.5 ± 1.3 |
| P-value | 0.14 | <0.01 | 0.36 |

Comparisons also were made to treatment of plantar fasciitis by targeting myofascial trigger points. See, e.g., Cochett et al, Phys Ther. 2014 August; 94(8):1083-94; and Moghtaderi, et al, Adv Biomed Res. 2014 Mar. 25; 3:99). One study involved evaluation of dry needling versus a sham treatment of plantar fasciitis, that used parallel groups, blinded participants, and was a randomized control trial. There, the primary outcome measure was first-step pain, measured with VAS (see also Table 7A below). Another study involved extracorporeal shock wave therapy (ESWT) of trigger points to treat plantar fasciitis, that was a randomized, placebo-controlled trial with 40 plantar fasciitis patients, and the primary outcome measure was pain measured with VAS (see also Table 7B below). Targeting of trigger points, whether through dry needling or ESWT resulted in over 50% improvement in VAS score as early as 6 weeks following treatment.

TABLE 7A

| Variable | Real Dry | Sham Dry | Adjusted Mean | |
|---|---|---|---|---|
| First-step | Needling | Needling | Difference | |
| pain (VAS*) | Group | Group | (95% CI) | P |
| Baseline | 67.7 (20.9) | 58.5 (19.5) | | |
| 2 wk | 51.6 (22.0) | 52.7 (23.8) | −8.3 | 0.026* |
| | | | (−15.6 to −1.0) | |
| 4 wk | 38.1 (23.0) | 42.6 (24.1) | −9.2 | 0.058 |
| | | | (−18.7 to 0.3) | |
| 6 wk | 28.6 (19.0) | 38.3 (25.0) | −14.4 | 0.002* |
| | | | (−23.5 to −5.2) | |
| 12 wk | 20.9 (19.4) | 29.9 (23.3) | −12.5 | 0.007 |
| | | | (−21.6 to −1.4) | |

TABLE 7B

| Time | Case Mean +/− SD | Control Mean +/− SD |
|---|---|---|
| Before treatment | 7 +/− 1.3 | 6.6 +/− 1.4 |
| Eight weeks after treatment | 3 +/− 0.9 | 4 +/− 1.1 |
| P-value | <0.001 | 0.02 |

SD: Standard deviation

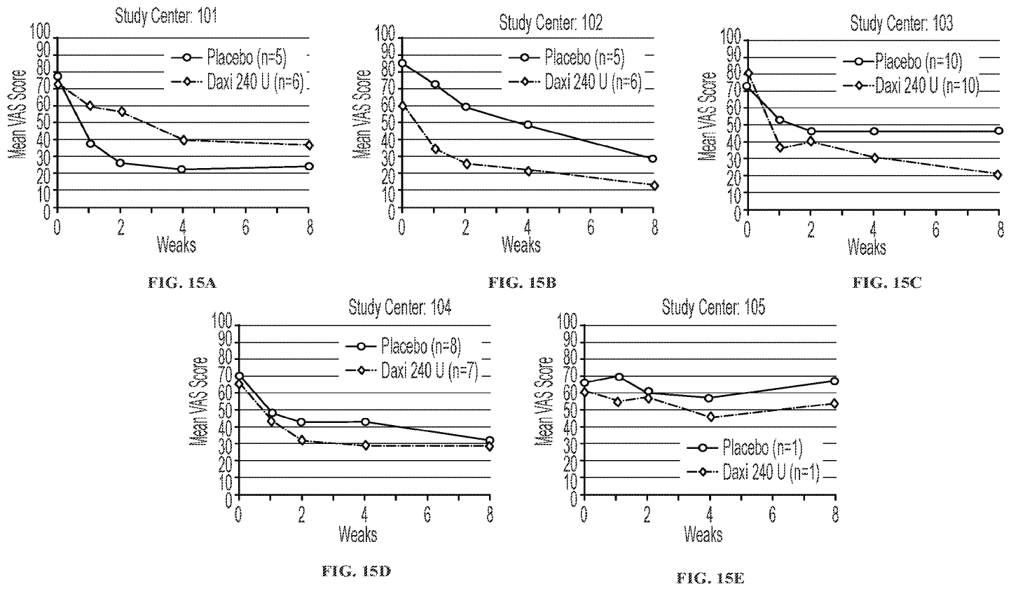

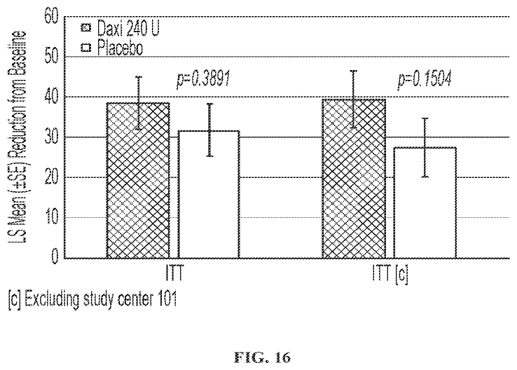

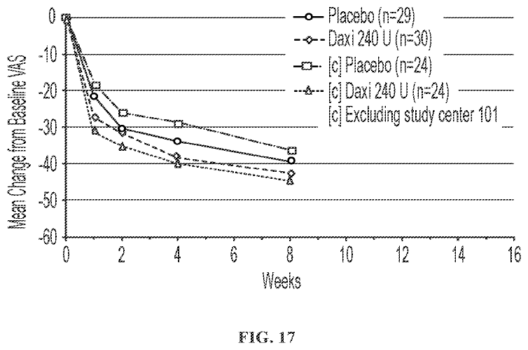

Further sensitivity analyses were conducted on the primary and secondary endpoint results, focusing on individual study centers, as well as considering results with each study center excluded. FIGS. 15A-15E depict VAS pain scores over time by study center, in each of the five study centers of this trial, based on only observed data for subjects in the intent-to-treat (ITT) population. Patients at one study site (site 101) showed larger decreases in the placebo arm on VAS-pain than the test arm, using 240 U Daxi.

FIG. 16 depicts results of a further sensitivity analysis of primary endpoint results for VAS for pain scores at Week 8, excluding results from study center 101; results were based on data from an intent-to-treat (ITT) population analyzed by an ANCOVA model adjusting for study center and baseline VAS scores with the last-observation-carried-forward (LOCF) approach. FIG. 17 depicts results of a sensitivity analysis of secondary endpoint results for change in VAS for pain scores over time, excluding results from study center 101; results were based on only observed data for all subjects in an intent-to-treat (ITT) population.

In summary, reduction in VAS-pain scores at Week 8 observed following treatment with RT002 240 U is consistent with prior botulinum toxin type A studies in plantar fasciitis. However, larger than expected placebo effects were observed in the control group, resulting in non-significant differences between the treatment and control groups in this study.

The primary endpoint at Week 8 was not met as there were similar reductions in VAS for pain scores observed in both RT002 (54%) and placebo (43%) groups (p=0.39). When site 101 is excluded from the analysis, placebo scores decreased on VAS for pain scale (RT002=55.7% and Placebo=37.0%, p=0.15). Similar changes in the secondary (AOFAS and FADI) and exploratory (PFPS) endpoints were observed in both RT002 and placebo groups with no statistical difference observed between the groups.

In the ITT population, larger than expected reduction from baseline in VAS scores were observed in the placebo group. Potential reasons include: site 101 results with poor RT002 response and high placebo response at Week 8; Phase 2 treatment injection sites in the gastrocnemius soleus complex coinciding with known myofascial trigger points for treating pain associated with plantar fasciitis; spontaneous improvement rates of 80-90% that are known to occur in plantar fasciitis populations at 12 months; and/or ongoing supportive measures, such as foot splints.

Safety Data

Table 8 provides a summary of adverse events.

TABLE 8

| | Placebo | Daxi 240 U | All Subjects |
|---|---|---|---|
| | (n = 29) | (n = 30) | (N = 59) |
| Serious AEs (SAE), n (%) | — | — | 1 (1.7%)* |
| Subjects with any AE | 14 (48.3%)^{T} | 18 (60.0%) | 32 (54.2%)^{T} |
| (up to Week 3), n (%) | | | |
| Subjects with any AE, n (%) | 16 (55.2%)^{T} | 18 (60.0%) | 34 (57.6%)^{T} |
| Severe | — | — | 1 (1.7%)* |
| Subjects with any treatment- | 6 (20.7%) | 6 (20.0%) | 12 (20.3%) |
| related⁺ | | | |
| AE, n (%) | | | |
| SAE | 0 | 0 | 0 |
| Subjects with any AE | 0 | 0 | 0 |
| leading to Study | | | |
| Discontinuation, n (%) | | | |

*A severe case of flank pain was also serious, but was considered to be unrelated to study treatment
^{T}Onset of the first AE was after Week 8 for two subjects

Table 9 provides a summary of adverse events that occurred in two or more subjects in the first 8 weeks by the terms used in the study for each of these events (“preferred term”).

TABLE 9

| Preferred | Placebo | Daxi 240 U | All Subjects |
|---|---|---|---|
| Term | (n = 29) | (n = 30) | (N = 59) |
| Any adverse event | 14 (48.3%) | 18 (60.0%) | 32 (54.2%) |
| Injection site | 1 (34%) | 1 (3.3%) | 2 (3.4%) |
| bruising | | | |
| Injection site | 2 (6.9%) | 3 (10.0%) | 5 (8.5%) |
| hemorrhage | | | |
| Injection site | 4 (13.8%) | 4 (13.3%) | 8 (13.6%) |
| pain | | | |
| Arthropod bite | 1 (3.4%) | 1 (3.3%) | 2 (3.4%) |
| Contusion | — | — | 2 (3.4%) |
| Muscle tightness | — | — | 2 (3.4%) |
| Muscular weakness | 1 (3.4%) | 2 (6.7%) | 3 (5.1%) |
| Myalgia | — | — | 2 (3.4%) |
| Pain in extremity | 2(6.9%) | 4 (13.3%) | 6 (10.2%) |
| Hypoaesthesia | — | — | 3 (5.1%) |

Table 10 provides a summary of treatment-related adverse events.

TABLE 10

| Preferred | Placebo | Daxi 240 U | All Subjects |
|---|---|---|---|
| Term | (n = 29) | (n = 30) | (N = 59) |
| Any treatment-related | 6 (20.7%) | 6 (20.0%) | 12 (20.3%) |
| adverse event | | | |
| Injection site bruising | — | — | 1 (1.7%) |
| Injection site erythema | — | — | 1 (1.7%) |
| Injection site Edema | — | — | 1 (1.7%) |
| Injection site pain | 3 (10.3%) | 3 (10.0%) | 6 (10.2%) |
| Edema peripheral | — | — | 1 (1.7%) |
| Muscle tightness | — | — | 2 (3.4%) |
| Muscular weakness | 1 (3.4%) | 1 (3.3%) | 2 (3.4%) |
| Myalgia | — | — | 1 (1.7%) |

RT002 240 U for the treatment of plantar fasciitis appeared to be generally safe and well-tolerated through Week 8 in this Phase 2a Study. The majority of adverse events in both treatment groups were mild in severity. There were no treatment-related serious adverse events. Treatment-related AEs are those possibly, probably, or definitely related to the treatment. The most common treatment-related adverse events for RT002 and placebo were injection site pain (13.3% RT002, 13.8% placebo), pain in extremity (13.3% RT002, 6.9% placebo), injection site hemorrhage (10% RT002, 6.9% placebo) and muscle weakness (3.3% RT002, 3.4% placebo), all of which were classified as mild in severity.

One subject experienced a serious adverse effect (flank pain) which was not considered to be treatment-related. Overall, adverse events occurs in 61% of subjects in the test group and in 55% of subjects in the placebo group, and no subjects in the test group discontinued secondary to adverse events.

Treatment-related adverse events occurred in 21% of subjects in the RT002 group and 20% in the placebo group. The most common treatment-related adverse events were injection site pain (10%) and muscular weakness (3.4%) in both groups.

### Example 2

### Injectable Botulinum Toxin Formulation Showing In Vivo Potency in an Mouse Model

This Example compares in vivo potency of RTT150 and BOTOX. Results indicate that both RTT150 and BOTOX were within 10% of their respective nominal potencies in terms of mouse LD50. Specifically, a 100 U BOTOX vial yielded approximately 109 Units in an in vivo mouse model of potency; and a 160 U RTT150 nominal yielded approximately 170 Units in the same model.

### Example 3

Injectable Botulinum Toxin Formulation in the Treatment of Plantar Fasciitis with Administration to or Near the Plantar Fascia Using Reduced Dose Amounts

This Example describes a clinical study to compare to placebo the safety and efficacy, in managing plantar fasciitis, of a single site injection of an injectable composition of the invention, referred to as RT002, containing botulinum toxin A and a positively charged carrier, as described above. The dose is 80 U or 120 U of RT002.

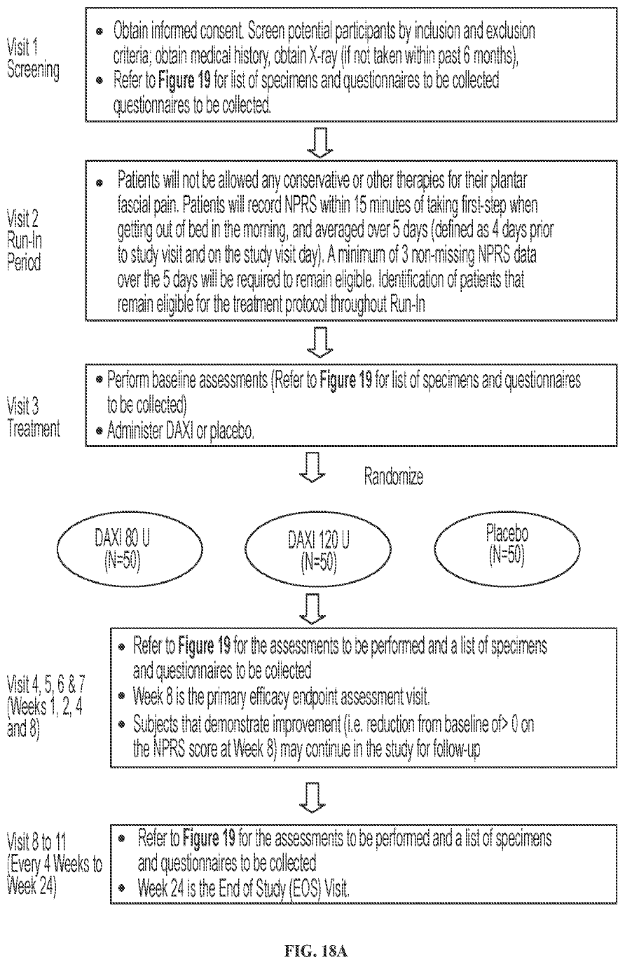

The clinical study is a phase 2, prospective, randomized, double blinded, multicenter, placebo-controlled trial, of one of two doses of DAXI for injection in adult subjects with unilateral plantar fasciitis (PF), lasting up to 24 weeks after injection. The duration for each subject is about 7 months (up to 2 weeks for screening, a 7-day+3 run-in, a single day of treatment, and up to 24 weeks of follow up). Approximately 150 subjects, 18 to 65 years, recruited from up to 20 study centers in the United States (US), are randomized (1:1:1) to receive a plantar fascia injection of RT002 80 U (N=50), RT002 120 U (N=50), or placebo (N=50), respectively. The study combines treatment with use of a standardized written stretching/splinting home therapy program for maximizing toxin effects. See FIG. 18A and FIG. 18B.

Investigators, study site staff (except the study dose preparer), the subject, and sponsor staff including the medical monitor, are blinded to the identity of the subject's assigned treatment. RT002 or placebo is administered in a single injection site into the affected foot. After study drug administration, subjects are followed to assess treatment response, tolerability, and safety up to 24 weeks after the injection.

The primary efficacy endpoint is the change from baseline in a Numeric Pain Rating Scale (NPRS) score (average over 5 days, defined as 4 days prior to study visit and on study visit day) at Week 8. Pain measurement is recorded within first 15 minutes after the first steps out of bed in the morning. In cases of no improvement, Week 8 becomes the Early Termination (ET) Visit for the subject (i.e., the “early” study completer). Subjects who experience a treatment benefit continue to be observed over a 24-week period. The NPRS score, TS, and additional efficacy assessments (e.g., FFI, FAAM, CGIC and PGIC) are performed at pre-specified time points during the study.

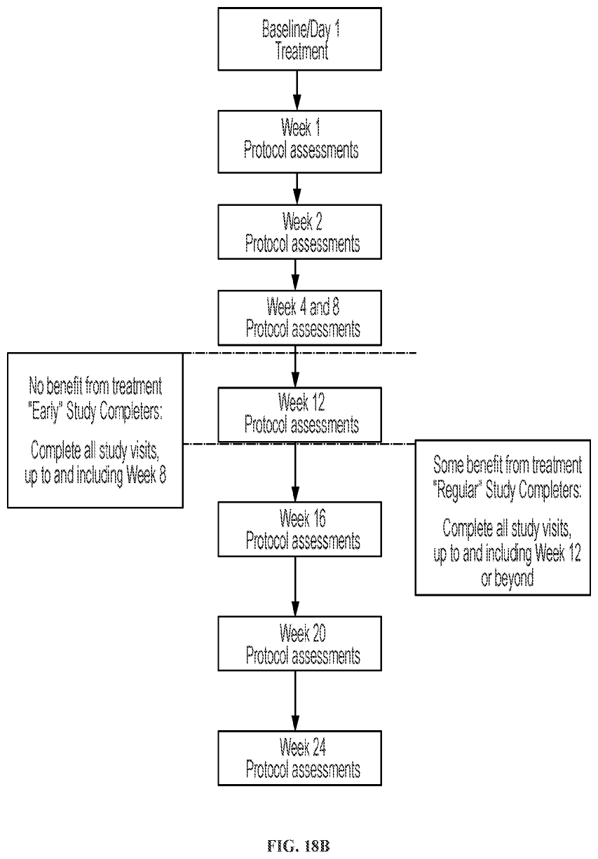

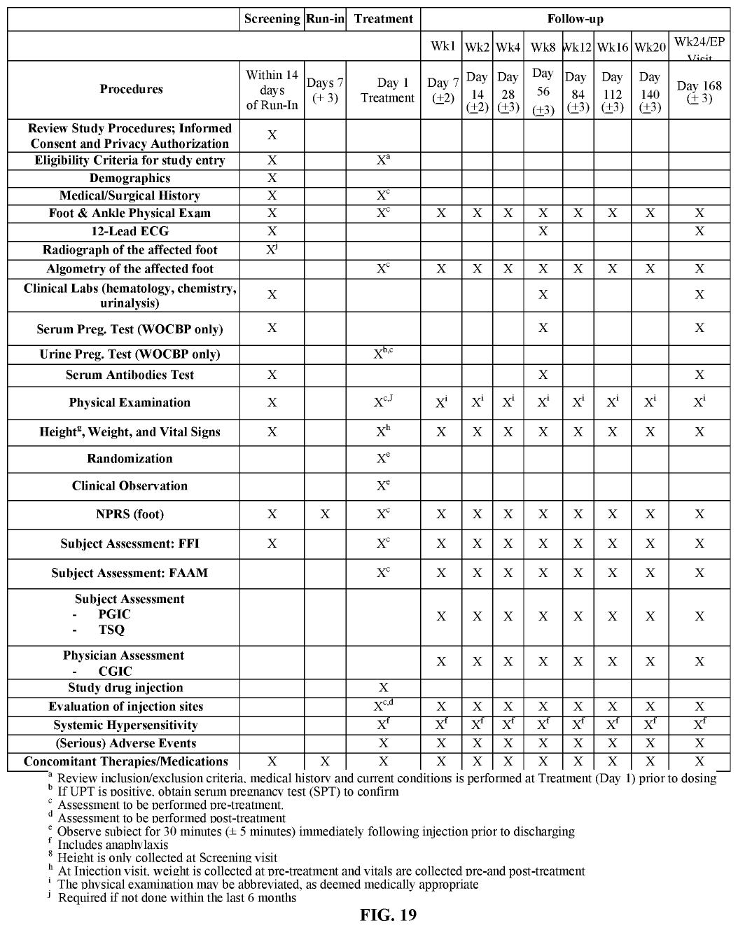

A radiograph is performed at screening to rule out other disease conditions, unless the patient had an X-ray within 6 months prior to study enrollment. Algometry is performed at specified time points to determine the change from baseline in the Pressure Pain Threshold (PPT) over time and to compare these measurements with changes in the NPRS score over time. Safety assessments include laboratory tests (hematology, PT, chemistry, and urinalysis), pregnancy tests for WOCBP; serum antibody tests for BoNTA, physical examinations; vital signs; 12 lead ECGs; injection site evaluations; concomitant medications monitoring; AE monitoring at protocol-specified timepoints; and distant spread of toxin adverse event queries, as outlined in FIG. 19.

The RT002 product is composed of purified 150 kDa botulinum neurotoxin, referred to as RTT150, formulated in a lyophilized powder, as well as containing a positively charged carrier, RTP004. RTT150 is a purified form of the neurotoxin, free of accessory proteins and containing no preservatives, no pooled human serum albumin, bacterial hemagglutins, or other human or animal derived components. This makes RTT150 free of the risk of prion-based and blood-based diseases. RTT150 comprises 160 U RTT150 toxin, per 2 mL vial. Along with RTP004, other components of the formulation include trehalose dihydrate, L-histidine, L-histidine hydrochloride, and polysorbate 20. RTT150 was packaged in 2 mL clear type 1 borosilicated glass single-use vials that are stoppered, over-sealed, and stored at 2-8° C. (not frozen), upright, and protected from light. The product was provided in single-use vials of 100 U of sterile vacuum-dried powder for reconstitution, refrigerated during transit. Placebo to match the 100 U/vial RT002 has the same formulation container closure, and appearance, but without the active ingredient (RT002).

When provided in a 50 U vial, in lyophilized form, the vial contains 0.1 mg polysorbate 20, 36 mg trehalose, and 11.7 μg RTP004 as the carrier, to give a mass ratio of carrier peptide:toxin of 51,000. When provided in a 100 U vial, in lyophilized form, the vial again contains 11.7 μg RTP004 as the carrier, to give a mass ratio of carrier peptide:toxin of 23,400:1 in the 100 U vial. The 11.7 μg RTP004 may also be used with other amounts of toxin as the ratio in mass of carrier to toxin is in the order of 25,000:1.

Study Drug: The investigational product DAXI for Injection (RT002), is a lyophilized product containing purified 150 kDa DAXI formulated in a lyophilized powder containing RTP004. DaxibotulinumtoxinA for injection and placebo are supplied in single-use vials of 100 U/vial of sterile vacuum-dried powder to be reconstituted with sterile, non-preserved 0.9% sodium chloride solution.

Placebo: Placebo is a sterile lyophilized product of inactive ingredients supplied in single-use vials that does not contain toxin to be reconstituted with sterile, non-preserved 0.9% sodium chloride solution.

DaxibotulinumtoxinA for injection and placebo to match are provided in single-use vials, reconstituted with sterile saline for use within 2 hours of preparation. Placebo to match the investigational product is the same in appearance both in the vial before and after reconstitution, and in the syringe. Product is prepared by a trained unblinded dose preparer prior to use. The reconstituted products (active and placebo) are clear, colorless solutions.

Dosing regimen and injection technique: RT002 or placebo are injected on the first study day visit, following the run-in period, and administered to the symptomatic extremity. Ultrasound guidance is used to ensure that the injection is administered to the targeted anatomical area. Subjects are not informed whether they receive RT002 or placebo. A total dose of 1 cc of RT002 80 U or 120 U or placebo is administered as a single site fanned injection using ultrasound guidance into the area of origin of the plantar fascia at the medial calcaneal tuberosity: one third of the content is injected into the fascia and two thirds immediately above (superior to) the plantar fascia in the proximity of the flexor digitorum brevis and the flexor hallucis longus muscles. The ultrasound guidance is designed to assist with correct placement of the needle during injection. Needle size is 25 Gauge in diameter and 1½ inches in length.

Study population: Subjects aged 18-65 with unilateral plantar fasciitis who have failed conservative treatment for ≥3 months and ≤15 months, with an NPRS score of ≥5 and ≤9 who have not previously received botulinum toxin therapy in the lower extremity. Concomitant medications, treatments, and other products not allowed during the trial period are listed in the Prohibited Medications and Treatments table below. Prohibited medication use does not withdraw subject from the trial.

TABLE 11

| | Not Allowed During the |
|---|---|
| Type of Medication or Treatment | Following Time Period |
| Previous injections of botulinum toxins | Any time |
| in the lower extremities or feet | |
| Any conservative treatment other than | Screening through end of trial, |
| the study drug including, but not limited to, | except for physical therapy |
| physical therapy, stretching exercises, | which may be used as a rescue |
| ultrasound, orthotics (over the counter | treatment after the Week 8 |
| or prescribed), taping and | primary endpoint |
| strapping and night splints | |
| Steroid injections in management | Three months prior to screening |
| of plantar fasciitis | through end of trial |
| Extracorporeal therapy of | 30 days prior to treatment through |
| the foot | end of trial |
| Surgery which would affect | Any time |
| ambulation or gait | |
| Antibiotics that may interfere with neuromuscular junction | 3 days prior to treatment and |
| function, for example, aminoglycoside antibiotics (e.g., | 3 days post-treatment |
| gentamicin sulfate, fradiomycin sulfate), polypeptide antibiotics | |
| (e.g., polymyxin B sulfate), tetracycline antibiotics, and | |
| lincomycin antibiotics, except for those contained in topical | |
| antimicrobials | |
| Neuromuscular blocking agents | Screening through end of trial |
| (e.g. curare-like agents) | |
| Any drug which is likely to prolong | Screening through end of trial |
| the QT interval or cause | |
| Torsade de Pointe | |
| Investigational study drugs or devices | 30 days prior to screening |
| | through end of trial |
| Immunosuppressive therapy | 30 days prior to screening |
| | through end of trial |
| Use of concomitant medications, | Two days prior to screening |
| including NSAIDs, analgesics | through end of trial |
| and those which in the Investigator's | |
| opinion, would interfere with | |
| the evaluation of the treatment area | |

Sample size determinations are based on the minimal clinically important difference of 2 points for NPRS (Farrar, 2001; Michener et al., 2011, “Responsiveness of the numeric pain rating scale in patients with shoulder pain and the effect of surgical status,” Journal of sport rehabilitation, 20(1):115). Assuming a standard deviation of 3 points based on the range of NPRS standard deviations on prior PF studies (Cleland et al., 2009, Manual physical therapy and exercise versus electrophysical agents and exercise in the management of plantar heel pain: a multicenter randomized clinical trial, J Orthop Sports Phys Ther, 39(8):573-85; Shashua et al, 2015, “The effect of additional ankle and midfoot mobilizations on plantar fasciitis: a randomized controlled trial,” J Orthop Sports Phys Ther 45(4):265-72), the minimum effect size (Cohen's d) targeted is therefore, 0.7. With a sample size of 126 subjects randomized in a 1:1:1 ratio, the number of subjects per arm is estimated to be 42. The study has at least 85% power to demonstrate a treatment effect difference at a significance level of 0.05 based on a 2-sided two sample t-test when the true effect size is at least 0.7 (i.e., if the true difference between arms in the mean change from baseline in the 0-10 NPRS score on first step at Week 8 is at least 2 points considering a common standard deviation of 3.0 points), and at least 80% power for an effect size of 0.6 considering a conservative standard deviation of 3.2. points. Allowing for 15% attrition rate at Week 8, 50 participants per arm are required, making a total sample size of 150 subjects to be randomized.

Unlike the study in Example 1, subjects here have tried and failed NSAIDs prior to enrollment; and subjects with any degree of obesity are excluded.

Intent-to-Treat (ITT) Population: Efficacy analysis are performed using the intention-to-treat analysis set. This population includes all subjects randomized, who received a study treatment. The ITT population is classified by treatment arm as randomized (i.e., treatment arm based on randomization assignment). All evaluable efficacy data is included in the analysis following the intent-to-treat (ITT) principle. All randomized subjects who received the study treatment (RT002 or placebo) comprise the modified-ITT population and are grouped according to each subject's randomization assignment.

Safety Population: all randomized subjects who received a study treatment.

Randomization: Central randomization is implemented using IWRS/IRT technology and computer-generated randomization. Randomization is stratified by treatment center. The IWRS assigns a unique treatment code, dictating treatment assignment and matching study drug kit for the subject.

Visit Schedule: The run-in period begins no later than 14 days after the screening visit. The run-in period is 7 (+3 days) days to allow for identification of patients that remain eligible throughout the run-in period, prior to randomization. Subjects are treated with investigational product on Treatment Day 1. Post-treatment follow-up visits occur at Weeks 1, 2, 4, 8, 12, 16, 20, and 24/Early Termination. See FIG. 18 and FIG. 19.

Concomitant Therapy: Concomitant medications are any prescription or over-the-counter preparations used by subjects during participation in the trial. No concomitant therapy is allowed during the run-in or study period. Concomitant therapies include but are not limited to RICE (concurrent Rest, Ice, Compression and Elevation), physical therapy, taping, orthotics, night splints, NSAIDs and steroid injections.

Screening Visit: Subjects presenting with heel pain are examined to verify the diagnosis of plantar fasciitis. Then, subjects with plantar fasciitis are screened to determine if they meet the eligibility criteria. The following procedures are completed: complete medical history and physical examination; foot and ankle examination of both feet (including range or motion and motor strength); vital signs (blood pressure [BP], pulse, temperature), weight, and height; blood samples for clinical laboratory (chemistry, hematology, urinalysis), serum antibody tests, and serum pregnancy test (SPT); ECG; foot X-rays if not done within the last six months; concomitant medications/therapies information; NPRS for pain of the foot completed by subject; FFI. Patients are given an ePRO diary (or paper patient diary) to record NPRS daily from now on until Week 24 or ET visit. See FIG. 19.

Run-in Period: NPRS are completed through an ePRO diary (or paper patient diary) entry by the subject, measured within 15 minutes of stepping out the bed in the morning.

Injection Visit: The following procedures are completed pre-treatment: confirming subject eligibility; foot and ankle examination of both feet (including range or motion and motor strength); taking vital signs (blood pressure [BP], pulse, temperature) and weight; urine pregnancy test (UPT); SPT to confirm; concomitant medications/therapies and medical history information; algometry; checking the ePRO patient diary (or paper patient diary) to ensure that the NPRS has been recorded as per protocol requirements (average over 5 days, defined as 4 days prior to study visit and on study visit day); administering subjects' assessments of FFI and FAAM; injection site evaluation to ensure that subject can be injected (i.e., no erythema, edema, itching, etc.).

Post-treatment, the injection site is evaluated and adverse events evaluated.

Follow up Visits: Subjects are evaluated 8 times after injection, at Weeks 1, 2, 4, 8, 12, 16, 20 and 24, or any time subjects terminate prematurely. Acceptable study visit windows can be ±2 days for Weeks 1 and 2; ±3 days for all other visits. The following procedures are completed at each follow-up visit: abbreviated physical examination; foot and ankle examination of both feet (including range or motion and motor strength); vital signs (blood pressure [BP], pulse, temperature) and weight; blood samples for clinical laboratory (chemistry, hematology, urinalysis) at Weeks 8 and 24/Early Termination; algometry of affected foot at specified time points (Weeks 1, 2, 4, 8, 12, 16, 20, and 24/Early Termination); SPT and serum antibody tests Week 8 and 24/Early Termination only; checking the ePRO patient diary (or paper patient diary) to ensure that the NPRS has been recorded as per protocol requirements (average over 5 days, defined as 4 days prior to study visit and on study visit day) (Week 1, 2, 4, 8, 12, 16, 20, and 24/Early Termination); administering subjects' assessments of FFI (Week 1, 2, 4, 8, 12, 16, 20, and 24/Early Termination), GIC (Week 1, 2, 4, 8, 12, 16, 20, and 24/Early Termination), PGIC (Week 1, 2, 4, 8, 12, 16, 20, and 24/Early Termination), TSQ (Week 1, 2, 4, 8, 12, 16, 20, and 24/Early Termination), and FAAM (Week 1, 2, 4, 8 12, 16, 20, and 24/Early Termination); injection site evaluation; concomitant medications/therapies and adverse event(s) information.

Efficacy Assessments: NPRS for Pain; FFI (foot function and pain levels directly can be correlated with treatment outcomes in the Phase 2 study); FAAM; CGIC; PGIC; and TSQ.

Injection Site Evaluation: The injection sites are evaluated at Injection Visit (Day 1) pre- and post-treatment (to determine if there is an immediate reaction to the investigational product), follow-up visits (Weeks 1, 2, 4, 8, 12, 16, 20 and 24 or Early Termination visit), if applicable. The assessment is done as a global evaluation of the injection site, assessing whether or not the following are present: erythema, edema, burning or stinging, itching, bruising, drainage.

Foot and Ankle Examination: Examination for the foot includes ankle, toe, and subtalar range of motion, foot motor strength, location of pain, and examination of the heel fat pad and Tinel's sign. Evaluation is done at Screening, pre-treatment Injection, Weeks 1, 2, 4, 8, 12, 16, 20, and 24//Early Termination visits. The presence of toe deformities, bunions, ulcers, and/or sores is documented. The feet also are examined for signs of swelling, pitting edema, infection, or vascular abnormalities.

Primary Efficacy Endpoint: Change from baseline in the NPRS score (measured as steps taken within first 15 minutes of rising from bed), averaged over 5 days (4 days prior to the study visit and on the study visit day) at Week 8. The primary analysis focuses on reduction from baseline in the NPRS score at 8 weeks, with missing data imputed by the multiple imputation approach. Analysis of covariate (ANCOVA) model including treatment center, treatment group as factors, and baseline pain score as a covariate, is used. In cases of no improvement at Week 8, defined as no change or worsening of NPRS score from baseline, Week 8 becomes the Early Termination (ET) Visit for the subject (i.e., the “early” study completer).

Secondary Efficacy Endpoints: Change from baseline in FFI over time; proportion of subjects with a decrease from baseline of >20% in NPRS over time; time to onset of meaningful pain relief (decrease from baseline ≥20% in NPRS score) following treatment; and median time to loss of >80% treatment benefit achieved at Week 8 on NPRS.

Change from baseline in FFI over time is analyzed using a statistical method that handles repeated measures such as a generalized linear mixed model (GLMM) including treatment center, treatment group, time (visit), and the treatment-visit interaction term as factors. For the proportion of subjects with a decrease from baseline of >20% in NPRS score over time, appropriate statistical models (e.g., Generalized Linear Mixed Model for binary variables) adjusting for relevant covariates is employed to evaluate the treatment effect. For time to event endpoints, Kaplan-Meier survival curves are plotted for each DAXI for injection group for the time-to-event endpoints. Point estimate of median duration and 2-sided, 95% confidence intervals (CIs), are generated using the log-rank test.

Exploratory Efficacy Endpoints: change from baseline in NPRS score over time; proportion of subjects with a decrease from baseline of >10% in NPRS score; proportion of subjects with a decrease from baseline of >30% in NPRS score; change from baseline in Pressure Pain Threshold (PPT) as measured by algometry; proportion of subjects with at least moderate improvement (+2 or higher) on CGIC over time; proportion of subject with improvement (+1 or higher) on the CGIC over time; proportion of subjects with at least moderate improvement (+2 or higher) on PGIC over time; proportion of subject with improvement (+1 or higher) on the PGIC over time; change from baseline in FAAM over time.

Reductions from baseline in the NPRS score over time up also are analyzed using a statistical method that handles repeated measures such as a generalized linear mixed model (GLMM) including treatment center, treatment group, time (visit), and the treatment-visit interaction term as factors, and the baseline NPRS score as covariates. For other exploratory efficacy outcome measures, appropriate statistical models (e.g., ANCOVA or GLM for continuous variables, and chi-squared/Fisher's exact test or logistic regression/Generalized Linear Mixed Model for binary or categorical variables) adjusting for relevant covariates are employed to evaluate the treatment effect. Empirical cumulative distribution function (eCDF) and probability density function (PDF) estimated using kernel density estimation curves are generated to determine a range of clinically meaningful within-patient improvement thresholds using the NPRS score and the P/CGIC as anchor.

Safety Analyses: Safety endpoints are analyzed (e.g., as summary statistics during treatment and/or as change scores from baselines). AEs are coded in accordance with Medical Dictionary for Regulatory Activities (MedDRA)), calculated (e.g., each AE will be counted once only for a given participant), presented (e.g., severity, frequency, time to onset, duration, and relationship of AEs to study intervention presented by System Organ Class (SOC) and preferred term groupings) and information reported about each AE (e.g., start date, stop date, severity, relationship, expectedness, outcome, and duration).

End of Study Definition: A subject completes the study when she/he requests treatment to address pain associated with PF, and it is agreed that another treatment is clinically indicated. Subjects remaining in the study are followed up to Week 24. See FIG. 19.

We claim:

1. A method of administering botulinum toxin to achieve an extended duration therapeutic effect in an individual with plantar fasciitis, the method comprising:
administering by injection a treatment dose of a sterile injectable composition into one or more of the muscles or fascia causing the plantar fasciitis in the individual in need of treatment to achieve the therapeutic effect following treatment with the composition;
wherein the composition comprises a pharmaceutically acceptable diluent suitable for injection; and
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20;
wherein the treatment dose of the botulinum toxin component administered to the individual is about 50 U to about 200 U per injection treatment;
wherein the positively charged carrier is non-covalently associated with the botulinum toxin component; and
wherein the treatment dose of the composition administered by injection to the individual achieves the extended duration therapeutic effect having at least about a six-month duration of effect.

2. A method of treating plantar fasciitis in an individual in need thereof, the method comprising:
administering to the individual by injection to one or more muscles or fascia causing the plantar fasciitis in the individual a composition comprising:
a pharmaceutically acceptable diluent for injection;
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20;
wherein the botulinum toxin component is administered to the individual in a treatment dose amount of about 50 U to about 200 U per injection treatment.
wherein the positively charged carrier is non-covalently associated with the botulinum component; and
wherein the injection of the composition provides a single treatment dose having at least about a six-month duration of effect in reducing the symptoms of plantar fasciitis in the individual, thereby extending treatment interval duration for the individual.

3. A pharmaceutical composition in a sterile injectable formulation for use in administering botulinum toxin to achieve an extended duration therapeutic effect in an individual with plantar fasciitis,
said composition comprising a pharmaceutically acceptable diluent suitable for injection;
a botulinum toxin component in a treatment dose of 50 U to 200 U, wherein said botulinum toxin component is selected from the group consisting of a botulinum toxin complex, a reduced botulinum toxin complex, or a botulinum toxin; and
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20;
wherein the positively charged carrier is non-covalently associated with the botulinum toxin component; and
wherein said treatment dose of the composition achieves the extended duration therapeutic effect having at least about a six-month duration of effect in the individual administered said formulation by injection.

4. A pharmaceutical composition in a sterile injectable formulation for use in reducing the symptoms of plantar fasciitis in an individual in need thereof, said composition comprising:
a botulinum toxin component in a dose of about 50 U to about 200 U, said botulinum toxin component selected from the group consisting of a botulinum toxin complex, a reduced botulinum toxin complex, or a botulinum toxin,
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20; and
a pharmaceutically acceptable diluent for injection;
wherein the positively charged carrier is non-covalently associated with the botulinum toxin component; and
wherein said dose of the composition provides a single treatment having at least about a six-month duration of effect in reducing the symptoms of plantar fasciitis in the individual, thereby extending treatment interval duration for the individual.

5. The method according to claim 1 or claim 2, or the pharmaceutical composition for use according to claim 3 or claim 4, wherein the composition achieves the extended duration effect for at least about 8 months.

6. The method or pharmaceutical composition for use according to claim 5, wherein the composition comprises botulinum toxin of serotype A.

7. The method or pharmaceutical composition for use according to claim 6, wherein the composition comprises botulinum toxin of serotype A having a molecular weight of 150 kDa.

8. The method or pharmaceutical composition for use according to any one of claims 1 to 7, wherein the positively charged polylysine backbone has covalently attached thereto one or more positively charged efficiency groups having the amino acid sequence (gly)ₚ-RGRDDRRQRRR-(gly)_{q} (SEQ ID NO: 1), wherein the subscripts p and q are each independently an integer of from 0 to 20.

9. The method or pharmaceutical composition for use according to any one of claims 1 to 7, wherein the positively charged polylysine backbone has covalently attached thereto one or more positively charged efficiency groups having the amino acid sequence (gly)ₚ-YGRKKRRQRRR-(gly)_{q} (SEQ ID NO: 2), wherein the subscripts p and q are each independently an integer of from 0 to 20.

10. The method or pharmaceutical composition for use according to any one of claims 1 to 7, wherein the positively charged polylysine backbone has covalently attached thereto one or more positively charged efficiency groups having the amino acid sequence (gly)ₚ-RKKRRQRRR-(gly)_{q} (SEQ ID NO: 3), wherein the subscripts p and q are each independently an integer of from 0 to 20.

11. The method or pharmaceutical composition for use according to any one of claims 1 to 10, wherein (i) the subscripts p and q are each independently an integer of from 0 to 8; or (ii) are each independently an integer of from 2 to 5.

12. The method or pharmaceutical composition for use according to any one of claims 1 to 11, wherein the one or more positively charged efficiency groups are attached to both ends of the positively charged polylysine backbone of the positively charged carrier.

13. The method or pharmaceutical composition for use according to claim 12, wherein the positively charged carrier has the amino acid sequence RKKRRQRRRG-(K)₁₅-GRKKRRQRRR (SEQ ID NO: 4).

14. The method or pharmaceutical composition for use according to any one of claims 1 to 13, wherein the composition does not locally diffuse from the site of injection following injection.

15. The method or pharmaceutical composition for use according to any one of claims 1 to 14, wherein the treatment dose of botulinum toxin is administered to the individual in an amount of about 80 U or about 120 per injection treatment.

16. The method or pharmaceutical composition for use according to any one of claims 1 to 15, wherein said positively charged carrier is present in said pharmaceutical composition in an amount of about 0.1 to about 0.3 μg per unit of botulinum toxin component.

17. The method or pharmaceutical composition for use according to claim 16, wherein said positively charged carrier is present in said pharmaceutical composition in an amount of about 0.234 μg per unit of botulinum toxin component.

18. The method or pharmaceutical composition for use according to any one of claims 1 to 17, wherein said excipient comprises at least one component selected from the group consisting of L-Histidine, L-Histidine hydrochloride, polysorbate 20, and trehalose dihydrate.

19. The method or pharmaceutical composition for use according to claim 18, wherein said excipient comprises trehalose dihydrate.

20. The method or pharmaceutical composition for use according to any one of claims 1 to 19, wherein said method or use comprises a single injection of said pharmaceutical composition.

21. The method or pharmaceutical composition for use according to any one of claims 1 to 20, wherein said method or use comprises injection of said pharmaceutical composition into a muscle or fascia of the plantar fascia.

22. The method or pharmaceutical composition for use according to claim 21, wherein the injection occurs in, or proximal to, at least one muscle or fascia selected from the group consisting of plantar fascia, the flexor digitorum brevis, and the flexor hallucis longus.

23. The method or pharmaceutical composition for use according to claim 22, wherein about 80/3 U or 40 U of said botulinum toxin component are injected into said plantar fascia at the medial calcaneal tuberosity; and
about 80×⅔ U or 80 U of said botulinum toxin component are injected immediately superior to the plantar fascia in the proximity of the flexor digitorum brevis and the flexor hallucis longus.

24. The method or pharmaceutical composition for use according to any one of claims 1 to 23, wherein administration of the injection is guided by ultrasound.

25. The method or pharmaceutical composition for use according to any one of claims 1 to 24, wherein the duration of treatment effect comprises at least 6 months through 10 months.

26. A sterile injectable composition comprising:
a botulinum toxin component selected from the group consisting of a botulinum toxin, a botulinum toxin complex, or a reduced botulinum toxin complex, in a dosage amount selected from about 50 U to about 200 U; and
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20; and
a pharmaceutically acceptable diluent for injection;
wherein the positively charged carrier is non-covalently associated with the botulinum toxin component; and
wherein said positively charged carrier is present in said pharmaceutical composition in an amount selected to provide a ratio of about 0.234 μg per unit of botulinum toxin component.

27. The composition according to claim 26, wherein the positively charged carrier has the amino acid sequence RKKRRQRRRG-(K)₁₅-GRKKRRQRRR (SEQ ID NO: 4).

28. The composition according to claim 26 or claim 27, wherein the composition comprises botulinum toxin of serotype A.

29. The composition according to claim 28, wherein the composition comprises botulinum toxin of serotype A having a molecular weight of 150 kDa.

30. The composition according to any one of claims 26 to 29, wherein the treatment dose of the botulinum toxin component administered to the individual is about 80 U or about 120 U.

31. The composition according to any one of claims 28 to 30, wherein the excipient further comprises at least one component selected from L-Histidine, L-Histidine hydrochloride, polysorbate 20, and trehalose dihydrate.

32. The composition according to claim 31, wherein the excipient comprises trehalose dihydrate.

33. A method of treating an individual with plantar fasciitis in need of treatment with injectable botulinum toxin, wherein the method of treatment comprises a treatment course having multiple treatment intervals with prolonged duration of effect and duration time between each treatment interval, the treatment course comprising:
administering by injection an initial treatment dose of a sterile injectable composition into one or more muscles or fascia causing plantar fasciitis in the individual in need of treatment to achieve a therapeutic effect of reducing the symptoms of plantar fasciitis following the initial treatment with the composition;
wherein the composition comprises a pharmaceutically acceptable diluent suitable for injection;
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20;
wherein the botulinum toxin component is administered to the individual in a treatment dose of about 50 U to about 200 U per injection treatment;
wherein the positively charged carrier is non-covalently associated with the botulinum toxin component;
wherein the initial treatment dose of the composition administered by injection to the individual provides a therapeutic duration of effect lasting through at least about six months; and
administering subsequent treatment doses of the composition by injection to the individual at treatment intervals comprising a duration of greater than or equal to about six months to at least about ten months following the initial treatment dose and between each subsequent treatment dose.

34. The method according to claim 33, wherein the composition comprises botulinum toxin of serotype A.

35. The method according to claim 34, wherein the composition comprises botulinum toxin of serotype A having a molecular weight of 150 kDa.

36. The method according to any one of claims 33 to 35, wherein the positively charged polylysine backbone has covalently attached thereto one or more positively charged efficiency groups having the amino acid sequence (gly)ₚ-RGRDDRRQRRR-(gly)_{q} (SEQ ID NO: 1), wherein the subscripts p and q are each independently an integer of from 0 to 20.

37. The method according to any one of claims 33 to 35, wherein the positively charged polylysine backbone has covalently attached thereto one or more positively charged efficiency groups having the amino acid sequence (gly)ₚ-YGRKKRRQRRR-(gly)_{q} (SEQ ID NO: 2), wherein the subscripts p and q are each independently an integer of from 0 to 20.

38. The method according to any one of claims 33 to 35, wherein the positively charged polylysine backbone has covalently attached thereto one or more positively charged efficiency groups having the amino acid sequence (gly)ₚ-RKKRRQRRR-(gly)_{q} (SEQ ID NO: 3), wherein the subscripts p and q are each independently an integer of from 0 to 20.

39. The method according to any one of claims 33 to 38, wherein (i) the subscripts p and q are each independently an integer of from 0 to 8; or (ii) are each independently an integer of from 2 to 5.

40. The method according to any one of claims 33 to 39, wherein the one or more positively charged efficiency groups are attached to both ends of the positively charged polylysine backbone of the positively charged carrier.

41. The method according to any one of claims 33 to 35, wherein the positively charged carrier has the amino acid sequence RKKRRQRRRG-(K)₁₅-GRKKRRQRRR (SEQ ID NO: 4).

42. The method according to any one of claims 33 to 41, wherein the composition does not locally diffuse from the site of injection following injection.

43. The method according to any one of claims 33 to 42, wherein the botulinum toxin is administered to the individual in an amount of about 80 U or about 120 U per injection treatment.

44. The method according to any one of claims 33 to 43, wherein said positively charged carrier is present in said pharmaceutical composition in an amount of about 0.1 to about 0.3 μg per unit of botulinum toxin component.

45. The method according to claim 44, wherein said positively charged carrier is present in said pharmaceutical composition in an amount of about 0.234 μg per unit of botulinum toxin component.

46. The method according to any one of claims 33 to 45, wherein said excipient comprises L-Histidine, L-Histidine hydrochloride, polysorbate 20, and/or trehalose dihydrate.

47. The method according to claim 46, wherein said excipient comprises trehalose dihydrate.

48. The method according to any one of claims 33 to 47, wherein the duration of the treatment interval comprises greater than six months.

49. The method according to any one of claims 33 to 47, wherein the duration of the treatment interval comprises greater than eight months.

50. The method according to any one of claims 33 to 47, wherein the duration of the treatment interval comprises at least six months through ten months.

51. A method of treating plantar fasciitis in an individual in need thereof, the method comprising:
topically administering to the skin overlying to one or more muscles or fascia associated with the plantar fasciitis in the individual, a topical composition comprising:
a pharmaceutically acceptable diluent suitable for topical administration;
an effective amount of a botulinum toxin component selected from the group consisting of a botulinum toxin, a botulinum toxin complex, or a reduced botulinum toxin complex; and
an effective amount of a carrier component comprising a polymeric backbone having covalently attached thereto one or more positively charged efficiency groups,
wherein the carrier component is a positively charged carrier, with the backbone being a positively charged polymeric backbone, or a lipophilic carrier, with the backbone being a hydrophobic oligomeric or polymeric backbone; and
wherein the carrier component is non-covalently associated with the botulinum toxin component,
thereby treating plantar fasciitis.

52. A pharmaceutical composition in a topical formulation for use in treating plantar fasciitis in an individual in need thereof by topical administration to the skin overlying one or more muscles or fascia associated with the plantar fasciitis in said individual, said composition comprising:
a pharmaceutically acceptable diluent suitable for topical administration;
an effective amount of a botulinum toxin component selected from the group consisting of a botulinum toxin complex, a reduced botulinum toxin complex, or a botulinum toxin; and
an effective amount of a carrier component comprising a polymeric backbone having covalently attached thereto one or more positively charged efficiency groups,
wherein the carrier component is a positively charged carrier, with the backbone being a positively charged polymeric backbone, or a lipophilic carrier, with the backbone being a hydrophobic oligomeric or polymeric backbone; and
wherein the carrier component is non-covalently associated with the botulinum toxin component,
thereby treating plantar fasciitis.

53. The method according to claim 51, or the pharmaceutical composition for use according to claim 52, wherein said one or more muscles or fascia is selected from the group consisting of plantar fascia, plantar fascia at the medial calcaneal, flexor digitorum brevis, and flexor hallucis longus.

54. The method or the pharmaceutical composition for use according to claim 53, wherein the carrier component comprises a positively charged carrier, with the backbone being a positively charged polymeric backbone.

55. The method or the pharmaceutical composition for use according to claim 54, wherein the polymeric backbone is a polylysine backbone.

56. The method or the pharmaceutical composition for use according to claim 55, wherein said one or more positively charged efficiency groups has an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20.

57. The method or pharmaceutical composition for use according to claim 56, wherein the positively charged polylysine backbone has covalently attached thereto one or more positively charged efficiency groups having the amino acid sequence (gly)p-RGRDDRRQRRR-(gly)q (SEQ ID NO: 1), wherein the subscripts p and q are each independently an integer of from 0 to 20.

58. The method or pharmaceutical composition for use according to claim 56, wherein the positively charged polylysine backbone has covalently attached thereto one or more positively charged efficiency groups having the amino acid sequence (gly)p-YGRKKRRQRRR-(gly)q (SEQ ID NO: 2), wherein the subscripts p and q are each independently an integer of from 0 to 20.

59. The method or pharmaceutical composition for use according to claim 56, wherein the positively charged polylysine backbone has covalently attached thereto one or more positively charged efficiency groups having the amino acid sequence (gly)p-RKKRRQRRR-(gly)q (SEQ ID NO: 3), wherein the subscripts p and q are each independently an integer of from 0 to 20.

60. The method or pharmaceutical composition for use according to any one of claims 56 to 59, wherein (i) the subscripts p and q are each independently an integer of from 0 to 8; or (ii) are each independently an integer of from 2 to 5.

61. The method or pharmaceutical composition for use according to any one of claims 56 to 60, wherein the one or more positively charged efficiency groups are attached either, or both ends, of the positively charged polylysine backbone of the positively charged carrier.

62. The method or pharmaceutical composition for use according to claim 61, wherein the positively charged carrier has the amino acid sequence RKKRRQRRRG-(K)15-GRKKRRQRRR (SEQ ID NO: 4).

63. The method or the pharmaceutical composition for use according to claim 53, wherein the carrier component comprises a lipophilic carrier, with the backbone being a hydrophobic oligomeric or polymeric backbone.

64. The method or the pharmaceutical composition for use according to claim 63, wherein said one or more efficiency groups is selected from the group consisting of KKRPKPGGGGFFFILVF (SEQ ID NO: 26), FFFILVFGGGKKRPKPG (SEQ ID NO: 27), GGGGKKRPKPG (SEQ ID NO: 28), RKKRRQRRRGGGGFFFILVF (SEQ ID NO: 29), and GGGGRKKRRQRRR (SEQ ID NO: 30).

65. The method or the pharmaceutical composition for use according to claim 64, wherein said lipophilic carrier is selected from the group consisting of palmitoyl-GGRKKRRQRRR (palmitoyl-TAT, SEQ ID NO: 31) and palmitoyl-glyp-KKRPKPG (SEQ ID NO: 11).

66. The method or the pharmaceutical composition for use according to any one of claims 63-65, wherein the composition is contained in a liposome.

67. The method or pharmaceutical composition for use according to any one of claims 51-66, wherein the composition comprises botulinum toxin of serotype A.

68. The method or pharmaceutical composition for use according to claim 67, wherein the composition comprises botulinum toxin of serotype A having a molecular weight of 150 kDa.

69. The method or pharmaceutical composition for use according to any one of claims 51 to 68, wherein the botulinum toxin is contained in a device for dispensing the botulinum toxin, said device being applied topically to the skin of the individual.

70. The method or pharmaceutical composition for use according to claim 69, wherein the device is a skin patch.

71. A method of treating plantar fasciitis in an individual in need thereof, the method comprising:
administering to the individual by a single injection to a muscle or fascia of the plantar fascia of the individual, a composition comprising:
a pharmaceutically acceptable diluent for injection;
a botulinum toxin component that is botulinum toxin of serotype A having a molecular weight of 150 kDa without accessory non-toxin proteins;
a positively charged carrier having the amino acid sequence RKKRRQRRRG-(K)₁₅-GRKKRRQRRR (SEQ ID NO: 4);
wherein the botulinum toxin component is administered to the individual in a treatment dose amount of about 80 U or about 120 U per injection treatment;
wherein the positively charged carrier is non-covalently associated with the botulinum component; and
wherein the injection of the composition provides a single treatment dose having at least about a 26-week duration of effect in reducing pain associated with the plantar fasciitis of the individual,
thereby extending treatment interval duration for the individual.

72. A pharmaceutical composition in a sterile injectable formulation for use in treating plantar fasciitis in an individual in need thereof, said composition comprising:
a botulinum toxin component in a dose of about 80 U or about 120 U, said botulinum toxin component consisting of serotype A having a molecular weight of 150 kDa without accessory non-toxin proteins,
a positively charged carrier having the amino acid sequence RKKRRQRRRG-(K)₁₅-GRKKRRQRRR (SEQ ID NO: 4); and
a pharmaceutically acceptable diluent for injection;
wherein the positively charged carrier is non-covalently associated with the botulinum toxin component; and
wherein said dose of the composition provides a single injection treatment to a muscle or fascia of the plantar fascia of the individual to give at least about a 26-week duration of effect in reducing pain associated with the plantar fasciitis of the individual, thereby extending treatment interval duration for the individual.

73. The method according to claim 71, or the pharmaceutical composition for use according to claim 72, wherein the composition achieves an extended duration of effect for at least about 27 weeks.

74. The method according to claim 71, or the pharmaceutical composition for use according to claim 72, wherein the composition achieves an extended duration of effect for at least about 28 weeks.

75. The method according to claim 71, or the pharmaceutical composition for use according to claim 72, wherein the composition achieves an extended duration of effect for at least about 30 weeks.

76. The method or pharmaceutical composition for use according to any one of claims 71 to 75, wherein said positively charged carrier is present in said pharmaceutical composition in an amount of about 0.1 to about 0.3 μg per unit of botulinum toxin component.

77. The method or pharmaceutical composition for use according to claim 76, wherein said positively charged carrier is present in said pharmaceutical composition in an amount of about 0.234 μg per unit of botulinum toxin component.

78. The method or pharmaceutical composition for use according to any one of claims 71 to 77, wherein said excipient comprises at least one component selected from the group consisting of L-Histidine, L-Histidine hydrochloride, polysorbate 20, and trehalose dihydrate.

79. The method or pharmaceutical composition for use according to claim 78, wherein said excipient comprises trehalose dihydrate.

80. The method or pharmaceutical composition for use according to any one of claims 71-79, wherein the reduction in the at least one symptom of plantar fasciitis comprises a reduction in the severity of pain.

81. The method or pharmaceutical composition for use according to any one of claim 71-80, wherein the administration comprises a single injection to one or more muscle and fascia selected from the group consisting of plantar fascia, flexor digitorum brevis, and flexor hallucis longus.

82. The method or pharmaceutical composition for use according to claim 81, wherein about 80/3 U or about 40 U of said botulinum toxin component are injected into said plantar fascia at the medial calcaneal tuberosity; and
about 80×⅔ U or 80 U of said botulinum toxin component are injected immediately superior to the plantar fascia in the proximity of the flexor digitorum brevis and the flexor hallucis longus.

83. The method or pharmaceutical composition for use according to any one of claim 71-82, wherein administration of the injection is guided by ultrasound.

84. The method or pharmaceutical composition for use according to any one of claims 71 to 82, wherein the reduction in pain endures for at least about 4 weeks in over 55% of individuals each administered the pharmaceutical composition.

85. The method or pharmaceutical composition for use according to claim 84, wherein the reduction in pain endures for at least about 4 weeks in over 70% of individuals each administered the pharmaceutical composition.

86. The method or pharmaceutical composition for use according to any one of claims 71 to 82, wherein the reduction in pain endures for at least about 16 weeks in over 35% of individuals each administered the pharmaceutical composition.

87. The method or pharmaceutical composition for use according to claim 86, wherein the reduction in pain endures for at least about 16 weeks in over 50% of individuals each administered the pharmaceutical composition.

88. The method or pharmaceutical composition for use according to any one of claims 71 to 82, wherein the reduction in pain endures for at least about 24 weeks in over 15% of individuals each administered the pharmaceutical composition.

89. The method or pharmaceutical composition for use according to claim 88, wherein the reduction in the pain endures for at least about 24 weeks in over 25% of individuals each administered the pharmaceutical composition.

90. The method or pharmaceutical composition for use according to any one of claims 71-89, wherein the reduction comprises a reduction in the severity of pain associated with the plantar fasciitis as measured by at least one assessment method selected from the group consisting of Numeric Pain Rating Scale (NPRS), Foot Function Index (FFI), Patient Global Impression of Change (PGIC), Clinician Global Impression of Change (CGIC), and Treatment Satisfaction Questionnaire (TSQ).

91. A method of treating plantar fasciitis in an individual in need thereof, the method comprising:
administering to the individual by injection to one or more muscles or fascia associated with the plantar fasciitis of the individual a composition comprising:
a pharmaceutically acceptable diluent for injection;
a botulinum toxin component that is botulinum toxin of serotype A having a molecular weight of 150 kDa without accessory non-toxin proteins;
a positively charged carrier having the amino acid sequence RKKRRQRRRG-(K)₁₅-GRKKRRQRRR (SEQ ID NO: 4);
wherein the botulinum toxin component is administered to the individual in a treatment dose amount of about 240 U per injection treatment;
wherein the positively charged carrier is non-covalently associated with the botulinum component; and
wherein the injection of the composition provides a single treatment dose that reduces pain associated with plantar fasciitis by at least 50% 8 weeks following treatment.

92. A pharmaceutical composition in a sterile injectable formulation for use in treating plantar fasciitis in an individual in need thereof, said composition comprising:
a botulinum toxin component in a dose of about 240 U, said botulinum toxin component consisting of serotype A having a molecular weight of 150 kDa without accessory non-toxin proteins,
a positively charged carrier having the amino acid sequence RKKRRQRRRG-(K)₁₅-GRKKRRQRRR (SEQ ID NO: 4); and
a pharmaceutically acceptable diluent for injection;
wherein the positively charged carrier is non-covalently associated with the botulinum toxin component; and
wherein said dose of the composition provides a single treatment that reduces pain associated with plantar fasciitis by at least 50% 8 weeks following treatment.

93. The method or pharmaceutical composition for use according to claim 91 or 92, wherein said positively charged carrier is present in said pharmaceutical composition in an amount of about 0.1 to about 0.3 μg per unit of botulinum toxin component.

94. The method or pharmaceutical composition for use according to claim 93, wherein said positively charged carrier is present in said pharmaceutical composition in an amount of about 0.234 μg per unit of botulinum toxin component.

95. The method or pharmaceutical composition for use according to any one of claims 91 to 94, wherein said excipient comprises at least one component selected from the group consisting of L-Histidine, L-Histidine hydrochloride, polysorbate 20, and trehalose dihydrate.

96. The method or pharmaceutical composition for use according to claim 95, wherein said excipient comprises trehalose dihydrate.

97. The method or pharmaceutical composition for use according to any one of claim 91-96, wherein the administration comprises at least one injection into one or more muscle and fascia selected from the group consisting of plantar fascia, gastrocnemius-soleus complex, periosteum, quadratus plantae, and a short flexor.

98. The method or pharmaceutical composition for use according to claim 97, wherein about 160 U of said botulinum toxin component are injected into the gastrocnemius-soleus complex and about 80 U of said botulinum toxin component are injected into the plantar fascia, periosteum, quadratus plantae, and a short flexor.

99. The method or pharmaceutical composition for use according to any one of claims 91-98, wherein the reduction comprises a reduction in the severity of pain associated with the plantar fasciitis as measured by visual analog score (VAS) for pain of by Numeric Pain Rating Scale (NPRS).

100. The method or pharmaceutical composition for use according to any one of claims 91-99, wherein the pharmaceutical composition comprises 0.1 mg polysorbate 20 and 36 mg trehalose dehydrate per 50 U of toxin.
